SEQUENCE LISTING (126 sequences)

SEQ ID NO: 1 - protein, 10 aa, Homo sapiens
GAILGRSETQ

SEQ ID NO: 2 - protein, 8 aa, Homo sapiens
GRGEAETR

SEQ ID NO: 3 - protein, 5 aa, Homo sapiens
ECLFF

SEQ ID NO: 4 - protein, 5 aa, Homo sapiens
ECIYY

SEQ ID NO: 5 - protein, 10 aa, Homo sapiens
NANWEKDRTN

SEQ ID NO: 6 - protein, 10 aa, Homo sapiens
NANWELERTN

SEQ ID NO: 7 - protein, 7 aa, Homo sapiens
QTGVEPC

SEQ ID NO: 8 - protein, 7 aa, Homo sapiens
QSGLERC

SEQ ID NO: 9 - protein, 7 aa, Homo sapiens
YGDKDKR

SEQ ID NO: 10 - protein, 7 aa, Homo sapiens
EGEQDKR

SEQ ID NO: 11 - protein, 10 aa, Homo sapiens
RHCFATWKNI

SEQ ID NO: 12 - protein, 7 aa, Homo sapiens
HCFATWK

SEQ ID NO: 13 - protein, 10 aa, Homo sapiens
LHCYASWRNS

SEQ ID NO: 14 - protein, 7 aa, Homo sapiens
HCYASWR

SEQ ID NO: 15 - protein, 10 aa, Homo sapiens
SIEIVKQGCW

SEQ ID NO: 16 - protein, 8 aa, Homo sapiens
EIVKQGCW

SEQ ID NO: 17 - protein, 10 aa, Homo sapiens
TIELVKKGCW

SEQ ID NO: 18 - protein, 8 aa, Homo sapiens
ELVKKGCW

SEQ ID NO: 19 - protein, 12 aa, Homo sapiens
LDDINCYDRTDC

SEQ ID NO: 20 - protein, 12 aa, Homo sapiens
LDDFNCYDRQEC

SEQ ID NO: 21 - protein, 6 aa, Homo sapiens
KDSPEV

SEQ ID NO: 22 - protein, 6 aa, Homo sapiens
EENPQV

SEQ ID NO: 23 - protein, 6 aa, Homo sapiens
YFCCCE

SEQ ID NO: 24 - protein, 6 aa, Homo sapiens
GNMCNE

SEQ ID NO: 25 - protein, 6 aa, Homo sapiens
GNFCNE

SEQ ID NO: 26 - protein, 5 aa, Homo sapiens
KFSYF

SEQ ID NO: 27 - protein, 5 aa, Homo sapiens
RFTHL

SEQ ID NO: 28 - protein, 10 aa, Homo sapiens
PEMEVTQPTS

SEQ ID NO: 29 - protein, 18 aa, Homo sapiens
PEAGGPEVTYEPPPTAPT

SEQ ID NO: 30 - protein, 109 aa, Homo sapiens
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 31 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 33 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 34 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 35 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 4 aa, synthetic construct
GGGA

SEQ ID NO: 37 - protein, 4 aa, synthetic construct
GGGS

SEQ ID NO: 38 - protein, 4 aa, synthetic construct
GGGG

SEQ ID NO: 39 - protein, 5 aa, synthetic construct
GGGGA

SEQ ID NO: 40 - protein, 5 aa, synthetic construct
GGGGS

SEQ ID NO: 41 - protein, 5 aa, synthetic construct
GGGGG

SEQ ID NO: 42 - protein, 4 aa, synthetic construct
GGAG

SEQ ID NO: 43 - protein, 4 aa, synthetic construct
GGSG

SEQ ID NO: 44 - protein, 4 aa, synthetic construct
AGGG

SEQ ID NO: 45 - protein, 4 aa, synthetic construct
SGGG

SEQ ID NO: 46 - protein, 4 aa, synthetic construct
GAGA

SEQ ID NO: 47 - protein, 4 aa, synthetic construct
GSGS

SEQ ID NO: 48 - protein, 6 aa, synthetic construct
GAGAGA

SEQ ID NO: 49 - protein, 6 aa, synthetic construct
GSGSGS

SEQ ID NO: 50 - protein, 8 aa, synthetic construct
GAGAGAGA

SEQ ID NO: 51 - protein, 8 aa, synthetic construct
GSGSGSGS

SEQ ID NO: 52 - protein, 10 aa, synthetic construct
GAGAGAGAGA

SEQ ID NO: 53 - protein, 10 aa, synthetic construct
GSGSGSGSGS

SEQ ID NO: 54 - protein, 12 aa, synthetic construct
GAGAGAGAGAGA

SEQ ID NO: 55 - protein, 12 aa, synthetic construct
GSGSGSGSGSGS

SEQ ID NO: 56 - protein, 6 aa, synthetic construct
GGAGGA

SEQ ID NO: 57 - protein, 6 aa, synthetic construct
GGSGGS

SEQ ID NO: 58 - protein, 9 aa, synthetic construct
GGAGGAGGA

SEQ ID NO: 59 - protein, 9 aa, synthetic construct
GGSGGSGGS

SEQ ID NO: 60 - protein, 12 aa, synthetic construct
GGAGGAGGAGGA

SEQ ID NO: 61 - protein, 12 aa, synthetic construct
GGSGGSGGSGGS

SEQ ID NO: 62 - protein, 8 aa, synthetic construct
GGAGGGAG

SEQ ID NO: 63 - protein, 8 aa, synthetic construct
GGSGGGSG

SEQ ID NO: 64 - protein, 12 aa, synthetic construct
GGAGGGAGGGAG

SEQ ID NO: 65 - protein, 12 aa, synthetic construct
GGSGGGSGGGSG

SEQ ID NO: 66 - protein, 15 aa, synthetic construct
GGGGAGGGGAGGGGA

SEQ ID NO: 67 - protein, 15 aa, synthetic construct
GGGGSGGGGSGGGGS

SEQ ID NO: 68 - protein, 5 aa, synthetic construct
GGGAG

SEQ ID NO: 69 - protein, 6 aa, synthetic construct
GGGAGG

SEQ ID NO: 70 - protein, 7 aa, synthetic construct
GGGAGGG

SEQ ID NO: 71 - protein, 4 aa, synthetic construct
AAAL

SEQ ID NO: 72 - protein, 4 aa, synthetic construct
AAAK

SEQ ID NO: 73 - protein, 4 aa, synthetic construct
AAAR

SEQ ID NO: 74 - protein, 14 aa, synthetic construct
EGKSSGSGSESKST

SEQ ID NO: 75 - protein, 12 aa, synthetic construct
GSAGSAAGSGEF

SEQ ID NO: 76 - protein, 12 aa, synthetic construct
AEAAAKEAAAKA

SEQ ID NO: 77 - protein, 18 aa, synthetic construct
KESGSVSSEQLAQFRSLD

SEQ ID NO: 78 - protein, 10 aa, synthetic construct
GENLYFQSGG

SEQ ID NO: 79 - protein, 8 aa, synthetic construct
SACYCELS

SEQ ID NO: 80 - protein, 5 aa, synthetic construct
RSIAT

SEQ ID NO: 81 - protein, 17 aa, synthetic construct
RPACKIPNDLKQKVMNH

SEQ ID NO: 82 - protein, 36 aa, synthetic construct
GGSAGGSGSGSSGGSSGASGTGTAGGTGSGSGTGSG

SEQ ID NO: 83 - protein, 17 aa, synthetic construct
AAANSSIDLISVPVDSR

SEQ ID NO: 84 - protein, 36 aa, synthetic construct
GGSGGGSEGGGSEGGGSEGGGSEGGGSEGGGSGGGS

SEQ ID NO: 85 - protein, 5 aa, synthetic construct
EAAAK

SEQ ID NO: 86 - protein, 5 aa, synthetic construct
PAPAP

SEQ ID NO: 87 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 11 aa, synthetic construct
DICLPRWGCLW

SEQ ID NO: 89 - protein, 93 aa, Homo sapiens
GPVEVFITETPSQPNSHPIQWNAPQPSHISKYILRWRPKNSVGRWKEATIPGHLNSYTIKGLKPGVVYEGQLISIQQYGHQEVTRFDFTTTST

SEQ ID NO: 90 - protein, 609 aa, Homo sapiens [sequence omitted]

SEQ ID NO: 91 - protein, 6 aa, Homo sapiens
NPVTPK

SEQ ID NO: 92 - protein, 10 aa, synthetic construct
  VARIANT 1: note = Any amino acid
  VARIANT 3: note = Any amino acid
  VARIANT 5: note = Any amino acid
  VARIANT 7: note = Any amino acid
  VARIANT 9: note = Any amino acid
  VARIANT 1..10: note = This sequence may encompass 1-5 XP repeating units
XPXPXPXPXP

SEQ ID NO: 93-95 - skipped (3 entries)

SEQ ID NO: 96 - protein, 109 aa, synthetic construct
GAILGRSETQECLFFNANWEKDRTNQSGLERCYGDKDKRRHCFATWKNISGSIEIVKQGCWLDDINCYDRTDCVEKKDSPEVYFCCCEGNMCNEKFSYFPEMEVTQPTS

SEQ ID NO: 97 - protein, 109 aa, synthetic construct
GAILGRSETQECLFFNANWEKDRTNQTGVEPCYGDKDKRLHCYASWRNSSGSIEIVKQGCWLDDINCYDRTDCVEKKDSPEVYFCCCEGNMCNEKFSYFPEMEVTQPTS

SEQ ID NO: 98 - protein, 109 aa, synthetic construct
GAILGRSETQECLFFNANWEKDRTNQTGVEPCYGDKDKRRHCFATWKNISGTIELVKKGCWLDDINCYDRTDCVEKKDSPEVYFCCCEGNMCNEKFSYFPEMEVTQPTS

SEQ ID NO: 99 - protein, 109 aa, synthetic construct
GAILGRSETQECLFFNANWEKDRTNQTGVEPCYGDKDKRRHCFATWKNISGSIEIVKQGCWLDDINCYDRTDCVATKDSPEVYFCCCEGNMCNEKFSYFPEMEVTQPTS

SEQ ID NO: 100 - protein, 109 aa, synthetic construct
GAILGRSETQECLFFNANWEKDRTNQTGVEPCYGDKDKRRHCFATWKNISGSIEIVKQGCWLDDINCYDRTDCVEKKDSPEVYFCCCEGNMCNERFTHLPEMEVTQPTS

SEQ ID NO: 101 - protein, 109 aa, synthetic construct
GAILGRSETQECIYYNANWEKDRTNQTGVEPCYGDKDKRRHCFATWKNISGSIEIVKQGCWLDDINCYDRTDCVEKKDSPEVYFCCCEGNMCNERFTHLPEMEVTQPTS

SEQ ID NO: 102 - protein, 109 aa, synthetic construct
GAILGRSETQECIYYNANWEKDRTNQTGVEPCYGDKDKRRHCFATWKNISGTIELVKKGCWLDDINCYDRTDCVEKKDSPEVYFCCCEGNMCNEKFSYFPEMEVTQPTS

SEQ ID NO: 103 - protein, 109 aa, synthetic construct
GAILGRSETQECLEFNANWEKDRTNQSGLERCYGDKDKRRHCFATWKNISGTIELVKKGCWLDDINCYDRTDCVEKKDSPEVYFCCCEGNMCNEKFSYFPEMEVTQPTS

SEQ ID NO: 104 - protein, 107 aa, synthetic construct
GRGEAETRECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 105 - protein, 109 aa, synthetic construct
GAILGRSETQECLFFNANWELERTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 106 - protein, 109 aa, synthetic construct
GAILGRSETQECLFFNANWEKDRTNQTGVEPCEGEQDKRRHCFATWKNISGSIEIVKQGCWLDDINCYDRTDCVEKKDSPEVYFCCCEGNMCNEKFSYFPEMEVTQPTS

SEQ ID NO: 107 - protein, 109 aa, synthetic construct
GAILGRSETQECLFFNANWEKDRTNQTGVEPCYGDKDKRRHCFATWKNISGSIEIVKQGCWLDDFNCYDRQECVEKKDSPEVYFCCCEGNMCNEKFSYFPEMEVTQPTS

SEQ ID NO: 108 - protein, 109 aa, synthetic construct
GAILGRSETQECLFFNANWEKDRTNQTGVEPCYGDKDKRRHCFATWKNISGSIEIVKQGCWLDDINCYDRTDCVEKEENPQVYFCCCEGNMCNEKFSYFPEMEVTQPTS

SEQ ID NO: 109 - protein, 109 aa, synthetic construct
GAILGRSETQECLFFNANWEKDRTNQTGVEPCYGDKDKRRHCFATWKNISGSIEIVKQGCWLDDINCYDRTDCVEKKDSPEVYFCCCEGNFCNEKFSYFPEMEVTQPTS

SEQ ID NO: 110 - protein, 117 aa, synthetic construct
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEAGGPEVTYEPPPTAPT

SEQ ID NO: 111 - protein, 115 aa, synthetic construct
GRGEAETRECIYYNANWELERTNQTGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 112 - protein, 115 aa, synthetic construct
GRGEAETRECIYYNANWELERTNQSGLERCEGEQDKRRHCFATWKNISGTIELVKKGCWLDDFNCYDRQECVATEENPQVYFCCCEGNFCNERFTHLPEAGGPEVTYEPPPTAPT

SEQ ID NO: 113 - protein, 115 aa, synthetic construct
GRGEAETRECIYYNANWELERTNQSGLERCEGEQDKRLHCYASWRNSSGSIEIVKQGCWLDDFNCYDRQECVATEENPQVYFCCCEGNFCNERFTHLPEAGGPEVTYEPPPTAPT

SEQ ID NO: 114 - protein, 115 aa, synthetic construct
GRGEAETRECIYYNANWELERTNQSGLERCEGEQDKRLHCYASWRNSSGTIELVKKGCWLDDFNCYDRQECVEKEENPQVYFCCCEGNFCNERFTHLPEAGGPEVTYEPPPTAPT

SEQ ID NO: 115 - protein, 115 aa, synthetic construct
GRGEAETRECIYYNANWELERTNQSGLERCEGEQDKRLHCYASWRNSSGTIELVKKGCWLDDFNCYDRQECVATEENPQVYFCCCEGNFCNEKFSYFPEAGGPEVTYEPPPTAPT

SEQ ID NO: 116 - protein, 115 aa, synthetic construct
GRGEAETRECLFFNANWELERTNQSGLERCEGEQDKRLHCYASWRNSSGTIELVKKGCWLDDFNCYDRQECVATEENPQVYFCCCEGNFCNEKFSYFPEAGGPEVTYEPPPTAPT

SEQ ID NO: 117 - protein, 115 aa, synthetic construct
GRGEAETRECLFFNANWELERTNQSGLERCEGEQDKRLHCYASWRNSSGSIEIVKQGCWLDDFNCYDRQECVATEENPQVYFCCCEGNFCNERFTHLPEAGGPEVTYEPPPTAPT

SEQ ID NO: 118 - protein, 115 aa, synthetic construct
GRGEAETRECIYYNANWELERTNQTGVEPCEGEQDKRLHCYASWRNSSGSIEIVKQGCWLDDFNCYDRQECVATEENPQVYFCCCEGNFCNERFTHLPEAGGPEVTYEPPPTAPT

SEQ ID NO: 119 - protein, 115 aa, synthetic construct
GRGEAETRECLFFNANWELERTNQTGVEPCEGEQDKRRHCFATWKNISGSIEIVKQGCWLDDFNCYDRQECVATEENPQVYFCCCEGNFCNERFTHLPEAGGPEVTYEPPPTAPT

SEQ ID NO: 120 - protein, 117 aa, synthetic construct
GAILGRSETQECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 121 - protein, 115 aa, synthetic construct
GRGEAETRECIYYNANWEKDRTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 122 - protein, 115 aa, synthetic construct
GRGEAETRECIYYNANWELERTNQSGLERCYGDKDKRLHCYASWRNSSGTIELVKKGCWLDDFNCYDRQECVATEENPQVYFCCCEGNFCNERFTHLPEAGGPEVTYEPPPTAPT

SEQ ID NO: 123 - protein, 115 aa, synthetic construct
GRGEAETRECIYYNANWELERTNQSGLERCEGEQDKRLHCYASWRNSSGTIELVKKGCWLDDINCYDRTDCVATEENPQVYFCCCEGNFCNERFTHLPEAGGPEVTYEPPPTAPT

SEQ ID NO: 124 - protein, 115 aa, synthetic construct
GRGEAETRECIYYNANWELERTNQSGLERCEGEQDKRLHCYASWRNSSGTIELVKKGCWLDDFNCYDRQECVATKDSPEVYFCCCEGNFCNERFTHLPEAGGPEVTYEPPPTAPT

SEQ ID NO: 125 - protein, 115 aa, synthetic construct
GRGEAETRECIYYNANWELERTNQSGLERCEGEQDKRLHCYASWRNSSGTIELVKKGCWLDDFNCYDRQECVATEENPQVYFCCCEGNMCNERFTHLPEAGGPEVTYEPPPTAPT

SEQ ID NO: 126 - protein, 107 aa, synthetic construct
GRGEAETRECIYYNANWELERTNQSGLERCEGEQDKRLHCYASWRNSSGTIELVKKGCWLDDFNCYDRQECVATEENPQVYFCCCEGNFCNERFTHLPEMEVTQPTS

(19) United States
(12) Patent Application Publication
(10) Pub. No.: US 2025/0270287 A1
(43) Pub. Date: Aug. 28, 2025

(54) ACTIVIN RECEPTOR TYPE II CHIMERAS AND METHODS OF USE THEREOF

(71) Applicant: Keros Therapeutics, Inc., Lexington, MA (US)

(72) Inventors: Jasbir S. SEEHRA, Lexington, MA (US); Jennifer LACHEY, Lincoln, MA (US); Claire TSENG, Newton, MA (US)

(21) Appl. No.: 19/204,105

(22) Filed: May 9, 2025

Related U.S. Application Data
(63) Continuation of application No. PCT/US2023/079222, filed on Nov. 9, 2023.
(60) Provisional application No. 63/424,385, filed on Nov. 10, 2022.

(51) Int. Cl. C07K 14/71; A61K 38/00; A61P 3/04; A61P 7/00; A61P 7/04; A61P 7/06; A61P 11/00; A61P 13/12; A61P 19/08; A61P 19/10; A61P 21/00

(52) U.S. Cl. CPC C07K 14/71; A61P 3/04; A61P 7/00; A61P 7/04; A61P 7/06; A61P 11/00; A61P 13/12; A61P 19/08; A61P 19/10; A61P 21/00; A61K 38/00; C07K 2319/30; C07K 2319/31

(57) ABSTRACT

The invention features polypeptides that include an extracellular ActRII chimera. In some embodiments, a polypeptide of the invention includes an extracellular ActRII chimera fused to an Fc domain or moiety. The invention also features pharmaceutical compositions and methods of using the polypeptides to treat diseases and conditions involving weakness or atrophy of muscles, bone damage, low red blood cell levels (e.g., anemia or blood loss), low platelet levels (e.g., thrombocytopenia), low neutrophil levels (e.g., neutropenia), fibrosis, metabolic disorders, pulmonary hypertension, and/or diseases and conditions that can be treated with erythropoietin or an erythropoiesis-stimulating agent.

### SEQUENCE LISTING

The instant application contains a Sequence Listing which has been submitted electronically in XML file format and is hereby incorporated by reference in its entirety. Said XML copy, created on May 5, 2025, is named “51184-049003_Sequence_Listing_5_5_25” and is 115,698 bytes in size.

### BACKGROUND OF THE INVENTION

Duchenne muscular dystrophy (DMD), facioscapulohumeral muscular dystrophy (FSHD), inclusion body myositis (IBM), and amyotrophic lateral sclerosis (ALS) are examples of muscle diseases that involve weakness and atrophy of muscles and/or motor neurons that control voluntary muscle movements. DMD is caused by mutations in the X-linked dystrophin gene and characterized by progressive muscle degeneration and weakness in all skeletal muscles. FSHD particularly affects skeletal muscles of the face, shoulders, upper arms, and lower legs. IBM is an inflammatory muscle disease that mainly affects muscles of the thighs and muscles of the arms that control finger and wrist flexion. ALS is a motor neuron disease characterized by stiff muscles, muscle twitching, and muscle atrophy throughout the body due to the degeneration of the motor neurons. Efforts to improve treatment and survival of subjects having these devastating muscle diseases have not been successful.

Healthy bone undergoes a constant remodeling that involves both bone breakdown and bone growth. Bone growth is mediated by the osteoblast cell type whereas the osteoclasts resorb the bone. Pathology occurs when these systems fall out of balance either through downregulation of the anabolic program, upregulation of the catabolic system or a combination of both, resulting in a net bone loss. Therefore, controlling the balance in bone remodeling can be useful for promoting the healing of damage to bone as well as the treatment of disorders, such as osteoporosis, associated with loss of bone mass and bone demineralization.

Bone damage can result from a range of root causes, including age- or cancer-related bone loss, genetic conditions, or adverse side effects of drug treatment. The World Health Organization estimates that osteoporosis alone affects 75 million people in the U.S., Europe, and Japan, and is a significant risk factor in bone damage. In general, the whole of bone loss represents pathological states for which there are few effective treatments. Treatment instead focuses on immobilization, exercise, and dietary modifications rather than agents that directly promote bone growth and increase bone density. With respect to osteoporosis, estrogen, calcitonin, osteocalcin with vitamin K, or high doses of dietary calcium are all used as therapeutic interventions. Other therapeutic approaches to osteoporosis include bisphosphonates, parathyroid hormone, parathyroid hormone related protein, calcimimetics, statins, anabolic steroids, lanthanum and strontium salts, and sodium fluoride. Such therapeutics, however, are often associated with undesirable side effects.

Fibrosis is the formation of excess connective tissue in an organ or tissue. The connective tissue, which can form in response to damage (e.g., injury) or as part of an immune response (e.g., an inflammatory response), can disrupt the structure and function of the organ or tissue in which it forms, leading to an increase in tissue stiffness. Fibrosis can occur in many organs and tissues within the body, including the lung (e.g., pulmonary fibrosis, cystic fibrosis), liver (e.g., cirrhosis), heart (e.g., endomyocardial fibrosis or fibrosis after myocardial infarction), brain (e.g., glial scar formation), skin (e.g., formation of keloids), kidney (e.g., renal fibrosis), and eye (e.g., corneal fibrosis), among others; and is known to be associated with certain medical treatments (e.g., chemotherapy, radiation therapy, and surgery). There are limited treatment options for patients with fibrosis, and most treatments are focused on improving quality of life or temporarily slowing disease progression.

Anemia is a global health problem with health implications that affect both morbidity and mortality. In the United States alone, the prevalence of anemia nearly doubled from 2003 to 2012. Symptoms of anemia include fatigue, weakness, shortness of breath, heart palpitations, and reduced cognitive performance, and children, pregnant women, women of reproductive age, and the elderly have been found to have the highest risk of developing anemia. The most common form of anemia is iron deficiency anemia, but anemia can also be caused by chronic diseases, blood loss, and red blood cell destruction. While iron deficiency anemia can be treated with iron supplements, many other forms of anemia, such as aplastic anemia, anemia of chronic disease, and hemolytic anemia may require blood transfusions.

Thrombocytopenia is a condition characterized by abnormally low levels of platelets, also called thrombocytes, in the blood, and occurs when the bone marrow makes too few platelets or when too many platelets are destroyed or accumulate within an enlarged spleen. Patients with thrombocytopenia may experience internal or external bleeding, bleeding under the skin, and/or bruising. Treatment for thrombocytopenia depends on its cause and severity and is primarily focused on preventing death or disability caused by bleeding. Certain types of thrombocytopenia (e.g., immune thrombocytopenia) may be treated using corticosteroids, but other types of thrombocytopenia may require splenectomy or platelet transfusion.

Neutropenia is a condition characterized by an abnormally low number of neutrophils in the blood. Neutrophils typically constitute 45% to 75% of all white blood cells in the bloodstream and serve as the primary defense against infections. Reduced numbers of neutrophils can lead to difficulty in controlling infections and increase the risk of dying from an infection. In patients with severe neutropenia, infections can rapidly become severe or fatal. Antibiotics are used treat infection in patients having neutropenia, but treatments for neutropenia itself are limited, and primarily involve the use of growth factors, such as colony stimulating factors, to stimulate the production of white blood cells. Blood transfusions have not proven effective.

Myelodysplastic syndromes, or MDS, is a collection of bone marrow disorders characterized by ineffective hematopoiesis, often with a dramatic expansion of progenitor cells that are unable to mature into functioning blood cells. In the United States, there are 60,000 to 170,000 patients with MDS and 15,000 to 20,000 new cases of MDS reported each year. MDS predominantly affects older adults, with approximately 75% of patients aged 60 years or older at diagnosis. Median survival ranges from approximately nine years for very low-risk patients to less than a year for high-risk patients. Anemia is the most frequent consequence of ineffective hematopoiesis in patients with MDS due to low red blood cell production and impacts 90% of MDS patients. Another consequence is thrombocytopenia. Patients with MDS-associated anemia are generally treated with red blood cell transfusions and erythropoiesis stimulating agents (ESAs), which are not approved for such treatment. MDS-associated thrombocytopenia is treated with platelet transfusions and platelet-stimulating agents.

Myelofibrosis is an uncommon type of bone marrow cancer that disrupts the normal production of blood cells. It can cause extensive scarring in the bone marrow, leading to severe anemia and a low number of platelets. Symptoms of myelofibrosis include fatigue, bone pain, easy bruising, easy bleeding, and fever. Patients with aggressive or high-risk myelofibrosis may require a blood transfusion or bone marrow transplant. Other treatment options include therapies that have known risks, such as androgen therapy and treatment with thalidomide or related medications. For patients with intermediate-risk myelofibrosis, treatment is typically directed at symptom management.

Pulmonary hypertension (PH) is a serious condition characterized by higher than normal pressure in the blood vessels between the lungs and the heart. PH can be categorized into five major types: arterial (PAH), venous (PH secondary to left-sided heart disease), hypoxic (PH caused by lung disease), thromboembolic (PH caused by chronic arterial obstruction, e.g., blood clots), or miscellaneous (PH with unclear or multifactorial mechanisms), also known as WHO groups I-V. PAH features increased pressure in blood vessels of the lungs caused by obstruction in or narrowing of small blood vessels in the lungs due to scarring. This leads to increased resistance to blood flow through the lungs and forces the right side of the heart to work harder, which may lead to heart failure, reduced blood oxygenation, and reduced life expectancy. PAH can be idiopathic (e.g., having no identifiable cause), heritable (e.g., familial, often due to a genetic mutation), or may be related to drug use (e.g., methamphetamine or cocaine use), infection (e.g., HIV infection or schistosomiasis), cirrhosis of the liver, congenital heart abnormalities, or connective tissue/autoimmune disorders (e.g., scleroderma or lupus). Treatments for PH include vasodilators, anticoagulants, and supplemental oxygen, but these treatments manage disease symptoms rather than targeting the biological mechanisms that cause the disease.

Excess body weight is an increasing problem in large parts of the world, with about 39% of adults aged 18 years and over found to be overweight in 2016 and about 13% of the world's adult population found to be obese. Increased visceral and subcutaneous fat causes dysfunction of various organs. Excessive body weight is a risk factor for an array of complications, including diabetes (e.g., Type 1 and Type 2 diabetes), cardiovascular disease, and several forms of cancer. Insulin resistance is also associated with obesity and results in pancreatic tissues producing an elevated amount of insulin. Once pancreatic β cells can no longer produce sufficient insulin to meet the demand, hyperglycemia occurs and Type 2 diabetes develops. Adipocytes, which are increased in obesity, are believed to play a role in this process. Despite the prevalence of obesity and metabolic diseases few therapeutic options are available.

Erythropoietin (EPO) is a hormone secreted primarily by the kidney, typically in response to low oxygen levels. It is known to promote the production of erythrocytes, but has also been found to stimulate the mobilization, proliferation, and differentiation of endothelial progenitor cells, to improve gastrointestinal dysmotility, to have broad neuroprotective and anti-inflammatory capabilities, and to play a central role in tissue protection and restoration. Recombinant EPO and EPO mimetics, such as epoetin alfa and epoetin beta, which are often referred to as erythropoiesis-stimulating agents (ESAs), are currently used to treat anemia associated with chronic kidney disease and anemia associated with cancer or chemotherapy. However, recombinant EPO therapy requires intravenous administration one to three times per week for up to twelve weeks, a treatment regimen that is inconvenient for the patient. In addition, treatment with high doses of EPO may lead to the proliferation of cancer cells in subjects with cancer or to tumor recurrence in subjects who have previously had cancer.

There exists a need for novel and effective treatments for muscular diseases, bone diseases, anemia, thrombocytopenia, neutropenia, myelofibrosis, a myelodysplastic syndrome, fibrosis, PH, and metabolic diseases and a need for alternative therapies to EPO.

### SUMMARY OF THE INVENTION

The present invention features polypeptides that include an extracellular activin receptor type II (ActRII) chimera. In some embodiments, a polypeptide of the invention includes an extracellular ActRII chimera fused to the N- or C-terminus of an Fc domain monomer, an Fc domain, or another moiety. Such moieties may be attached by amino acid or other covalent bonds and may increase stability of the polypeptide. A polypeptide including an extracellular ActRII chimera fused to an Fc domain monomer may also form a dimer (e.g., a homodimer or heterodimer) through the interaction between two Fc domain monomers. The polypeptides of the invention may be used to increase lean mass, muscle mass, and/or strength in a subject having or at risk of developing a disease or condition involving weakness or atrophy of muscles, e.g., a neuromuscular disease, sarcopenia, cachexia, disuse atrophy; treatment related muscle loss or atrophy, hypotonia, hypoxia, or muscle loss or atrophy associated with a burn injury. The polypeptides of the invention may also be used to increase bone mass or bone mineral density in a subject having or at risk of developing a disease or condition involving bone damage, e.g., osteoporosis (e.g., primary osteoporosis or secondary osteoporosis), osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss (e.g., bone loss associated with a burn injury), anorexia-related bone loss, diet-related bone loss, bone loss associated with the treatment of obesity, low gravity-related bone loss, or immobility. Additionally, the polypeptides of the invention may be used to increase red blood cell levels (e.g., increase hemoglobin levels, increase hematocrit, and/or increase red blood cell count), promote or increase the maturation and/or differentiation of erythroid progenitors, increase late-stage erythroid precursor maturation, or recruit early-stage progenitors into the erythroid lineage in a subject in need thereof, e.g., a subject having or at risk of developing anemia or blood loss, to increase platelet levels (e.g., increase platelet count) in a subject in need thereof, e.g., a subject having or at risk of developing thrombocytopenia, to increase neutrophil levels (e.g., increase neutrophil count) in a subject in need thereof, e.g., a subject having or at risk of developing neutropenia, to prevent or reduce fibrosis in a subject having or at risk of developing fibrosis, or to treat, prevent, or delay the development or progression of pulmonary hypertension in a subject having or at risk of developing pulmonary hypertension (e.g., arterial, venous, hypoxic, thromboembolic, or miscellaneous pulmonary hypertension). The polypeptides of the invention may also be used to reduce body weight, reduce body fat, increase glucose clearance, increase insulin sensitivity, or reduce fasting insulin levels in a subject having or at risk of developing a metabolic disease, e.g., obesity, Type 1 diabetes, or Type 2 diabetes. The polypeptides of the invention may also be used in the place of EPO to treat a subject having a disease or condition that can be treated with EPO or an ESA, such as end-stage renal disease, renal insufficiency, polycythemia, a disease associated with a dysfunction of endothelial progenitor cells, a neurological disorder or inflammatory brain disease, gastrointestinal dysmotility, ischemia (e.g., central nervous system (CNS), liver, renal, or cardiac ischemia), hypoxia, or a disease or condition having an inflammatory or autoimmune component, to treat a subject receiving kidney dialysis, a subject who is going to undergo surgery, or a subject who has recently received a stem cell transplant, or to increase EPO levels and/or EPO receptor levels in a subject in need thereof. Further, the polypeptides of the invention may also be used to affect myostatin, activin (e.g., activin A and/or activin B), and/or bone morphogenetic protein 9 (BMP9) signaling in a subject having or at risk of developing a disease or condition described herein, such as a disease or condition involving weakness or atrophy of muscles, bone damage or bone demineralization, low blood cell levels (e.g., low hemoglobin levels, low hematocrit, and/or low red blood cell counts), low platelet levels (e.g., low platelet counts), low neutrophil levels (e.g., low neutrophil counts), fibrosis, pulmonary hypertension (e.g., arterial, venous, hypoxic, thromboembolic, or miscellaneous pulmonary hypertension), or a metabolic disease (e.g., obesity, Type 1 diabetes, or Type 2 diabetes).

Exemplary embodiments of the invention are described in the enumerated paragraphs below.
E1. A polypeptide containing an extracellular activin receptor type II (ActRII) chimera, the chimera having a sequence of X₁β₁X₂β₂X₃β₃X₄β₄X₅β₅X₆β₆X₇β₇X₈, wherein:
   X₁ is GAILGRSETQ (X₁ₐ) (SEQ ID NO: 1) or GRGEAETR (X_{1b}) (SEQ ID NO: 2);
   β₁ is ECLFF (β₁ₐ) (SEQ ID NO: 3) or ECIYY (β_{1b}) (SEQ ID NO: 4);
   X₂ is NANWEKDRTN (X₂ₐ) (SEQ ID NO: 5) or (X₂) NANWELERTN (SEQ ID NO: 6);
   β₂ is QTGVEPC (β₂ₐ) (SEQ ID NO: 7) or QSGLERC (β_{2b}) (SEQ ID NO: 8);
   X₃ is YGDKDKR (X₃ₐ) (SEQ ID NO: 9) or (X_{3b}) EGEQDKR (SEQ ID NO: 10);
   β₃ is RHCFATWKNI (β₃ₐ) (SEQ ID NO: 11) or a portion thereof that comprises HCFATWK (SEQ ID NO: 12) or LHCYASWRNS (β_{3b}) (SEQ ID NO: 13) or a portion thereof that comprises HCYASWR (SEQ ID NO: 14), wherein when β₃ is HCFATWK or HCYASWR, the chimera comprises contiguous amino acids from ActRIIA or ActRIIB connecting β₃ to X₃ and X₄; X₄ is SG;
   β₄ is SIEIVKQGCW (β₄ₐ) (SEQ ID NO: 15) or a portion thereof that comprises EIVKQGCW (SEQ ID NO: 16) or TIELVKKGCW (β_{4b}) (SEQ ID NO: 17) or a portion thereof that comprises ELVKKGCW (SEQ ID NO: 18), wherein when β₄ is EIVKQGCW or ELVKKGCW, the chimera comprises contiguous amino acids from ActRIIA or ActRIIB connecting β₄ to X₄;
   X₅ is LDDINCYDRTDC (X₅ₐ) (SEQ ID NO: 19) or LDDFNCYDRQEC (X_{5b}) (SEQ ID NO: 20);
   β₅ is VEK (β₅ₐ) or a portion thereof that comprises VE or VAT (β_{5b}) or a portion thereof that comprises V, wherein when β₅ is VE or V, the chimera comprises contiguous amino acids from ActRIIA or ActRIIB connecting β₅ to X₆;
   X₆ is KDSPEV (X₆ₐ) (SEQ ID NO: 21) or EENPQV (X₆) (SEQ ID NO: 22); β6 is YFCCCE (SEQ ID NO: 23);
   X₇ is GNMCNE (X₇ₐ) (SEQ ID NO: 24) or GNFCNE (X_{7b}) (SEQ ID NO: 25);
   β₇ is KFSYF (β₇ₐ) (SEQ ID NO: 26) or a portion thereof that comprises SYF or RFTHL (β_{7b}) (SEQ ID NO: 27) or a portion thereof that comprises T, wherein when β₇ is SYF or T, the chimera comprises contiguous amino acids from ActRIIA or ActRIIB connecting β₇ to X₇ and X₈; and
   X₈ is PEMEVTQPTS (X₈ₐ) (SEQ ID NO: 28) or PEAGGPEVTYEPPPTAPT (X_{8b}) (SEQ ID NO: 29), wherein:
      (i) at least one of Bia, β₂ₐ, β₃ₐ, β₄ₐ, β₅ₐ, or β₇a and at least one of β_{1b}, β_{2b}, β_{3b}, β_{4b}, β_{5b}, or β_{7b} is present in the chimera, and/or
      (ii) at least one of X₁ₐ, X₂ₐ, X₃ₐ, X₅ₐ, X₆ₐ, X₇ₐ, or X₈ₐ and at least one of X_{1b}, X_{2b}, X_{3b}, X_{5b}, X_{6b}, X_{7b}, or X_{8b} is present in the chimera,
      optionally wherein the chimera is truncated from the N-terminus by deletion of 1, 2, 3, 4, 5, 6, 7, 8, or 9 amino acids, wherein the chimera retains the two amino acids before the first cysteine.
E2. The polypeptide of E1, wherein X₁ is GAILGRSETQ (X₁ₐ).
E3. The polypeptide of E1, wherein X₁ is GRGEAETR (X_{1b}).
E4. The polypeptide of any one of E1-E3, wherein β₁ is ECLFF (β₁ₐ).
E5. The polypeptide of any one of E1-E3, wherein β₁ is ECIYY (β_{1b}).
E6. The polypeptide of any one of E1-E5, wherein X₂ is NANWEKDRTN (X₂ₐ).
E7. The polypeptide of any one of E1-E5, wherein X₂ is NANWELERTN (X₂).
E8. The polypeptide of any one of E1-E7, wherein β₂ is QTGVEPC (β₂ₐ).
E9. The polypeptide of any one of E1-E7, wherein β₂ is QSGLERC (β_{2b}).
E10. The polypeptide of any one of E1-E9, wherein X₃ is YGDKDKR (X₃ₐ).
E11. The polypeptide of any one of E1-E9, wherein X₃ is EGEQDKR (X_{3b}).
E12. The polypeptide of any one of E1-E11, wherein β₃ is RHCFATWKNI (β₃ₐ).
E13. The polypeptide of any one of E1-E11, wherein β₃ is LHCYASWRNS (β_{3b}).
E14. The polypeptide of any one of E1-E11, wherein β₃ is HCFATWK, wherein the chimera comprises contiguous amino acids from ActRIIA or ActRIIB connecting β₃ to X₃ and X₄.
E15. The polypeptide of any one of E1-E11, wherein β₃ is HCYASWR, wherein the chimera comprises contiguous amino acids from ActRIIA or ActRIIB connecting β₃ to X₃ and X₄.
E16. The polypeptide of E14 or E15, wherein the contiguous amino acids connecting β₃ to X₃ are from ActRIIA.
E17. The polypeptide of E14 or E15, wherein the contiguous amino acids connecting β₃ to X₃ are from ActRIIB.
E18. The polypeptide of any one of E14-E17, wherein the contiguous amino acids connecting β₃ to X₄ are from ActRIIA.
E19. The polypeptide of any one of E14-E17, wherein the contiguous amino acids connecting β3 to X₄ are from ActRIIB.
E20. The polypeptide of any one of E1-E19, wherein β₄ is SIEIVKQGCW (β₄ₐ).
E21. The polypeptide of any one of E1-E19, wherein β4 is TIELVKKGCW (β_{4b}).
E22. The polypeptide of any one of E1-E19, wherein β₄ is EIVKQGCW, wherein the chimera comprises contiguous amino acids from ActRIIA or ActRIIB connecting β4 to X₄.
E23. The polypeptide of any one of E1-E19, wherein β₄ is ELVKKGCW, wherein the chimera comprises contiguous amino acids from ActRIIA or ActRIIB connecting 4 to X₄.
E24. The polypeptide of E22 or E23, wherein the contiguous amino acids connecting β₄ to X₄ are from ActRIIA.
E25. The polypeptide of E22 or E23, wherein the contiguous amino acids connecting β₄ to X₄ are from ActRIIB.
E26. The polypeptide of any one of E1-E25, wherein X₅ is LDDINCYDRTDC (X₅ₐ).
E27. The polypeptide of any one of E1-E25, wherein X₅ is LDDFNCYDRQEC (X_{5b}).
E28. The polypeptide of any one of E1-E27, wherein β₅ is VEK (β₅ₐ).
E29. The polypeptide of any one of E1-E27, wherein β₅ is VAT (β_{5b}).
E30. The polypeptide of any one of E1-E27, wherein β₅ is VE, wherein the chimera comprises contiguous amino acids from ActRIIA or ActRIIB connecting β5 to X₆.
E31. The polypeptide of any one of E1-E27, wherein β₃ is V, wherein the chimera comprises contiguous amino acids from ActRIIA or ActRIIB connecting β₅ to X₆.
E32. The polypeptide of E30 or E31, wherein the contiguous amino acids connecting β₅ to X₆ are from ActRIIA.
E33. The polypeptide of E30 or E31, wherein the contiguous amino acids connecting β₅ to X₆ are from ActRIIB.
E34. The polypeptide of any one of E1-E33, wherein X₆ is KDSPEV (X₆ₐ).
E35. The polypeptide of any one of E1-E33, wherein X₆ is EENPQV (X_{6b}).
E36. The polypeptide of any one of E1-E35, wherein X₇ is GNMCNE (X₇ₐ).
E37. The polypeptide of any one of E1-E35, wherein X₇ is GNFCNE (X_{7b}).
E38. The polypeptide of any one of E1-E37, wherein β₇ is KFSYF (β₇ₐ).
E39. The polypeptide of any one of E1-E37, wherein β₇ is RFTHL (β_{7b}).
E40. The polypeptide of any one of E1-E37, wherein β₇ is SYF, wherein the chimera comprises contiguous amino acids from ActRIIA or ActRIIB connecting β₇ to X₇.
E41. The polypeptide of any one of E1-E37, wherein β₇ is T, wherein the chimera comprises contiguous amino acids from ActRIIA or ActRIIB connecting β₇ to X₇ and X₈.
E42. The polypeptide of E40 or E41, wherein the contiguous amino acids connecting β₇ to X₇ are from ActRIIA.
E43. The polypeptide of E40 or E41, wherein the contiguous amino acids connecting β₇ to X₇ are from ActRIIB.
E44. The polypeptide of any one of E41-E43, wherein the contiguous amino acids connecting β₇ to X₈ are from ActRIIA.
E45. The polypeptide of any one of E41-E43, wherein the contiguous amino acids connecting β₇ to X₈ are from ActRIIB.
E46. The polypeptide of any one of E1-E45, wherein X₈ is PEMEVTQPTS (X₈ₐ).
E47. The polypeptide of any one of E1-E45, wherein X₈ is PEAGGPEVTYEPPPTAPT (X₈).
E48. The polypeptide of E1, wherein the ActRII chimera has the sequence of any one of SEQ ID NOS: 96-126.
E49. The polypeptide of any one of E1-E48, wherein the chimera is truncated from the N-terminus by deletion of one amino acid.
E50. The polypeptide of any one of E1-E48, wherein the chimera is truncated from the N-terminus by deletion of two amino acids.
E51. The polypeptide of any one of E1-E48, wherein the chimera is truncated from the N-terminus by deletion of three amino acids.
E52. The polypeptide of any one of E1-E48, wherein the chimera is truncated from the N-terminus by deletion of four amino acids.
E53. The polypeptide of any one of E1-E48, wherein the chimera is truncated from the N-terminus by deletion of five amino acids.
E54. The polypeptide of any one of E1-E48, wherein the chimera is truncated from the N-terminus by deletion of six amino acids.
E55. The polypeptide of any one of E1-E48, wherein the chimera is truncated from the N-terminus by deletion of seven amino acids.
E56. The polypeptide of any one of E1-E48, wherein the chimera is truncated from the N-terminus by deletion of eight amino acids.
E57. The polypeptide of any one of E1-E48, wherein the chimera is truncated from the N-terminus by deletion of nine amino acids.
E58. The polypeptide of any one of E1-E57, wherein the chimera further includes a C-terminal extension of one or more amino acids (e.g., 1, 2, 3, 4, 5, 6, or more amino acids from the same position in wild-type extracellular ActRIIA or ActRIIB).
E59. The polypeptide of E58, wherein the C-terminal extension is NP.
E60. The polypeptide of E58, wherein the C-terminal extension is NPVTPK (SEQ ID NO: 91).
E61. The polypeptide of any one of E1-E60, wherein the polypeptide further includes an Fc domain monomer fused to the C-terminus of the polypeptide (e.g., the C-terminus of the chimera) by way of a linker.
E62. The polypeptide of E61, wherein the Fc domain monomer has the sequence of SEQ ID NO: 34.
E63. The polypeptide of E61 or E62, wherein the polypeptide forms a dimer (e.g., a dimer formed by two Fc domain monomers).
E64. The polypeptide of any one of E1-E60, wherein the polypeptide further includes an Fc domain fused to the C-terminus of the polypeptide (e.g., the C-terminus of the chimera) by way of a linker.
E65. The polypeptide of E64, wherein the Fc domain has the sequence of SEQ ID NO: 87.
E66. The polypeptide of E64, wherein the Fc domain has the sequence of SEQ ID NO: 35.
E67. The polypeptide of E64, wherein the Fc domain does not form a dimer.
E68. The polypeptide of any one of E1-E60, wherein the polypeptide further includes an albumin-binding peptide fused to the C-terminus of the polypeptide (e.g., the C-terminus of the chimera) by way of a linker.
E69. The polypeptide of E68, wherein the albumin-binding peptide has the sequence of SEQ ID NO: 88.
E70. The polypeptide of any one of E1-E60, wherein the polypeptide further includes a fibronectin domain fused to the C-terminus of the polypeptide (e.g., the C-terminus of the chimera) by way of a linker.
E71. The polypeptide of E70, wherein the fibronectin domain has the sequence of SEQ ID NO: 89.
E72. The polypeptide of any one of E1-E60, wherein the polypeptide further includes a human serum albumin fused to the C-terminus of the polypeptide (e.g., the C-terminus of the chimera) by way of a linker.
E73. The polypeptide of E72, wherein the human serum albumin has the sequence of SEQ ID NO: 90.
E74. The polypeptide of any one of E61-E73, wherein the linker is an amino acid spacer.
E75. The polypeptide of E74, wherein the amino acid spacer is GGG, GGGA (SEQ ID NO: 36), GGGG (SEQ ID NO: 38), GGGAG (SEQ ID NO: 68), GGGAGG (SEQ ID NO: 69), GGGAGGG (SEQ ID NO: 70), GA, GS, GG, GGA, GGS, GGG, GGGS (SEQ ID NO: 37), GGGGA (SEQ ID NO: 39), GGGGS (SEQ ID NO: 40), GGGGG (SEQ ID NO: 41), GGAG (SEQ ID NO: 42), GGSG (SEQ ID NO: 43), AGGG (SEQ ID NO: 44), SGGG (SEQ ID NO: 45), GAGA (SEQ ID NO: 46), or GSGS (SEQ ID NO: 47).
E76. The polypeptide of E75, wherein the amino acid spacer is GGG.
E77. The polypeptide of E75, wherein the amino acid spacer is GGGS (SEQ ID NO: 37).
E78. The polypeptide of E75, wherein the amino acid spacer is GGS.
E79. The polypeptide of E74, wherein the amino acid spacer is GAGAGA (SEQ ID NO: 48), GSGSGS (SEQ ID NO: 49), GAGAGAGA (SEQ ID NO: 50), GSGSGSGS (SEQ ID NO: 51), GAGAGAGAGA (SEQ ID NO: 52), GSGSGSGSGS (SEQ ID NO: 53), GAGAGAGAGAGA (SEQ ID NO: 54), GSGSGSGSGSGS (SEQ ID NO: 55), GGAGGA (SEQ ID NO: 56), GGSGGS (SEQ ID NO: 57), GGAGGAGGA (SEQ ID NO: 58), GGSGGSGGS (SEQ ID NO: 59), GGAGGAGGAGGA (SEQ ID NO: 60), and GGSGGSGGSGGS (SEQ ID NO: 61), GGAGGGAG (SEQ ID NO: 62), GGSGGGSG (SEQ ID NO: 63), GGAGGGAGGGAG (SEQ ID NO: 64), and GGSGGGSGGGSG (SEQ ID NO: 65), GGGGAGGGGAGGGGA (SEQ ID NO: 66), GGGGSGGGGSGGGGS (SEQ ID NO: 67), AAAL (SEQ ID NO: 71), AAAK (SEQ ID NO: 72), AAAR (SEQ ID NO: 73), EGKSSGSGSESKST (SEQ ID NO: 74), GSAGSAAGSGEF (SEQ ID NO: 75), AEAAAKEAAAKA (SEQ ID NO: 76), KESGSVSSEQLAQFRSLD (SEQ ID NO: 77), GENLYFQSGG (SEQ ID NO: 78), SACYCELS (SEQ ID NO: 79), RSIAT (SEQ ID NO: 80), RPACKIPNDLKQKVMNH (SEQ ID NO: 81), GGSAGGSGSGSSGGSSGASGTGTAGGTGSGSGTGSG (SEQ ID NO: 82), AAANSSIDLISVPVDSR (SEQ ID NO: 83), GGSGGGSEGGGSEGGGSEGGGSEGGGSEGGGSGGGS (SEQ ID NO: 84), EAAAK (SEQ ID NO: 85), or PAPAP (SEQ ID NO: 86).
E80. The polypeptide of any one of E1-E79, wherein the polypeptide (e.g., an ActRII chimera-Fc fusion protein) has a serum half-life of at least seven days.
E81. The polypeptide of any one of E1-E80, wherein the polypeptide has increased binding to one or more an ActRII ligands (e.g., activin A, activin B, myostatin, and/or GDF-11) compared to wild-type ActRIIA and/or wild-type ActRIIB (e.g., wild-type extracellular ActRIIA and/or ActRIIB).
E82. The polypeptide of any one of E1-E81, wherein the polypeptide has decreased binding to BMP9 compared to wild-type ActRIIB (e.g., wild-type extracellular ActRIIB, e.g., the polypeptide binds to BMP9 with a K_{D} of about 40 pM or higher (e.g., 45, 50, 60, 70, 80, 90, 100, 200, 300, 400, 500, 600, 700, 800, or 900 pM or about 1, 2, 3, 4, 5, 6, 7, 8, 9, 10, 15, 20, 25, 30, 35, 40, 45, 50, 55, 60, 65, 70, 75, or 80 nM or higher, e.g., a K_{D} of 1 nM or higher).
E83. The polypeptide of any one of E1-E82, wherein the polypeptide binds to activin A, activin B, and/or myostatin and has reduced or weak binding to human BMP9 (e.g., compared to wild-type ActRIIB).
E84. The polypeptide of any one of E81-E83, wherein the polypeptide does not substantially bind to human BMP9.
E85. The polypeptide of any one of E1-E84, wherein the polypeptide binds to human activin A with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 300 PM and about 1 pM).
E86. The polypeptide of any one of E1-E85, wherein the polypeptide binds to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 200 pM and about 1 pM, or a K_{D} of less than 1 pM).
E87. The polypeptide of any one of E1-E86, wherein the polypeptide binds to human GDF-11 with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 200 pM and about 1 pM, or a K_{D} of less than 1 pM).
E88. The polypeptide of any one of E1-E87, wherein the polypeptide binds to human GDF-8 with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 5 pM).
E89. The polypeptide of any one of E1-E88, wherein the polypeptide binds to human BMP10 with a K_{D} of about 1 pM or higher (e.g., a K_{D} of about 1, 5, 15, 30, 25, 30, 35, 40, 45, 50, 60, 70, 80, 90, 100, 200, 300, 400, 500, 600, 700, 800, or 900 pM or about 1, 2, 3, 4, 5, 6, 7, 8, 9, or 10 nM or higher).
E90. A nucleic acid molecule encoding a polypeptide of any one of E1-E89.
E91. A vector including the nucleic acid molecule of E90.
E92. A host cell that expresses the polypeptide of any one of E1-E89, wherein the host cell includes the nucleic acid molecule of E90 or the vector of E91, wherein the nucleic acid molecule or vector is expressed in the host cell.
E93. A method of preparing the polypeptide of any one of E1-E89, wherein the method includes: a) providing a host cell containing the nucleic acid molecule of E90 or the vector of E91, and b) expressing the nucleic acid molecule or vector in the host cell under conditions that allow for the formation of the polypeptide.
E94. A pharmaceutical composition including the polypeptide of any one of E1-E89, the nucleic acid molecule of E90, or the vector of E91, and one or more pharmaceutically acceptable carriers or excipients.
E95. The pharmaceutical composition of E94, wherein the polypeptide, nucleic acid molecule, or vector is in a therapeutically effective amount.
E96. A construct including two identical polypeptides (e.g., a homodimer), each including the polypeptide of any one of E1-E60 fused to the N- or C-terminus of an Fc domain monomer (e.g., the sequence of SEQ ID NO: 34). The two Fc domain monomers in the two polypeptides interact to form an Fc domain in the construct.
E97. A construct including two different polypeptides (e.g., a heterodimer) each including the polypeptide of any one of E1-E60 fused to the N- or C-terminus of an Fc domain monomer (e.g., the sequence of SEQ ID NO: 34). The two Fc domain monomers in the two polypeptides interact to form an Fc domain in the construct.
E98. A method of increasing lean mass in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E99. A method of increasing muscle mass in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E100. A method of increasing muscle strength in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E101. The method of any one of E98-E100, wherein the subject has or is at risk of developing a neuromuscular disease, sarcopenia, cachexia, disuse atrophy, treatment-related muscle loss or atrophy, hypotonia, muscle loss or atrophy associated with hypoxia, or muscle loss or atrophy associated with a burn injury.
E102. A method of treating a subject having or at risk of developing a muscle disease (e.g., a disease or condition involving muscle weakness or atrophy) by administering to the subject a therapeutically effective amount of the polypeptide of any one of E1-E89, the nucleic acid molecule of E90, the vector of E91, the construct of E96 or E97, or the pharmaceutical composition of E94 or E95.
E103. The method of E102, wherein the muscle disease is a neuromuscular disease, sarcopenia, cachexia, disuse atrophy, treatment-related muscle loss or atrophy, hypotonia, muscle loss or atrophy associated with hypoxia, or muscle loss or atrophy associated with a burn injury.
E104. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing a disease or condition involving weakness and/or atrophy of muscles by administering to the subject a therapeutically effective amount of the polypeptide of any one of E1-E89, the nucleic acid molecule of E90, the vector of E91, the construct of E96 or E97, or the pharmaceutical composition of E94 or E95.
E105. The method of E104, wherein the disease or condition is a neuromuscular disease, sarcopenia, cachexia, disuse atrophy, treatment-related muscle loss or atrophy, hypotonia, muscle loss or atrophy associated with hypoxia, or muscle loss or atrophy associated with a burn injury.
E106. A method of treating a subject having or at risk of developing a neuromuscular disease, comprising administering to the subject a therapeutically effective amount of the polypeptide of any one of E1-E89, the nucleic acid molecule of E90, the vector of E91, the construct of E96 or E97, or the pharmaceutical composition of E94 or E95.
E107. The method of any one of E101, E103, and E105, wherein the subject has or is at risk of developing a neuromuscular disease or wherein the disease or condition is a neuromuscular disease.
E108. A method of treating a subject having or at risk of developing DMD by administering to the subject a therapeutically effective amount of the polypeptide of any one of E1-E89, the nucleic acid molecule of E90, the vector of E91, the construct of E96 or E97, or the pharmaceutical composition of E94 or E95.
E109. A method of treating a subject having or at risk of developing FSHD by administering to the subject a therapeutically effective amount of the polypeptide of any one of E1-E89, the nucleic acid molecule of E90, the vector of E91, the construct of E96 or E97, or the pharmaceutical composition of E94 or E95.
E110. A method of treating a subject having or at risk of developing IBM by administering to the subject a therapeutically effective amount of the polypeptide of any one of E1-E89, the nucleic acid molecule of E90, the vector of E91, the construct of E96 or E97, or the pharmaceutical composition of E94 or E95.
E111. A method of treating a subject having or at risk of developing ALS by administering to the subject a therapeutically effective amount of the polypeptide of any one of E1-E89, the nucleic acid molecule of E90, the vector of E91, the construct of E96 or E97, or the pharmaceutical composition of E94 or E95.
E112. The method of any one of E101, E103, and E105, wherein the subject has or is at risk of developing a sarcopenia or wherein the disease or condition is sarcopenia.
E113. A method of treating a subject having or at risk of developing sarcopenia by administering to the subject a therapeutically effective amount of the polypeptide of any one of E1-E89, the nucleic acid molecule of E90, the vector of E91, the construct of E96 or E97, or the pharmaceutical composition of E94 or E95.
E114. The method of any one of E101, E103, and E105, wherein the subject has or is at risk of developing disuse atrophy or wherein the disease or condition is disuse atrophy.
E115. A method of treating a subject having or at risk of developing disuse atrophy by administering to the subject a therapeutically effective amount of the polypeptide of any one of E1-E89, the nucleic acid molecule of E90, the vector of E91, the construct of E96 or E97, or the pharmaceutical composition of E94 or E95.
E116. The method of any one of E101, E103, and E105, wherein the subject has or is at risk of developing treatment-related muscle loss or atrophy or wherein the disease or condition is treatment-related muscle loss or atrophy.
E117. A method of treating a subject having or at risk of developing treatment-related muscle loss or atrophy by administering to the subject a therapeutically effective amount of the polypeptide of any one of E1-E89, the nucleic acid molecule of E90, the vector of E91, the construct of E96 or E97, or the pharmaceutical composition of E94 or E95.
E118. The method of any one of E101, E103, E105, E116, and E117, wherein the treatment is glucocorticoid treatment, FGF-21 treatment, GLP-1 treatment, treatment with an FGF-21- or GLP-1-containing therapeutic, bariatric surgery (e.g., gastric bypass), cancer therapy (e.g., chemotherapy or radiation), or treatment for obesity or Type 2 diabetes.
E119. The method of any one of E101, E103, and E105, wherein the subject has or is at risk of developing hypotonia or wherein the disease or condition is hypotonia.
E120. A method of treating a subject having or at risk of developing hypotonia by administering to the subject a therapeutically effective amount of the polypeptide of any one of E1-E89, the nucleic acid molecule of E90, the vector of E91, the construct of E96 or E97, or the pharmaceutical composition of E94 or E95.
E121. The method of any one of E101, E103, and E105, wherein the subject has or is at risk of developing muscle loss or atrophy associated with hypoxia or wherein the disease or condition is muscle loss or atrophy associated with hypoxia.
E122. A method of treating a subject having or at risk of developing muscle loss or atrophy associated with hypoxia by administering to the subject a therapeutically effective amount of the polypeptide of any one of E1-E89, the nucleic acid molecule of E90, the vector of E91, the construct of E96 or E97, or the pharmaceutical composition of E94 or E95.
E123. The method of any one of E101, E103, and E105, wherein the subject has or is at risk of developing muscle loss or atrophy associated with a burn injury or wherein the disease or condition is muscle loss or atrophy associated with a burn injury.
E124. A method of treating a subject having or at risk of developing muscle loss or atrophy associated with a burn injury by administering to the subject a therapeutically effective amount of the polypeptide of any one of E1-E89, the nucleic acid molecule of E90, the vector of E91, the construct of E96 or E97, or the pharmaceutical composition of E94 or E95.
E125. The method of any one of E101, E103, and E105, wherein the subject has or is at risk of developing cachexia or wherein the disease or condition is cachexia.
E126. A method of treating a subject having or at risk of developing cachexia by administering to the subject a therapeutically effective amount of the polypeptide of any one of E1-E89, the nucleic acid molecule of E90, the vector of E91, the construct of E96 or E97, or the pharmaceutical composition of E94 or E95.
E127. The method of any one of E101, E103, E105, E125, and E126, wherein the cachexia is cancer cachexia, HIV-related cachexia, cardiac cachexia (e.g., cachexia associated with heart failure), cachexia associated with chronic kidney disease, or pulmonary cachexia (e.g., cachexia associated with COPD).
E128. The method of any one of E98-E127, wherein the method increases muscle mass.
E129. The method of any one of E98-E128, wherein the method increases lean mass.
E130. The method of any one of E98-E129, wherein the method increases muscle strength.
E131. A method of increasing bone mineral density in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E132. A method of reducing bone resorption in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E133. A method of increasing bone formation in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E134. A method of increasing bone strength in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E135. A method of reducing the risk or occurrence of bone fracture in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E136. The method of any one of E131-E135, wherein the subject has or is at risk of developing osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss.
E137. A method of treating a subject having or at risk of developing a bone disease by administering to the subject a therapeutically effective amount of the polypeptide of any one of E1-E89, the nucleic acid molecule of E90, the vector of E91, the construct of E96 or E97, or the pharmaceutical composition of E94 or E95.
E138. The method of E137, wherein the bone disease is osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss.
E139. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing a disease or condition involving bone damage by administering to the subject a therapeutically effective amount of the polypeptide of any one of E1-E89, the nucleic acid molecule of E90, the vector of E91, the construct of E96 or E97, or the pharmaceutical composition of E94 or E95.
E140. The method of E139, wherein the disease or condition is osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss.
E141. The method of any one of E136, E138, and E140, wherein the subject has or is at risk of developing osteoporosis or wherein the disease or condition is osteoporosis.
E142. A method of treating a subject having or at risk of developing osteoporosis by administering to the subject a therapeutically effective amount of the polypeptide of any one of E1-E89, the nucleic acid molecule of E90, the vector of E91, the construct of E96 or E97, or the pharmaceutical composition of E94 or E95.
E143. The method of any one of E136, E138, and E140, wherein the subject has or is at risk of developing osteogenesis imperfecta or wherein the disease or condition is osteogenesis imperfecta.
E144. A method of treating a subject having or at risk of developing osteogenesis imperfecta by administering to the subject a therapeutically effective amount of the polypeptide of any one of E1-E89, the nucleic acid molecule of E90, the vector of E91, the construct of E96 or E97, or the pharmaceutical composition of E94 or E95.
E145. The method of any one of E136, E138, and E140, wherein the subject has or is at risk of developing osteopenia or wherein the disease or condition is osteopenia.
E146. A method of treating a subject having or at risk of developing osteopenia by administering to the subject a therapeutically effective amount of the polypeptide of any one of E1-E89, the nucleic acid molecule of E90, the vector of E91, the construct of E96 or E97, or the pharmaceutical composition of E94 or E95.
E147. The method of any one of E136, E138, and E140, wherein the subject has or is at risk of developing a bone fracture or wherein the disease or condition is bone fracture.
E148. A method of treating a subject having or at risk of developing a bone fracture by administering to the subject a therapeutically effective amount of the polypeptide of any one of E1-E89, the nucleic acid molecule of E90, the vector of E91, the construct of E96 or E97, or the pharmaceutical composition of E94 or E95.
E149. The method of any one of E136, E138, and E140, wherein the subject has or is at risk of developing bone cancer or cancer metastasis-related bone loss or wherein the disease or condition is bone cancer or cancer metastasis-related bone loss.
E150. A method of treating a subject having or at risk of developing bone cancer or cancer metastasis-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E151. The method of any one of E136, E138, and E140, wherein the subject has or is at risk of developing Paget's disease or wherein the disease or condition is Paget's disease.
E152. A method of treating a subject having or at risk of developing Paget's disease by administering to the subject a therapeutically effective amount of the polypeptide of any one of E1-E89, the nucleic acid molecule of E90, the vector of E91, the construct of E96 or E97, or the pharmaceutical composition of E94 or E95.
E153. The method of any one of E136, E138, and E140, wherein the subject has or is at risk of developing renal osteodystrophy or wherein the disease or condition is renal osteodystrophy.
E154. A method of treating a subject having or at risk of developing renal osteodystrophy by administering to the subject a therapeutically effective amount of the polypeptide of any one of
E1-E89, the nucleic acid molecule of E90, the vector of E91, the construct of E96 or E97, or the pharmaceutical composition of E94 or E95.
E155. The method of any one of E136, E138, and E140, wherein the subject has or is at risk of developing treatment-related bone loss or wherein the disease or condition is treatment-related bone loss.
E156. A method of treating a subject having or at risk of developing treatment-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E157. The method of any one of E136, E138, and E140, wherein the subject has or is at risk of developing diet-related bone loss or wherein the disease or condition is diet-related bone loss.
E158. A method of treating a subject having or at risk of developing diet-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E159. The method of any one of E136, E138, and E140, wherein the subject has or is at risk of developing low gravity-related bone loss or wherein the disease or condition is low gravity-related bone loss.
E160. A method of treating a subject having or at risk of developing low gravity-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E161. The method of any one of E136, E138, and E140, wherein the subject has or is at risk of developing immobility-related bone loss or wherein the disease or condition is immobility-related bone loss.
E162. A method of treating a subject having or at risk of developing immobility-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E163. The method of any one of E136, E138, and E140, wherein the subject has or is at risk of developing neuromuscular disease-related bone loss or wherein the disease or condition is neuromuscular disease-related bone loss.
E164. A method of treating a subject having or at risk of developing neuromuscular disease-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E165. The method of any one of E101, E103, E105-E107, E136, E138, E140, E163, and E164, wherein the neuromuscular disease is a muscular dystrophy, amyotrophic lateral sclerosis (ALS), autonomic neuropathy, botulism, Charcot-Marie-Tooth disease (CMT), chronic inflammatory demyelinating polyradiculoneuropathy, congenital myasthenic syndrome, a congenital myopathy, cramp-fasciculation syndrome, dermatomyositis, diabetic neuropathy, a distal myopathy, a dystrophinopathy, an endocrine myopathy, a focal muscular atrophy, glycogen storage disease type II, Guillain-Barre syndrome, hereditary spastic paraplegia, inclusion body myositis (IBM), Isaac's syndrome, Kearns-Sayre syndrome, Kennedy disease, Lambert-Eaton myasthenic syndrome, a metabolic myopathy, a metabolic neuropathy, a mitochondrial myopathy, a motor neuron disease, multiple sclerosis, myasthenia gravis, myotonic dystrophy, a necrotizing myopathy, neuromyotonia, neuropathy of Friedreich's Ataxia, a nutritional neuropathy, peripheral neuropathy, polymyositis, primary lateral sclerosis, Schwartz-Jampel Syndrome, small fiber neuropathy, spinal and bulbar muscular atrophy, spinal muscular atrophy (SMA), spinal muscular atrophy with respiratory distress type 1, stiff person syndrome, toxic neuropathy, or Troyer syndrome.
E166. The method of E165, wherein the neuromuscular disease is a muscular dystrophy.
E167. The method of E166, wherein the muscular dystrophy is Duchenne muscular dystrophy (DMD), facioscapulohumeral muscular dystrophy (FSHD), Becker muscular dystrophy (BMD), myotonic dystrophy (DM), congenital muscular dystrophy, limb-girdle muscular dystrophy (LGMD), distal muscular dystrophy (DD), oculopharyngeal muscular dystrophy (OPMD), or Emery-Dreifuss muscular dystrophy (EDMD).
E168. The method of E167, wherein the muscular dystrophy is DMD.
E169. The method of E167, wherein the muscular dystrophy is FSHD.
E170. The method of E167, wherein the muscular dystrophy is BMD.
E171. The method of E167, wherein the muscular dystrophy is DM.
E172. The method of E167, wherein the muscular dystrophy is LGMD.
E173. The method of E167, wherein the muscular dystrophy is DD.
E174. The method of E167, wherein the muscular dystrophy is OPMD.
E175. The method of E167, wherein the muscular dystrophy is EDMD.
E176. The method of E167, wherein the muscular dystrophy is a congenital muscular dystrophy.
E177. The method of E176, wherein the congenital muscular dystrophy is congenital muscular dystrophy type 1A (MDC1A), congenital muscular dystrophy type 1C (MDC1C), congenital muscular dystrophy type 1D (MDC1D), congenital muscular dystrophy type 1B (MDC1B), Fukuyama congenital muscular dystrophy (FCMD), muscle-eye-brain disease (MEB), Walker-Warburg Syndrome (WWS), rigid spine muscular dystrophy (RSMD1), Ullrich congenital muscular dystrophy (UCMD), or muscular dystrophy associated with a mutation in integrin alpha 7, integrin alpha 9, docking protein 7, laminin A/C, SECIS binding protein 2, or choline kinase beta.
E178. The method of E177, wherein the congenital muscular dystrophy is MDC1A.
E179. The method of E177, wherein the congenital muscular dystrophy is MDC1B.
E180. The method of E177, wherein the congenital muscular dystrophy is MDC1C.
E181. The method of E177, wherein the congenital muscular dystrophy is MDC1D.
E182. The method of E177, wherein the congenital muscular dystrophy is FCMD.
E183. The method of E177, wherein the congenital muscular dystrophy is MEB.
E184. The method of E177, wherein the congenital muscular dystrophy is WWS.
E185. The method of E177, wherein the congenital muscular dystrophy is RSMD1.
E186. The method of E177, wherein the congenital muscular dystrophy is UCMD.
E187. The method of E165, wherein the neuromuscular disease is CMT.
E188. The method of E165, wherein the neuromuscular disease is ALS.
E189. The method of E165, wherein the neuromuscular disease is SMA.
E190. The method of E165, wherein the neuromuscular disease is IBM.
E191. The method of E165, wherein the neuromuscular disease is myasthenia gravis.
E192. The method of E165, wherein the neuromuscular disease is multiple sclerosis.
E193. The method of any one of E136, E138, and E140, wherein the subject has or is at risk of developing burn-induced bone loss or wherein the disease or condition is burn-induced bone loss.
E194. A method of treating a subject having or at risk of developing burn-induced bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E195. The method of any one of E136, E138, and E140, wherein the subject has or is at risk of developing anorexia-related bone loss or wherein the disease or condition is anorexia-related bone loss.
E196. A method of treating a subject having or at risk of developing anorexia-related bone loss by administering to the subject a therapeutically effective amount of the polypeptide of any one of E1-E89, the nucleic acid molecule of E90, the vector of E91, the construct of E96 or E97, or the pharmaceutical composition of E94 or E95.
E197. The method of any one of E136, E138, and E140-E142, wherein the osteoporosis is primary osteoporosis.
E198. The method of E197, wherein the primary osteoporosis is age-related osteoporosis or hormone-related osteoporosis.
E199. The method of any one of E136, E138, and E140-E142-E161, wherein the osteoporosis is secondary osteoporosis.
E200. The method of E199, wherein the secondary osteoporosis is immobilization-induced osteoporosis or glucocorticoid-induced osteoporosis, or results from an endocrinopathy, a gastrointestinal disorder, a hematological disorder, an autoimmune disorder, renal disease, a medication, alcoholism, or transplantation.
E201. The method of any one of E136, E138, E140, E149, and E140, wherein cancer is multiple myeloma.
E202. The method of any one of E136, E138, 140, E155, and E156, wherein the treatment is FGF-21 treatment, GLP-1 treatment, treatment with an FGF-21- or GLP-1-containing therapeutic, cancer therapy (e.g., chemotherapy or radiation), bariatric surgery (e.g., gastric bypass), androgen or estrogen deprivation therapy, or treatment for obesity or Type 2 diabetes.
E203. The method of any one of E136, E138, E140, E157, and E158, wherein the diet-related bone loss is rickets.
E204. The method of any one of E131-E203, wherein the subject is at risk of bone fracture.
E205. The method of any one of E131-E204, wherein the method increases bone formation in the subject.
E206. The method of any one of E131-E205, wherein the method decreases bone resorption in the subject.
E207. The method of any one of E131-E206, wherein the method decreases bone loss in the subject.
E208. The method of any one of E131-E207, wherein the method increases osteoblast activity or osteoblastogenesis.
E209. The method of any one of E131-E208, wherein the method decreases osteoclast activity or decreases osteoclastogenesis.
E210. The method of any one of E131-E209, wherein the method decreases the risk or occurrence of bone fracture.
E211. The method of any one of E131-E210, wherein the method increases bone strength.
E212. The method of any one of E131-E211, wherein the bone is cortical bone.
E213. The method of any one of E131-E211, wherein the bone is trabecular bone.
E214. A method of decreasing or preventing fibrosis in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E215. A method of slowing or inhibiting the progression of fibrosis in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E216. A method of reducing the risk of developing fibrosis in a subject in need thereof by administering to the subject a therapeutically effective amount of the polypeptide of any one of E1-E80, the nucleic acid molecule of E1-E89, the nucleic acid molecule of E90, the vector of E91, the construct of E96 or E97, or the pharmaceutical composition of E94 or E95.
E217. A method of treating a subject having or at risk of developing fibrosis by administering to the subject a therapeutically effective amount of the polypeptide of any one of E1-E89, the nucleic acid molecule of E90, the vector of E91, the construct of E96 or E97, or the pharmaceutical composition of E94 or E95.
E218. A method of reversing fibrosis in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E219. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing fibrosis or a disease or condition involving fibrosis by administering to the subject a therapeutically effective amount of the polypeptide any one of E1-E89, the nucleic acid molecule of E90, the vector of E91, the construct of E96 or E97, or the pharmaceutical composition of E94 or E95.
E220. The method of any one of E214-E219, wherein the fibrosis or disease or condition involving fibrosis is chemotherapeutic drug-induced fibrosis, radiation-induced fibrosis, pulmonary fibrosis, hepatic fibrosis, renal fibrosis (e.g., fibrosis related to chronic kidney disease), corneal fibrosis, heart fibrosis, bone marrow fibrosis, myelofibrosis, mediastinal fibrosis, retroperitoneal fibrosis, arthrofibrosis, osteoarticular fibrosis, tissue fibrosis, a tumor stroma, a desmoplastic tumor, a surgical adhesion, a hypertrophic scar, or a keloid.
E221. The method of any one of E214-E219, wherein the fibrosis or disease or condition involving fibrosis is fibrosis associated with a wound, a burn, hepatitis B or C infection, fatty liver disease, Schistosoma infection, kidney disease (e.g., chronic kidney disease), heart disease, macular degeneration, Crohn's disease, retinal or vitreal retinopathy, systemic or local scleroderma, atherosclerosis, or restenosis.
E222. The method of any one of E214-E220, wherein, wherein the fibrosis results from chronic kidney disease.
E223. The method of any one of E214-E220, wherein the fibrosis is myelofibrosis.
E224. The method of E220, wherein the tissue fibrosis is fibrosis affecting a tissue selected from the group consisting of muscle tissue, skin epidermis, skin dermis, tendon, cartilage, pancreatic tissue, uterine tissue, neural tissue, testis, ovary, adrenal gland, artery, vein, bone marrow, colon, small intestine, large intestine, biliary tract, and gut.
E225. The method of any one of E214-E224, wherein the method improves the function of a fibrotic tissue or organ.
E226. The method of any one of E214-E225, wherein the method slows, inhibits, or reverses the development or progression of fibrosis.
E227. The method of any one of E214-E226, wherein the method reduces (e.g., reduces the frequency or severity of) one or more symptom of fibrosis.
E228. A method of increasing red blood cell levels (e.g., increasing hemoglobin levels, red blood cell count, and/or hematocrit) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E229. A method of increasing hemoglobin levels in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E230. A method of increasing red blood cell count in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E231. A method of increasing hematocrit in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E232. A method of promoting or increasing red blood cell production in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E233. A method of promoting or increasing maturation and/or differentiation of erythroid progenitors (e.g., early-stage or late (e.g., terminal) stage erythroid progenitors, e.g., the maturation and/or differentiation of early-stage erythroid progenitors, such as colony forming unit-erythroid cells (CFU-Es) and burst forming unit-erythroid cells (BFU-Es), into proerythroblasts, reticulocytes, or red blood cells) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E234. A method of promoting or increasing proerythroblasts (e.g., proerythroblast numbers or proerythroblast count) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E235. A method of promoting or increasing reticulocytes (e.g., reticulocyte numbers or reticulocyte count) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E236. A method of promoting or increasing the recruitment of early-stage progenitors into the erythroid lineage in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E237. A method of promoting or increasing late-stage erythroid precursor maturation (e.g., terminal maturation, such as the maturation of reticulocytes into red blood cells or the maturation of erythroblasts into reticulocytes and/or red blood cells) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E238. A method of reducing the accumulation of red blood cell progenitor cells in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E239. A method of increasing the number of early-stage erythroid precursors and/or progenitors (e.g., expanding the early-stage precursor and/or progenitor population) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E240. A method of promoting the progression of erythroid precursors and/or progenitors through erythropoiesis in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E241. The method of any one of E228-E240, wherein the subject has or is at risk of developing anemia or blood loss.
E242. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing a disease or condition involving low red blood cell levels (e.g., low hemoglobin levels, low red blood cell count, and/or low hematocrit) by administering to the subject a therapeutically effective amount of the polypeptide of any one of E1-E89, the nucleic acid molecule of E90, the vector of E91, the construct of E96 or E97, or the pharmaceutical composition of E94 or E95.
E243. The method of E242, wherein the disease or condition is anemia or blood loss.
E244. A method of treating a subject having or at risk of developing anemia by administering to the subject a therapeutically effective amount of the polypeptide of any one of E1-E89, the nucleic acid molecule of E90, the vector of E91, the construct of E96 or E97, or the pharmaceutical composition of E94 or E95.
E245. The method of E241, E243, or E244, wherein the anemia or blood loss is associated with cancer (e.g., a solid tumor, such as breast cancer, lung cancer, colon cancer; a tumor of the lymphatic system, such as chronic lymphocytic leukemia, non-Hodgkin's lymphoma, Hodgkin's lymphoma; or a tumor of the hematopoietic system, such as leukemia or multiple myeloma), cancer treatment (e.g., chemotherapy or radiation), myelofibrosis treatment (e.g., treatment with a JAK inhibitor, such as ruxolitinib, fedratinib, or pacritinib), renal disease or failure (e.g., chronic kidney disease or acute renal disease or failure), a myelodysplastic syndrome, thalassemia (e.g., α- or β-thalassemia), a nutritional deficit (e.g., vitamin deficiency, such as B-12 or folate deficiency), adverse reaction to medication, ineffective hematopoiesis, an inflammatory or autoimmune disease, splenomegaly, porphyria, vasculitis, hemolysis, a bone marrow defect, bone marrow transplantation, myelofibrosis, diabetes, liver disease (e.g., acute liver disease or chronic liver disease), bleeding (e.g., acute or chronic bleeding), infection, hemoglobinopathy, drug use, alcohol abuse, advanced age, Churg-Strauss syndrome, Felty syndrome, Pearson syndrome, dyskeratosis congenita, graft versus host disease, hematopoietic stem cell transplantation, osteomyelofibrosis, pancytopenia, pure red-cell aplasia, purpura Schoenlein-Henoch, Shwachman syndrome (e.g., Shwachman-Diamond syndrome), contraindication to transfusion, surgery, trauma, a wound, an ulcer, urinary tract bleeding, digestive tract bleeding, frequent blood donation, or heavy menstrual bleeding.
E246. The method of any one of E241 and E243-E245, wherein the anemia results from chronic kidney disease.
E247. The method of any one of E241 and E243-E245, wherein the anemia is associated with a myelodysplastic syndrome (e.g., the subject has a myelodysplastic syndrome).
E248. The method of any one of E241 and E243-E245, wherein the anemia is associated with myelofibrosis (e.g., the subject has myelofibrosis).
E249. The method of any one of E241 and E243-E245, wherein anemia is associated with ineffective hematopoiesis.
E250. The method of any one of E241 and E243-E249, wherein the anemia is aplastic anemia, iron deficiency anemia, vitamin deficiency anemia, anemia of chronic disease (also called anemia of inflammation), anemia associated with bone marrow disease, hemolytic anemia, sickle cell anemia, microcytic anemia, hypochromic anemia, sideroblastic anemia, congenital dyserythropoietic anemia, Diamond Blackfan anemia, Fanconi anemia, or refractory anemia with excess of blasts.
E251. The method of E250, wherein the sideroblastic anemia is acquired sideroblastic anemia or congenital sideroblastic anemia.
E252. The method of E251, wherein the sideroblastic anemia is congenital sideroblastic anemia.
E253. The method of E252, wherein the congenital sideroblastic anemia is associated with a mutation in ALAS2, SLC25A38, FECH, GLRX5, HSPA9, HSCB, SLC25A38, or ABCB7.
E254. The method of E252, wherein the congenital sideroblastic anemia is associated with a mutation in PUS1, YARS2, LARS2, TRNT1, MT-ATP6, NDUFB11, or SLC19A2, or with an mtDNA mutation.
E255. The method of any one of E228-E254, wherein the method increases red blood cell production, red blood cell count, hemoglobin levels, hematocrit, erythrocyte progenitor differentiation and/or maturation (e.g., of early and/or terminal stage erythroid progenitors), late-stage erythroid precursor maturation, recruitment of early-stage progenitors into the erythroid lineage, proerythroblast numbers, early-stage erythroid precursor and/or progenitor numbers (e.g., increases the early-stage precursor and/or progenitor populations), the progression of erythroid precursors and/or progenitor through erythropoiesis, and/or reticulocyte numbers.
E256. The method of any one of E228-E255, wherein the method reduces the accumulation of red blood cell progenitor cells.
E257. The method of any one of E228-E256, wherein the subject is identified as having anemia prior to administration of the polypeptide of any one of E1-E89, the nucleic acid molecule of E90, the vector of E91, the construct of E96 or E97, or the pharmaceutical composition of E94 or E95.
E258. The of any one of E228-E256, wherein the method further comprises identifying the subject as having anemia prior to administration of the polypeptide of any one of E1-E89, the nucleic acid molecule of E90, the vector of E91, the construct of E96 or E97, or the pharmaceutical composition of E94 or E95.
E259. The method of any one of E228-E256, wherein the method further comprises evaluating red blood cell, hemoglobin, hematocrit, and/or reticulocyte levels after administration of the polypeptide of any one of E1-E89, the nucleic acid molecule of E90, the vector of E91, the construct of E96 or E97, or the pharmaceutical composition of E94 or E95.
E260. A method of increasing platelet levels (e.g., increasing platelet count) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E261. A method of increasing platelet count in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E262. A method of promoting or increasing platelet production in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E263. A method of promoting or increasing megakaryocyte differentiation and/or maturation (e.g., to produce platelets) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E264. A method of reducing the accumulation of platelet progenitor cells in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E265. The method of any one of E260-E264, wherein the subject has or is at risk of developing thrombocytopenia.
E266. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing a disease or condition involving low platelet levels, wherein the method includes administering to the subject a therapeutically effective amount of the polypeptide of any one of E1-E89, the nucleic acid molecule of E90, the vector of E91, the construct of E96 or E97, or the pharmaceutical composition of E94 or E95.
E267. The method of E266, wherein the disease or condition is thrombocytopenia.
E268. A method of treating a subject having or at risk of developing thrombocytopenia by administering to the subject a therapeutically effective amount of the polypeptide of any one of E1-E89, the nucleic acid molecule of E90, the vector of E91, the construct of E96 or E97, or the pharmaceutical composition of E94 or E95.
E269. A method of promoting platelet production by contacting a megakaryocyte with the polypeptide of any one of E1-E89, the nucleic acid molecule of E90, the vector of E91, or the construct of E96 or E97 in an amount effective to promote platelet production.
E270. The method of E269, wherein the contacting is in vitro.
E271. A method of treating a subject having or at risk of developing thrombocytopenia by administering to the subject a platelet produced by the method of E269 or E270.
E272. The method of any one of E265, E267, E268, and E271, wherein the thrombocytopenia is associated with a bone marrow defect, a myelodysplastic syndrome, bone marrow transplantation, myelofibrosis, myelofibrosis treatment (e.g., treatment with a JAK inhibitor, such as with ruxolitinib, fedratinib, or pacritinib), ineffective hematopoiesis, Gaucher disease, aplastic anemia, Fanconi anemia, Diamond Blackfan anemia, Shwachman Diamond syndrome, heavy alcohol consumption, cirrhosis of the liver, cancer (e.g., leukemia or lymphoma), an autoimmune disease, a viral infection, a bacterial infection, an enlarged spleen, a vitamin deficiency, cancer treatment, thrombotic thrombocytopenia purpura, idiopathic thrombocytopenia purpura, disseminated intravascular coagulation, hemolytic uremic syndrome, paroxysmal nocturnal hemoglobinuria, a reduction of platelets caused by medication (medication-induced thrombocytopenia, e.g., thrombocytopenia caused by treatment with heparin, quinine, a sulfa-containing antibiotic, such as vancomycin, rifampin, or trimethoprim, or an anticonvulsant, such as phenytoin), hematopoietic stem cell transplantation, acquired amegakaryocytic thrombocytopenia, Pearson syndrome, dyskeratosis congenita, contraindication to transfusion, or a dilution of platelets caused by a blood transfusion.
E273. The method of any one of E265, E267, E268, E271, and E272, wherein the thrombocytopenia is associated with a myelodysplastic syndrome (e.g., the subject has a myelodysplastic syndrome).
E274. The method of any one of E265, E267, E268, E271, and E272, wherein the thrombocytopenia is associated with myelofibrosis (e.g., the subject has myelofibrosis).
E275. The method of any one of E265, E267, E268, E271, and E272, wherein the thrombocytopenia is associated with a bone marrow defect.
E276. The method of any one of E265, E267, E268, E271, and E272, wherein the thrombocytopenia is associated with bone marrow transplantation.
E277. The method of any one of E265, E267, E268, E271, and E272, wherein the thrombocytopenia is associated with cancer.
E278. The method of any one of E265, E267, E268, E271, and E272, wherein the thrombocytopenia is associated with cancer treatment (e.g., chemotherapy or radiation).
E279. The method of any one of E265, E267, E268, E271, and E272, wherein the thrombocytopenia is associated with hematopoietic stem cell transplantation.
E280. The method of any one of E265, E267, E268, E271, and E272, wherein the thrombocytopenia is associated with an autoimmune disease.
E281. The method of any one of E265, E267, E268, E271, and E272, wherein the thrombocytopenia is associated with contraindication to transfusion.
E282. The method of any one of E265, E267, E268, E271, and E272, wherein the thrombocytopenia is associated with myelofibrosis treatment.
E283. The method of any one of E265, E267, E268, E271, and E272, wherein the thrombocytopenia is associated with ineffective hematopoiesis.
E284. The method of any one of E265, E267, E268, E271, and E272, wherein the thrombocytopenia is associated with thrombotic thrombocytopenia purpura.
E285. The method of E265, E267, E268, and E271, wherein the thrombocytopenia is familial thrombocytopenia.
E286. The method of E285, wherein the familial thrombocytopenia is May-Hegglin anomaly, Sebastian syndrome, Fechtner syndrome, Epstein's syndrome, Wiskott-Aldrich syndrome, congenital amegakaryocytic thrombocytopenia, platelet storage pool deficiency, Hermansky-Pudlak syndrome, Bernard-Soulier syndrome, Von Willebrand Disease Type 2B, ANKRD26-related thrombocytopenia, thrombocytopenia absent radius syndrome, familial platelet disorder with associated myeloid malignancy (FPD/AML), thrombocytopenia associated with a mutation in Filamin-A, or thrombocytopenia associated with a mutation in GATA-1.
E287. The method of E265, E267, E268, and E271, wherein the thrombocytopenia is immune thrombocytopenia.
E288. The method of any one of E260-E287, wherein the method increases platelet count, platelet production and/or megakaryocyte differentiation and/or maturation.
E289. The method of any one of E260-E288, wherein the method reduces the accumulation of platelet progenitor cells.
E290. The method of any one of E260-E289, wherein the method improves blood clotting, reduces bleeding events (e.g., reduces the incidence of bleeding events), and/or reduces bleeding in the skin of the subject.
E291. The method of any one of E260-E269 and E271-E290, wherein the subject is identified as having thrombocytopenia prior to administration of the polypeptide of any one of E1-E89, the nucleic acid molecule of E90, the vector of E91, the construct of E96 or E97, the pharmaceutical composition of E94 or E95, or the platelets produced by the method of E269 or E270.
E292. The method of any one of E260-E269 and E271-E290, wherein the method further comprises identifying the subject as having thrombocytopenia prior to administration of the polypeptide of any one of E1-E89, the nucleic acid molecule of E90, the vector of E91, the construct of E96 or E97, the pharmaceutical composition of E94 or E95, or the platelets produced by the method of E269 or E270.
E293. The method of any one of E260-E269 and E271-E292, wherein the method further comprises evaluating platelet levels after administration of the polypeptide of any one of E1-E89, the nucleic acid molecule of E90, the vector of E91, the construct of E96 or E97, the pharmaceutical composition of E94 or E95, or the platelets produced by the method of E269 or E270.
E294. A method of increasing neutrophil levels (e.g., increasing neutrophil count) in a subject in need thereof by administering to the subject a therapeutically effective amount of the polypeptide of any one E1-E89, the nucleic acid molecule of E90, the vector of E91, the construct of E96 or E97, or the pharmaceutical composition of E94 or E95.
E295. A method of increasing neutrophil count in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E296. A method of promoting or increasing neutrophil production in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E297. A method of promoting or increasing the differentiation and/or maturation of progenitor cells (e.g., myeloid progenitors, myeloblasts, and/or myelocytes) into neutrophils in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E298. The method of any one of E294-E297, wherein the subject has or is at risk of developing neutropenia.
E299. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing a disease or condition involving low neutrophil levels, wherein method includes administering to the subject a therapeutically effective amount of the polypeptide of any one of E1-E89, the nucleic acid molecule of E90, the vector of E91, the construct of E96 or E97, or the pharmaceutical composition of E94 or E95.
E300. The method of E299, wherein the disease or condition is neutropenia.
E301. A method of treating a subject having or at risk of developing neutropenia by administering to the subject a therapeutically effective amount of the polypeptide of any one of E1-E89, the nucleic acid molecule of E90, the vector of E91, the construct of E96 or E97, or the pharmaceutical composition of E94 or E95.
E302. The method of E298, E300, or E301, wherein the neutropenia is associated with a bone marrow defect, a myelodysplastic syndrome, bone marrow transplantation, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer (e.g., leukemia), a vitamin deficiency, an enlarged spleen, an autoimmune disease, a viral infection, a bacterial infection, cancer treatment, a reduction in neutrophils caused by medication (e.g., medication used to treat overactive thyroid, such as methimazole and propylthiouracil; an antibiotic, such as vancomycin, penicillin G, trimethoprim, and oxacillin; an antiviral drug, such as ganciclovir and valganciclovir; an anti-inflammatory medication for ulcerative colitis or rheumatoid arthritis, such as sulfasalazine; a drug used to treat irregular heart rhythms, such as quinidine and procainamide; an anticonvulsant, such as phenytoin and valproate; an antipsychotic, such as clozapine; or levamisole), inflammation, hematopoietic stem cell transplantation, or contraindication to transfusion.
E303. The method of any one of E298 and E300-E302, wherein the neutropenia is associated with a myelodysplastic syndrome (e.g., the subject has a myelodysplastic syndrome).
E304. The method of any one of E298 and E300-E302, wherein the neutropenia is associated with myelofibrosis (e.g., the subject has myelofibrosis).
E305. The method of any one of E298 and E300-E302, wherein the neutropenia is associated with a bone marrow defect.
E306. The method of any one of E298 and E300-E302, wherein the neutropenia is associated with bone marrow transplantation.
E307. The method of any one of E298 and E300-E302, wherein the neutropenia is associated with cancer.
E308. The method of any one of E298 and E300-E302, wherein the neutropenia is associated with cancer treatment (e.g., chemotherapy or radiation).
E309. The method of any one of E298 and E300-E302, wherein the neutropenia is associated with hematopoietic stem cell transplantation.
E310. The method of any one of E298 and E300-E302, wherein the neutropenia is associated with inflammation.
E311. The method of any one of E298 and E300-E302, wherein the neutropenia is associated with contraindication to transfusion.
E312. The method of any one of E298 and E300-E302, wherein the neutropenia is associated with ineffective hematopoiesis.
E313. The method of E298, E300, or E301, wherein the neutropenia is chronic idiopathic neutropenia.
E314. The method of E298, E300, or E301, wherein the neutropenia is familial neutropenia.
E315. The method of E314, wherein the familial neutropenia is cyclic neutropenia, chronic benign neutropenia, or severe congenital neutropenia (e.g., neutropenia associated with mutations in the genes ELANE (associated with SCN1), HAX1 (associated with SCN3), G6PC3 (associated with SCN4), GFI1 (associated with SCN2), CSF3R, WAS (associated with X-linked neutropenia/X-linked SCN), CXCR4, VPS45A (associated with SCN5), or JAGN1).
E316. The method of any one of E294-E315, wherein the method increases neutrophil count, neutrophil production, and/or the differentiation and/or maturation of progenitor cells into neutrophils.
E317. The method of any one of E294-E316, wherein the method reduces the subject's susceptibility to infection.
E318. The method of any one of E294-E317, wherein the subject is identified as having neutropenia prior to administration of the polypeptide of any one of E1-E89, the nucleic acid molecule of E90, the vector of E91, the construct of E96 or E97, or the pharmaceutical composition of E94 or E95.
E319. The method of any one of E294-E317, wherein the method further comprises identifying the subject as having neutropenia prior to administration of the polypeptide of any one of E1-E89, the nucleic acid molecule of E90, the vector of E91, the construct of E96 or E97, or the pharmaceutical composition of E94 or E95.
E320. The method of any one of E294-E319, wherein the method further comprises evaluating neutrophil levels after administration of the polypeptide of any one of E1-E89, the nucleic acid molecule of E90, the vector of E91, the construct of E96 or E97, or the pharmaceutical composition of E94 or E95.
E321. A method of treating a subject having or at risk of developing a myelodysplastic syndrome by administering to the subject a therapeutically effective amount of the polypeptide of any one of E1-E89, the nucleic acid molecule of E90, the vector of E91, the construct of E96 or E97, or the pharmaceutical composition of E94 or E95.
E322. A method of increasing red blood cell count, increasing hemoglobin levels, increasing hematocrit, increasing red blood cell production, increasing or inducing erythroid progenitor cell maturation and/or differentiation, increasing or inducing late-stage erythroid precursor maturation, promoting or increasing recruitment of early-stage progenitors into the erythroid lineage, increasing proerythroblasts, increasing early-stage erythroid precursor and/or progenitor numbers (e.g., increasing the early-stage precursor and/or progenitor population), promoting the progression of erythroid precursors and/or progenitors through erythropoiesis, increasing reticulocytes, increasing platelet count, increasing platelet production, increasing or inducing megakaryocyte differentiation and/or maturation, reducing the accumulation of platelet progenitor cells, increasing neutrophil count, increasing neutrophil production, increasing or inducing the differentiation and/or maturation of progenitor cells into neutrophils, improving blood clotting, reducing bleeding events, reducing bleeding in the skin, and/or reducing susceptibility to infection in a subject having or at risk of developing a myelodysplastic syndrome by administering to the subject a therapeutically effective amount of the polypeptide of any one of E1-E89, the nucleic acid molecule of E90, the vector of E91, the construct of E96 or E97, or the pharmaceutical composition of E94 or E95.
E323. The method of any one of E245, E247, E272, E273, E302, E303, E321, and E322, wherein the myelodysplastic syndrome is myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (myelodysplastic syndrome with isolated del (5q)), myelodysplastic syndrome with excess blasts (e.g., myelodysplastic syndrome with excess blasts-type 1 (MDS-EB-1) or myelodysplastic syndrome with excess blasts-type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T).
E324. The method of any one of E245, E247, E272, E273, E302, E303, and E321-E323, wherein the myelodysplastic syndrome is MDS-SLD.
E325. The method of any one of E245, E247, E272, E273, E302, E303, and E321-E323, wherein the myelodysplastic syndrome is MDS-MLD.
E326. The method of any one of E245, E247, E272, E273, E302, E303, and E321-E323, wherein the myelodysplastic syndrome is MDS-RS-SLD.
E327. The method of any one of E245, E247, E272, E273, E302, E303, and E321-E323, wherein the myelodysplastic syndrome is MDS-RS-MLD.
E328. The method of any one of E245, E247, E272, E273, E302, E303, and E321-E323, wherein the myelodysplastic syndrome is myelodysplastic syndrome with isolated del (5q).
E329. The method of any one of E245, E247, E272, E273, E302, E303, and E321-E323, wherein the myelodysplastic syndrome is MDS-EB-1.
E330. The method of any one of E245, E247, E272, E273, E302, E303, and E321-E323, wherein the myelodysplastic syndrome is MDS-EB-2.
E331. The method of any one of E245, E247, E272, E273, E302, E303, and E321-E323, wherein the myelodysplastic syndrome is MDS-U.
E332. The method of any one of E245, E247, E272, E273, E302, E303, and E321-E323, wherein the myelodysplastic syndrome is MDS/MPN-RS-T.
E333. The method of any one of E245, E247, E272, E273, E302, E303, and E321-E332, wherein the myelodysplastic syndrome is a ring sideroblast positive myelodysplastic syndrome (RS positive MDS, e.g., the subject has ring sideroblasts).
E334. The method of E333, wherein the RS-positive myelodysplastic syndrome is associated with a splicing factor mutation.
E335. The method of E334, wherein the splicing factor mutation is a mutation in Splicing Factor 3b Subunit 1 (SF3B1).
E336. The method of any one of E245, E247, E272, E273, E302, E303, E321-E325, and E328-E332, wherein the myelodysplastic syndrome is a non-ring sideroblast myelodysplastic syndrome (non-RS, e.g., the subject lacks ring sideroblasts).
E337. The method of any one of E245, E247, E272, E273, E302, E303, and E321-E336, wherein the myelodysplastic syndrome is a very low, low, or intermediate risk myelodysplastic syndrome (e.g., as determined by the Revised International Prognostic Scoring System).
E338. The method of E337, wherein the myelodysplastic syndrome is a very low risk myelodysplastic syndrome (e.g., as determined by the Revised International Prognostic Scoring System).
E339. The method of E337, wherein the myelodysplastic syndrome is a low risk myelodysplastic syndrome (e.g., as determined by the Revised International Prognostic Scoring System).
E340. The method of E337, wherein the myelodysplastic syndrome is an intermediate risk myelodysplastic syndrome (e.g., as determined by the Revised International Prognostic Scoring System).
E341. The method of any one of E245, E247, E272, E273, E302, E303, and E321-E340, wherein the myelodysplastic syndrome is associated with a defect in terminal maturation.
E342. The method of any one of E245, E247, E272, E273, E302, E303, and E321-E341, wherein the myelodysplastic syndrome is associated with a defect in early-stage hematopoiesis (e.g., commitment or differentiation of progenitor cells).
E343. The method of any one of E245, E247, E272, E273, E302, E303, and E321-E342, wherein the myelodysplastic syndrome is associated with elevated endogenous erythropoietin levels.
E344. The method of any one of E245, E247, E272, E273, E302, E303, and E321-E343, wherein the myelodysplastic syndrome is associated with hypocellular bone marrow (e.g., the subject has hypocellular bone marrow).
E345. A method of treating a subject having or at risk of developing myelofibrosis by administering to the subject a therapeutically effective amount of the polypeptide of any one of E1-E89, the nucleic acid molecule of E90, the vector of E91, the construct of E96 or E97, or the pharmaceutical composition of E94 or E95.
E346. A method of increasing red blood cell count, increasing hemoglobin levels, increasing hematocrit, increasing red blood cell production, increasing or inducing erythroid progenitor cell maturation and/or differentiation, increasing or inducing late-stage erythroid precursor maturation, promoting or increasing recruitment of early-stage progenitors into the erythroid lineage, increasing proerythroblasts, increasing early-stage erythroid precursor and/or progenitor numbers (e.g., increasing the early-stage precursor and/or progenitor population), promoting the progression of erythroid precursors and/or progenitors through erythropoiesis, increasing reticulocytes, increasing platelet count, increasing platelet count, increasing platelet production, increasing or inducing megakaryocyte differentiation and/or maturation, reducing the accumulation of platelet progenitor cells, increasing neutrophil count, increasing neutrophil production, increasing or inducing the differentiation and/or maturation of progenitor cells into neutrophils, improving blood clotting, reducing bleeding events, reducing bleeding in the skin, and/or reducing susceptibility to infection in a subject having or at risk of developing myelofibrosis by administering to the subject a therapeutically effective amount of the polypeptide of any one E1-E89, the nucleic acid molecule of E90, the vector of E91, the construct of E96 or E97, or the pharmaceutical composition of E94 or E95.
E347. The method of any one of E228-E346, wherein the subject does not respond well to treatment with erythropoietin (EPO), is susceptible to the adverse effects of EPO, or does not respond well to treatment with an erythroid maturation agent.
E348. The method of any one of E228-E347, wherein the subject has previously been treated with an erythropoiesis stimulating agent (ESA).
E349. The method of any one of E228-E347, wherein the subject has not previously been treated with an erythropoiesis stimulating agent (ESA).
E350. The method of any one of E228-E349, wherein the subject has a low transfusion burden.
E351. The method of E350, wherein the subject has received 1-3 units of RBCs (1-3 RBC transfusions) within eight weeks prior to starting treatment with the polypeptide of any one of E1-E89, the nucleic acid molecule of E90, the vector of E91, the construct of E96 or E97, or the pharmaceutical composition of E94 or E95.
E352. The method of E350, wherein the subject has received 0 units of RBCs (0 RBC transfusions) within eight weeks prior to starting treatment with the polypeptide of any one of E1-E89, the nucleic acid molecule of E90, the vector of E91, the construct of E96 or E97, or the pharmaceutical composition of E94 or E95.
E353. The method of any one of E228-E349, wherein the subject has a high transfusion burden.
E354. The method of any one of E228-E353, wherein the method reduces the subject's need for a blood transfusion (e.g., reduces transfusion burden).
E355. A method of preventing (e.g., preventing the development of) pulmonary hypertension (PH) in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E356. A method of reducing the risk of developing PH in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E357. A method of slowing or inhibiting the progression of PH in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E358. A method of treating a subject having or at risk of developing PH by administering to the subject a therapeutically effective amount of the polypeptide of any one of E1-E89, the nucleic acid molecule of E90, the vector of E91, the construct of E96 or E97, or the pharmaceutical composition of E94 or E95.
E359. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing PH by administering to the subject a therapeutically effective amount of the polypeptide of any one of E1-E89, the nucleic acid molecule of E90, the vector of E91, the construct of E96 or E97, or the pharmaceutical composition of E94 or E95.
E360. A method of reducing vascular remodeling in a subject having or at risk of developing PH by administering to the subject a therapeutically effective amount of the polypeptide of any one of E1-E89, the nucleic acid molecule of E90, the vector of E91, the construct of E96 or E97, or the pharmaceutical composition of E94 or E95.
E361. A method of reducing right ventricular hypertrophy in a subject having or at risk of developing PH by administering to the subject a therapeutically effective amount of the polypeptide of any one of E1-E89, the nucleic acid molecule of E90, the vector of E91, the construct of E96 or E97, or the pharmaceutical composition of E94 or E95.
E362. A method of reducing pulmonary vascular resistance in a subject having or at risk of developing PH by administering to the subject a therapeutically effective amount of the polypeptide of any one of E1-E89, the nucleic acid molecule of E90, the vector of E91, the construct of E96 or E97, or the pharmaceutical composition of E94 or E95.
E363. The method of any one of E355-E362, wherein the PH is pulmonary arterial hypertension (PAH).
E364. The method of E363, wherein the PAH is idiopathic PAH.
E365. The method of E363, wherein the PAH is heritable PAH.
E366. The method of E363, wherein the PAH is associated with HIV infection, schistosomiasis, cirrhosis of the liver, a congenital heart abnormality, portal hypertension, pulmonary veno-occlusive disease, pulmonary capillary hemangiomatosis, a connective tissue disorder, an autoimmune disorder (e.g., scleroderma or lupus), or drug use or abuse (e.g., use of cocaine or methamphetamine).
E367. The method of any one of E355-E362, wherein the PH is venous PH.
E368. The method of E367, wherein the venous PH is associated with left ventricular systolic dysfunction, left ventricular diastolic dysfunction, valvular heart disease, congenital cardiomyopathy, or congenital or acquired pulmonary venous stenosis.
E369. The method of any one of E355-E362, wherein the PH is hypoxic PH.
E370. The method of E369, wherein the hypoxic PH is associated with chronic obstructive pulmonary disease (e.g., emphysema), interstitial lung disease, sleep-disordered breathing (e.g., sleep apnea), a lung disease (e.g., pulmonary fibrosis), an alveolar hypoventilation disorder, chronic exposure to high altitude, or a developmental abnormality.
E371. The method of any one of E355-E362, wherein the PH is thromboembolic PH.
E372. The method of E371, wherein the thromboembolic PH is associated with chronic thromboembolic pulmonary hypertension, pulmonary emboli, angiosarcoma, arteritis, congenital pulmonary artery stenosis, or parasitic infection.
E373. The method of any one of E355-E362, wherein the PH is miscellaneous PH.
E374. The method of any one of E373, wherein the miscellaneous PH is associated with a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or thyroid diseases), pulmonary tumoral thrombotic microangiopathy, fibrosing mediastinitis, chronic kidney failure, or segmental pulmonary hypertension.
E375. The method of any one of E355-E374, wherein the method reduces the frequency or severity of one or more symptoms of PH (e.g., reduces the severity or frequency of one or more of shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting).
E376. The method of any one of E355-E375, wherein the method reduces pulmonary vascular remodeling.
E377. The method of any one of E355-E376, wherein the method reduces vascular remodeling in the heart.
E378. The method of any one of E355-E377, wherein the method reduces right ventricular hypertrophy. E379. The method of any one of E355-E378, wherein the method reduces pulmonary vascular resistance (e.g., reduces pulmonary vascular resistance compared to measurements taken prior to treatment).
E380. The method of any one of E355-E379, wherein the method improves performance in the 6-minute walk test (e.g., improves performance compared to measurements taken prior to treatment).
E381. The method of any one of E355-E380, wherein the method reduces bone loss.
E382. The method of any one of E355-E381, wherein the method reduces pulmonary arterial muscularization or pulmonary arterial wall thickening.
E383. The method of any one of E355-E382, wherein the method reduces right ventricular compensation.
E384. A method of reducing body fat in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E385. A method of reducing body weight in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E386. A method of reducing blood glucose in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E387. A method of increasing insulin sensitivity in a subject in need thereof, by administering to the subject a therapeutically effective amount of the polypeptide of any one of E1-E89, the nucleic acid molecule of E90, the vector of E91, the construct of E96 or E97, or the pharmaceutical composition of E94 or E95.
E388. The method of any one of E384-E387, wherein the subject has or is at risk of developing a metabolic disease.
E389. A method of affecting myostatin, activin A, activin B, and/or BMP9 signaling (e.g., reducing or inhibiting the binding of myostatin, activin A, activin B, and/or BMP9 to their receptors) in a subject having or at risk of developing a metabolic disease by administering to the subject a therapeutically effective amount of the polypeptide of any one of E1-E89, the nucleic acid molecule of E90, the vector of E91, the construct of E96 or E97, or the pharmaceutical composition of E94 or E95.
E390. A method of treating and/or preventing a metabolic disease in a subject by administering to the subject a therapeutically effective amount of the polypeptide of any one of E1-E89, the nucleic acid molecule of E90, the vector of E91, the construct of E96 or E97, or the pharmaceutical composition of E94 or E95.
E391. The method of any one of E388-E390, wherein the metabolic disease is age-related metabolic disease.
E392. The method of any one of E388-E390, wherein the metabolic disease is treatment-related metabolic disease.
E393. The method of E392, wherein the treatment is treatment with a glucocorticoid (e.g., a corticosteroid, such as prednisone), a selective serotonin reuptake inhibitors (SSRI, e.g., paroxetine, mirtazapine, fluoxetine, escitalopram, or sertraline), a serotonin-norepinephrine reuptake inhibitors (SNRI), a tricyclic antidepressant (e.g., amitriptyline), a mood stabilizer (e.g., valproic acid or lithium), an antipsychotic (e.g., olanzapine, chlorpromazine, or clozapine), or a diabetes medication (e.g., insulin, chlorpropamide).
E394. The method of any one of E388-E393, wherein the metabolic disease is selected from the group including obesity, Type 1 diabetes, and Type 2 diabetes.
E395. The method of E394, wherein the metabolic disease is obesity.
E396. The method of E394, wherein the metabolic disease is Type 1 diabetes.
E397. The method of E394, wherein the metabolic disease is Type 2 diabetes.
E398. The method of any one of E384-E397, wherein the method reduces body weight and/or percentage of body weight gain of said subject.
E399. The method of any one of E384-E398, wherein the method reduces amount of body fat and/or percentage of body fat of said subject.
E400. The method of any one of E384-E399, wherein the method does not affect the appetite for food intake of said subject.
E401. The method of any one of E384-E400, wherein the method reduces adiposity of said subject.
E402. The method of any one of E384-E401, wherein the method reduces the weights of epididymal and perirenal fat pads of said subject.
E403. The method of any one of E384-E402, wherein, the method reduces the amount of subcutaneous, visceral, and/or hepatic fat of said subject.
E404. The method of any one of E384-E403, wherein the method lowers the level of fasting insulin of said subject.
E405. The method of any one of E384-E404, wherein the method lowers the level of blood glucose of said subject.
E406. The method of any one of E384-E405, wherein the method increases insulin sensitivity of said subject.
E407. The method of any one of E384-E406, wherein the method increases the rate of glucose clearance of said subject.
E408. The method of any one of E384-E407, wherein the method improves the serum lipid profile of said subject.
E409. The method of any one of E384-E408, wherein the method delays, reduces, or eliminates the need for insulin treatment.
E410. The method of any one of E384-E409, wherein the method does not reduce lean mass.
E411. A method of treating a subject having or at risk of developing a disease or condition that can be treated with erythropoietin or an erythropoiesis-stimulating agent by administering to the subject a therapeutically effective amount of the polypeptide of any one of E1-E89, the nucleic acid molecule of E90, the vector of E91, the construct of E96 or E97, or the pharmaceutical composition of E94 or E95.
E412. A method of affecting myostatin, activin A, activin B, and/or BMP9 signaling (e.g., reducing or inhibiting the binding of myostatin, activin A, activin B, and/or BMP9 to their endogenous receptors) in a subject having or at risk of developing a disease or condition that can be treated with erythropoietin or an erythropoiesis-stimulating agent by administering to the subject a therapeutically effective amount of the polypeptide of any one of E1-E89, the nucleic acid molecule of E90, the vector of E91, the construct of E96 or E97, or the pharmaceutical composition of E94 or E95.
E413. The method of E411 or E412, in which the disease or condition that can be treated with erythropoietin or an erythropoiesis-stimulating agent is anemia due to dialysis or anemia of prematurity.
E414. The method of E411 or E412, in which the disease or condition that can be treated with erythropoietin or an erythropoiesis-stimulating agent is end-stage renal disease, renal insufficiency, polycythemia, hemochromatosis, a disease or condition associated with dysfunction of endothelial progenitor cells, a disease or condition having an autoimmune or inflammatory component, a neurological disorder or inflammatory brain disease, gastrointestinal dysmotility, a disease of the endocrine system, a disease of the reproductive system, aging, pregnancy, a menstrual disorder, ischemia or an ischemic disorder or condition, hypoxia or a hypoxic disorder or condition, an ulcer, a burn, a wound (e.g., a chronic wound), ischemia-reperfusion injury, asthma, hypertension, a viral disease or infection, a systemic microbial infection, a gastrointestinal disease, arterial sclerosis, cancer, psychosis, a genetic disease, an inflammatory disease, graft-versus-host disease, cardiovascular disease, an allergy, or arthritis.
E415. A method of increasing erythropoietin levels in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E416. A method of increasing erythropoietin receptor levels in a subject in need thereof by administering to the subject a therapeutically effective amount of the polypeptide of any one of E1-E89, the nucleic acid molecule of E90, the vector of E91, the construct of E96 or E97, or the pharmaceutical composition of E94 or E95.
E417. The method of E415 or E416, in which the subject has or is at risk of developing anemia due to dialysis or anemia of prematurity.
E418. The method of E415 or E416, in which the subject has or is at risk of developing of end-stage renal disease, renal insufficiency, polycythemia, hemochromatosis, a disease or condition associated with dysfunction of endothelial progenitor cells, a disease or condition having an autoimmune or inflammatory component, a neurological disorder or inflammatory brain disease, gastrointestinal dysmotility, a disease of the endocrine system, a disease of the reproductive system, aging, pregnancy, a menstrual disorder, ischemia or an ischemic disorder or condition, hypoxia or a hypoxic disorder or condition, an ulcer, a burn, a wound (e.g., a chronic wound), ischemia-reperfusion injury, asthma, hypertension, a viral disease or infection, a systemic microbial infection, a gastrointestinal disease, arterial sclerosis, cancer, psychosis, a genetic disease, an inflammatory disease, graft-versus-host disease, cardiovascular disease, an allergy, or arthritis.
E419. The method of E414 or E418, in which the in which the disease or condition that can be treated with erythropoietin or an erythropoiesis-stimulating agent is ischemia or in which the subject has or is at risk of developing ischemia.
E420. The method of E419, in which the ischemia is central nervous system ischemia, liver ischemia, renal ischemia, or cardiac ischemia.
E421. The method of E414 or E418, in which the disease or condition that can be treated with erythropoietin or an erythropoiesis-stimulating agent is an ischemic disorder or condition or in which the subject has or is at risk of developing an ischemic disorder or condition.
E422. The method of E421, in which the ischemic disorder or condition is occlusive arterial disease, chronic venous insufficiency, circulatory shock (e.g., hemorrhagic, septic, or cardiogenic shock), pulmonary embolism, myocardial infarction, ischemic stroke, acute respiratory failure, chronic heart failure, atherosclerosis, cardiac cirrhosis, macular degeneration, sleep apnea, Raynaud's disease, systemic sclerosis, nonbacterial thrombotic endocarditis, a transient ischemic attack, or ischemia resulting from general anesthesia.
E423. The method of E414 or E418, in which the disease or condition that can be treated with erythropoietin or an erythropoiesis-stimulating agent is a hypoxic disorder or condition or in which the subject has or is at risk of developing a hypoxic disorder or condition.
E424. The method of E423, in which the hypoxic disorder or condition is a pulmonary disorder (e.g., chronic obstructive pulmonary disease), perinatal hypoxia, severe pneumonia, pulmonary edema, hyaline membrane disease, liver disease, renal disease, cancer, or altitude sickness.
E425. The method of E414 or E418, in which the disease or condition that can be treated with erythropoietin or an erythropoiesis-stimulating agent is a viral disease or infection or in which the subject has or is at risk of developing a viral disease or infection.
E426. The method of E425, in which the viral disease or infection is a Hepatitis C virus infection or an HIV infection.
E427. The method of E414 or E418, in which the disease or condition that can be treated with erythropoietin or an erythropoiesis-stimulating agent is a disease or condition associated with dysfunction of endothelial progenitor cells or in which the subject has or is at risk of developing a disease or condition associated with dysfunction of endothelial progenitor cells.
E428. The method of E427, in which the disease or condition associated with dysfunction of endothelial progenitor cells is heart failure, angina pectoris, endotheliosis, reticuloendotheliosis, age-related cardiovascular disorder, coronary heart disease, atherosclerosis, myocardial ischemia, hypercholesterolemia, an ischemic disorder of the extremities, Raynaud's disease, preeclampsia, pregnancy induced hypertension, an endothelium-mediated chronic inflammatory disorder (e.g., inflammation of the vessels), wound healing, chronic renal failure (chronic kidney disease), or acute renal failure (acute kidney failure).
E429. The method of E428, in which the disease or condition associated with dysfunction of endothelial progenitor cells is chronic renal failure (chronic kidney disease).
E430. The method of E414 or E418, in which the disease or condition that can be treated with erythropoietin or an erythropoiesis-stimulating agent is an autoimmune or inflammatory disease or condition or in which the subject has or is at risk of developing an autoimmune or inflammatory disease or condition.
E431. The method of E430, in which the autoimmune or inflammatory disease or condition is acute cerebrovascular injury, acute brain injury, acute cardiovascular injury, arthritis, an autoimmune disease, a stroke, a neurological injury, or immune-mediated inflammation.
E432. The method of E414 or E418, in which the disease or condition that can be treated with erythropoietin or an erythropoiesis-stimulating agent is a neurological disorder or inflammatory brain disease or in which the subject has or is at risk of developing a neurological disorder or inflammatory brain disease.
E433. The method of E432, in which the neurological disorder or inflammatory brain disease is a demyelinating disease, epilepsy, spinal cord injury (e.g., an acute spinal cord injury), a complication following traumatic brain injury (e.g., to treat a symptom of the traumatic brain injury, such as hypotension, hypoxemia, brain swelling, headache, neck pain, difficulty remembering, difficulty concentrating, difficulty making decisions, fatigue, a mood change, nausea, photophobia, blurred vision, ear ringing, a loss of sense of taste, and a loss of sense of smell, seizures, coma, muscle weakness, paralysis, or a progressive decline in neurologic function), a chronic inflammatory brain disease, or a neurological disorder associated with a surgery (e.g., thoracoabdominal aortic surgery).
E434. The method of E433, in which the chronic inflammatory brain disease is a neurodegenerative disease.
E435. The method of E434, in which the neurodegenerative disease is Alzheimer's disease, Parkinson's disease, Huntington's disease, amyotrophic lateral sclerosis (ALS), or age-related macular degeneration (AMD).
E436. The method of E433, in which the demyelinating disease is multiple sclerosis, neuromyelitis optica, acute disseminated encephalomyelitis, or transverse myelitis.
E437. The method of E414 or E418, in which the disease or condition that can be treated with erythropoietin or an erythropoiesis-stimulating agent is gastrointestinal dysmotility or in which the subject has or is at risk of developing gastrointestinal dysmotility.
E438. The method of E437, in which the gastrointestinal dysmotility is associated with an intestinal injury, abdominal trauma, an intestinal inflammatory condition, an intestinal infection, slow transit constipation, post-operative ileus, a neurodegenerative injury, a neurotraumatic injury, a congenital problem, or a malnutrition-malabsorption problem.
E439. The method of E438, in which the intestinal infection is a bacterial infection (e.g., an infection that leads to sepsis or bacteremia), peritonitis, or ascites.
E440. The method of E438, in which the intestinal inflammatory condition is inflammatory bowel disease, Crohn's disease, or ulcerative colitis.
E441. The method of E438, in which the slow transit constipation is chronic constipation, idiopathic constipation, constipation due to post-operative ileus, or constipation caused by opiate use.
E442. The method of E438, in which the congenital problem is gastroschisis, omphalocele, aganglionic megacolon, Hirschsprung's disease, chronic intestinal pseudo-obstruction, small left colon syndrome, anorectal anomalies, esophageal dysplasia and atresia, ectopic anus, congenital hernias, or internal anal sphincter achalasia.
E443. The method of E438, in which the malnutrition-malabsorption problem is associated with an intestinal injury, an abdominal trauma, an intestinal inflammatory condition, an intestinal infection, constipation, post-operative ileus, a neurodegenerative injury, a neurotraumatic injury, a congenital problem, Gaucher disease, refeeding syndrome, extremely low birth weight, cancer cachexia, infection, cancer, spinal cord dysfunction, spinal dysraphism, bifida, a tumor, central nervous system dysfunction, peripheral neuropathy, removal part of the gastrointestinal tract, hemorrhage, liver dysfunction, celiac disease, cystic fibrosis, muscular dystrophies, or cerebral palsy.
E444. The method of E414 or E418, in which the disease or condition that can be treated with erythropoietin or an erythropoiesis-stimulating agent is end-stage renal disease or in which the subject has or is at risk of developing end-stage renal disease.
E445. The method of E414 or E418, in which the disease or condition that can be treated with erythropoietin or an erythropoiesis-stimulating agent is polycythemia or in which the subject has or is at risk of developing polycythemia.
E446. The method of any one of E411-E445, in which the polypeptide, nucleic acid molecule, vector, construct, or pharmaceutical composition is administered to the subject prior to surgery (e.g., to increase red blood cell count prior to surgery), after stem cell transplantation, prior to or during a space flight, during or after tissue or organ transplantation, to promote the growth of new blood vessels, for granulation tissue formation, for trauma treatment, or for post-vascular graft treatment.
E447 The method of any one of E411-E446, in which the subject is receiving kidney dialysis.
E448. The method of any one of E411, E412, E414-E416, and E418-E447, in which the subject does not have anemia.
E449. The method of any one of E411, E412, E414-E416, and E418-E448, in which the subject has normal hematopoiesis.
E450. The method of any one of E411-E449, in which the subject has low serum erythropoietin.
E451. A method of preparing a tissue or organ for transplantation by contacting the tissue or organ (e.g., when in the tissue or organ donor or after removal from the tissue or organ donor) with a therapeutically effective amount of the polypeptide of any one of E1-E89, the nucleic acid molecule of E90, the vector of E91, the construct of E96 or E97, or the pharmaceutical composition of E94 or E95.
E452. The method of any one of E98-E410, wherein the method reduces or inhibits the binding of activin A, activin B and/or myostatin to their receptors (e.g., their endogenous receptors).
E453. The method of any one of E98-E130, E165-E192, and E452, wherein the polypeptide, nucleic acid, vector, construct, or pharmaceutical composition is administered in an amount sufficient to increase muscle mass and/or strength, increase lean mass, affect myostatin, activin A, activin B, and/or BMP9 signaling in the subject, or reduce or inhibit the binding of activin A, activin B and/or myostatin to their receptors (e.g., their endogenous receptors).
E454. The method of any one of E131-E213 and E452, wherein the polypeptide, nucleic acid, vector, construct, or pharmaceutical composition is administered in an amount sufficient to increase mineral bone density, reduce bone resorption, reduce bone loss, reduce the rate of bone resorption, increase bone formation, increase the rate of bone formation, reduce osteoclast activity, increase osteoblast activity, increase bone strength, reduce the risk or occurrence of bone fracture, affect myostatin, A, activin B, and/or BMP9 signaling in the subject, or reduce or inhibit the binding of activin A, activin B and/or myostatin to their receptors (e.g., their endogenous receptors).
E455. The method of any one of E214-E227 and E452, wherein the polypeptide, nucleic acid, vector, construct, or pharmaceutical composition is administered in an amount sufficient to reduce fibrosis, prevent the development of fibrosis, reduce the risk of developing fibrosis, delay the development of fibrosis, slow or inhibit the progression of fibrosis, treat fibrosis, reduce one or more symptom of fibrosis, improve the function of a fibrotic tissue or organ, affect myostatin, activin A, activin B, and/or BMP9 signaling in the subject, or reduce or inhibit the binding of activin A, activin B, and/or myostatin to their receptors (e.g., their endogenous receptors).
E456. The method of any one of E228-E259, E321-E354, and E452, wherein the polypeptide, nucleic acid, vector, construct, or pharmaceutical composition is administered in an amount sufficient to increase red blood cell levels, increase hemoglobin levels, increase red blood cell production, increase red blood cell count, increase hematocrit, reduce the need for a blood transfusion, increase the maturation and/or differentiation of erythroid progenitors (e.g., early and/or terminal stage erythroid progenitors), increase late-stage erythroid precursor maturation, recruit early-stage progenitors into the erythroid lineage, increase reticulocytes, increase proerythroblast numbers, increase reticulocyte numbers, reduce the accumulation of red blood cell progenitor cells, increase the number of early-stage erythroid precursors and/or progenitors, promote the progression of erythroid precursors and/or progenitors through erythropoiesis, treat anemia, affect myostatin, activin A, activin B, and/or BMP9 signaling in the subject, or reduce or inhibit the binding of activin A, activin B, and/or myostatin to their receptors (e.g., their endogenous receptors).
E457. The method of any one of E260-E293, E321-E354, and E452, wherein the polypeptide, nucleic acid, vector, construct, or pharmaceutical composition is administered in an amount sufficient to increase platelet levels, increase platelet production, increase platelet count, increase or induce megakaryocyte differentiation and/or maturation, reduce the accumulation of platelet progenitor cells, treat thrombocytopenia, affect myostatin, activin A, activin B, and/or BMP9 signaling in the subject, or reduce or inhibit the binding of activin A, activin B, and/or myostatin to their receptors (e.g., their endogenous receptors).
E458. The method of any one of E294-E354 and E452, wherein the polypeptide, nucleic acid, vector, construct, or pharmaceutical composition is administered in an amount sufficient to increase neutrophil levels, increase neutrophil production, increase neutrophil count, increase or induce the differentiation and/or maturation of progenitor cells into neutrophils, treat neutropenia, reduce susceptibility to infection, affect myostatin, activin A, activin B, and/or BMP9 signaling in the subject, or reduce or inhibit the binding of activin A, activin B, and/or myostatin to their receptors (e.g., their endogenous receptors).
E459. The method of any one of E355-E383 and E452, wherein the polypeptide, nucleic acid, vector, construct, or pharmaceutical composition is administered in an amount sufficient to prevent PH, reduce the risk of developing PH, reduce the severity or frequency of one or more symptoms of PH, delay the development of PH, slow or inhibit the progression of PH, treat PH, reduce pulmonary vascular remodeling, reduce vascular remodeling in the heart, reduce right ventricular hypertrophy, reduce pulmonary vascular resistance, improve performance in the 6 minute walk test, reduce bone loss, reduce pulmonary arterial muscularization or pulmonary arterial wall thickening, reduce right ventricular compensation, affect myostatin, activin A, activin B, and/or BMP9 signaling in the subject, or reduce or inhibit the binding of activin A, activin B, and/or myostatin to their receptors (e.g., their endogenous receptors).
E460. The method of any one of E384-E410 and E452, wherein the polypeptide, nucleic acid, vector, construct, or pharmaceutical composition is administered in an amount sufficient to reduce body fat, reduce the amount of subcutaneous fat, reduce the amount of visceral and/or hepatic fat, reduce adiposity, reduce the weights of epididymal and perirenal fat pads, reduce body fat percentage, reduce body weight, reduce the percentage of body weight gain, reduce fasting insulin levels, reduce blood glucose levels, increase insulin sensitivity, affect myostatin, activin A, activin B, and/or BMP9 signaling in the subject, reduce the proliferation of adipose cells, reduce or inhibit the binding of activin A, activin B, and/or myostatin to their receptors (e.g., their endogenous receptors), reduce LDL, reduce triglycerides, improve the serum lipid profile, regulate insulin biosynthesis and/or secretion from B-cells, delay, postpone, or reduce the need for insulin, or increase glucose clearance.
E461. The method of any one of E411-E452, wherein the composition is administered in an amount sufficient to increase EPO levels, increase EPO receptor levels, promote the growth of new blood vessels and/or the replacement of damaged vascular regions, promote granulation tissue formation, reduce infiltration of mononuclear cells into the brain of the subject, improve a neurological deficit, reduce axonal damage, reduce neuronal cell death, reduce glial cell death, affect myostatin, activin A, activin B, and/or BMP9 signaling in the subject, or reduce or inhibit the binding of activin A, activin B, and/or myostatin to their receptors.
E462. The method of any one of E98-E461, wherein the method does not cause a vascular complication in the subject.
E463. The method of E462, wherein the method does not increase vascular permeability or leakage.
E464. The method of any one of E98-E463, wherein the subject is a human.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the terms “extracellular activin receptor type II (ActRII) chimera,” “extracellular ActRII chimera,” and “ActRII chimera” refer to a peptide including amino acid sequence derived from both a soluble, extracellular portion of the single transmembrane receptor ActRIIB and a soluble, extracellular portion of the single transmembrane receptor ActRIIA. The ActRII chimeras result from the substitution of one or more amino acid sequence corresponding a β-sheet from one ActRII protein (e.g., ActRIIB) into the corresponding position of the other ActRII protein (e.g., ActRIIA) and/or from the substitution of one or more intervening sequence (e.g., a sequence between the β-sheets) from one ActRII protein (e.g., ActRIIB) into the corresponding position of the other ActRII protein (e.g., ActRIIA). For example, an ActRII chimera may be produced by replacing one or more amino acid sequence corresponding to a β-sheet in ActRIIB with an amino acid sequence corresponding to the β-sheet from ActRIIA. The extracellular ActRII chimera may also have an N-terminal truncation of 1-9 amino acids relative to the extracellular portion of ActRIIB or ActRIIA. The sequences of wild-type, human ActRIIB (SEQ ID NO: 32) and wild-type, human ActRIIA (SEQ ID NO: 33) are shown below, in which the signal peptide is italicized and the extracellular portion is bold.

Wild-type human ActRIIB (SEQ ID NO: 32):
MTAPWVALALLWGSLCAGSGRGEAETRECIYYNANWELERTNQSGLERCEGEQDKRLHCYAS
WRNSSGTIELVKKGCWLDDFNCYDRQECVATEENPQVYFCCCEGNFCNERFTHLPEAGGPEV
TYEPPPTAPTLLTVLAYSLLPIGGLSLIVLLAFWMYRHRKPPYGHVDIHEDPGPPPPSPLVGLKPL
QLLEIKARGRFGCVWKAQLMNDFVAVKIFPLQDKQSWQSEREIFSTPGMKHENLLQFIAAEKRG
SNLEVELWLITAFHDKGSLTDYLKGNIITWNELCHVAETMSRGLSYLHEDVPWCRGEGHKPSIAH
RDFKSKNVLLKSDLTAVLADFGLAVRFEPGKPPGDTHGQVGTRRYMAPEVLEGAINFQRDAFLRI
DMYAMGLVLWELVSRCKAADGPVDEYMLPFEEEIGQHPSLEELQEVVVHKKMRPTIKDHWLKH
PGLAQLCVTIEECWDHDAEARLSAGCVEERVSLIRRSVNGTTSDCLVSLVTSVTNVDLPPKESSI
Wild-type, human ActRIIA precursor protein (SEQ ID NO: 33):
MGAAAKLAFAVFLISCSSGAILGRSETQECLFFNANWEKDRTNQTGVEPCYGDKDKRRHCFAT
WKNISGSIEIVKQGCWLDDINCYDRTDCVEKKDSPEVYFCCCEGNMCNEKFSYFPEMEVTQPT
SNPVTPKPPYYNILLYSLVPLMLIAGIVICAFWVYRHHKMAYPPVLVPTQDPGPPPPSPLLGLKPL
QLLEVKARGRFGCVWKAQLLNEYVAVKIFPIQDKQSWQNEYEVYSLPGMKHENILQFIGAEKRG
TSVDVDLWLITAFHEKGSLSDFLKANVVSWNELCHIAETMARGLAYLHEDIPGLKDGHKPAISHR
DIKSKNVLLKNNLTACIADFGLALKFEAGKSAGDTHGQVGTRRYMAPEVLEGAINFQRDAFLRID
MYAMGLVLWELASRCTAADGPVDEYMLPFEEEIGQHPSLEDMQEVVVHKKKRPVLRDYWQKH
AGMAMLCETIEECWDHDAEARLSAGCVGERITQMQRLTNIITTEDIVTVVTMVTNVDFPPKESSL

An extracellular ActRII chimera may have a sequence described in Table 1 or Table 2.

As used herein, the term “N-terminal truncation” refers to a deletion of 1-9 amino acids (e.g., 1, 2, 3, 4, 5, 6, 7, 8, or 9 amino acids) from the N-terminus of an extracellular ActRII chimera (e.g., an extracellular ActRII chimera described herein, e.g., an extracellular ActRII chimera of Table 1 or Table 2, e.g., an extracellular ActRII chimera of any one of SEQ ID NOs: 96-126). The N-terminal truncation can remove amino acids up two to amino acids before the first cysteine (e.g., the two amino acids before the first cysteine (RE or QE) are retained in the N-terminally truncated ActRII chimeras).

As used herein, the term “linker” refers to a linkage between two elements, e.g., peptides or protein domains. A polypeptide described herein may include an extracellular ActRII chimera described herein (e.g., an extracellular ActRII chimera of Table 1 or Table 2, e.g., an extracellular ActRII chimera of any one of SEQ ID NOs: 96-126) fused to a moiety. The moiety may increase stability or improve pharmacokinetic properties of the polypeptide. The moiety (e.g., Fc domain monomer, an Fc domain, an albumin-binding peptide, a fibronectin domain, or a human serum albumin) may be fused to the polypeptide by way of a linker. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 1-200 amino acid sequence) occurring between two elements, e.g., peptides or protein domains, to provide space and/or flexibility between the two elements. An amino acid spacer is part of the primary sequence of a polypeptide (e.g., fused to the spaced peptides via the polypeptide backbone). The formation of disulfide bonds, e.g., between two hinge regions that form an Fc domain, is not considered a linker.

As used herein, the term “Fc domain” refers to a dimer of two Fc domain monomers. An Fc domain has at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the Fc domain (e.g., in a wild-type Fc domain), the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 antibody constant domain to reduce the interaction or binding between the Fc domain and an Fcγ receptor. In some embodiments, the Fc domain contains one or more amino acid substitutions that reduce or inhibit Fc domain dimerization. An Fc domain can be any immunoglobulin antibody isotype, including IgG, IgE, IgM, IgA, or IgD. Additionally, an Fc domain can be an IgG subtype (e.g., IgG1, IgG2a, IgG2b, IgG3, or IgG4). The Fc domain can also be a non-naturally occurring Fc domain, e.g., a recombinant Fc domain.

As used herein, the term “albumin-binding peptide” refers to an amino acid sequence of 12 to 16 amino acids that has affinity for and functions to bind serum albumin. An albumin-binding peptide can be of different origins, e.g., human, mouse, or rat. In some embodiments, an albumin-binding peptide has the sequence DICLPRWGCLW (SEQ ID NO: 88).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human red blood cell, platelet, neutrophil, or muscle cell).

As used herein, the term “fibronectin domain” refers to a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is a fibronectin type III domain (SEQ ID NO: 89) having amino acids 610-702 of the sequence of UniProt ID NO: P02751. In other embodiments, a fibronectin domain is an adnectin protein.

As used herein, the term “human serum albumin” refers to the albumin protein present in human blood plasma. Human serum albumin is the most abundant protein in the blood. It constitutes about half of the blood serum protein. In some embodiments, a human serum albumin has the sequence of UniProt ID NO: P02768 (SEQ ID NO: 90).

As used herein, the term “fused” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage. In some embodiments of a polypeptide described herein, an extracellular ActRII chimera described herein (e.g., an extracellular ActRII chimera of Table 1 or Table 2, e.g., an extracellular ActRII chimera of any one of SEQ ID NOs: 96-126) may be fused in tandem series to the N- or C-terminus of a moiety (e.g., Fc domain monomer (e.g., the sequence of SEQ ID NO: 34), an Fc domain (e.g., the sequence of SEQ ID NO: 87 or SEQ ID NO: 35), an albumin-binding peptide (e.g., the sequence of SEQ ID NO: 88), a fibronectin domain (e.g., the sequence of SEQ ID NO: 89), or a human serum albumin (e.g., the sequence of SEQ ID NO: 90)) by way of a linker. For example, an extracellular ActRII chimera is fused to a moiety (e.g., an Fc domain monomer, an Fc domain, an albumin-binding peptide, a fibronectin domain, or a human serum albumin) by way of a peptide linker, in which the N-terminus of the peptide linker is fused to the C-terminus of the extracellular ActRII chimera through a chemical bond, e.g., a peptide bond, and the C-terminus of the peptide linker is fused to the N-terminus of the moiety (e.g., Fc domain monomer, Fc domain, albumin-binding peptide, fibronectin domain, or human serum albumin) through a chemical bond, e.g., a peptide bond.

As used herein, the terms “bone mineral density (BMD),” “bone density,” and “bone mass” refer to a measure of the amount of bone mineral (e.g., calcium) in bone tissue. BMD may be measured by well-established clinical techniques known to one of skill in the art (e.g., by single-1 or dual-energy photon or X-ray absorptiometry). The concept of BMD relates to the mass of mineral per volume of bone, although clinically it is measured by proxy according to optical density per square centimeter of bone surface upon imaging. BMD measurement is used in clinical medicine as an indirect indicator of osteoporosis and fracture risk. In some embodiments, BMD test results are provided as a T-score, where the T-score represents the BMD of a subject compared to the ideal or peak bone mineral density of a healthy 30-year-old adult. A score of 0 indicates that the BMD is equal to the normal reference value for a healthy young adult. Differences between the measured BMD of subject and that of the reference value for a healthy young adult are measured in standard deviations units (SDs). Accordingly, a T-score of between +1 SD and −1 SD may indicate a normal BMD, a T-score of between −1 SD and −2.5 SD may indicate low bone mass (e.g., osteopenia), and a T-score lower than-2.5 SD may indicate osteoporosis or severe osteoporosis. In some embodiments, a polypeptide of the invention including an extracellular ActRII chimera described herein (e.g., an extracellular ActRII chimera of Table 1 or Table 2, e.g., an extracellular ActRII chimera of any one of SEQ ID NOs: 96-126), a nucleic acid encoding such a polypeptide, or a vector containing such a nucleic acid molecule is administered to a subject in need thereof, wherein the patient has low bone mass (e.g., a T-Score of between −1 SD and −2.5 SD). In some embodiments, a polypeptide of the invention including an extracellular ActRII chimera described herein (e.g., an extracellular ActRII chimera of Table 1 or Table 2, e.g., an extracellular ActRII chimera of any one of SEQ ID NOs: 96-126), a nucleic acid encoding such a polypeptide, or a vector containing such a nucleic acid molecule is administered to a subject in need thereof, wherein the patient has osteoporosis (e.g., a T-Score of less than-2.5 SD). In some embodiments, administration of a polypeptide of the invention including an extracellular ActRII chimera described herein (e.g., an extracellular ActRII chimera of Table 1 or Table 2, e.g., an extracellular ActRII chimera of any one of SEQ ID NOs: 96-126), a nucleic acid encoding such a polypeptide, or a vector containing such a nucleic acid molecule treats the subject by increasing their BMD. In some embodiments, administration of a polypeptide of the invention including an extracellular ActRII chimera described herein (e.g., an extracellular ActRII chimera of Table 1 or Table 2, e.g., an extracellular ActRII chimera of any one of SEQ ID NOs: 96-126), a nucleic acid encoding such a polypeptide, or a vector containing such a nucleic acid molecule increases the BMD of a subject resulting in an increase in the T-Score of the subject (e.g., resulting in an increase in the T-Score of the subject of 0.1 or more, 0.2 or more, 0.3 or more, 0.4 or more, 0.5 or more, 1.0 or more, or 2.0 or more).

As used herein, the term “bone strength” refers to a measurement of bone that is determined by bone quality in addition to bone mineral density. Bone quality is influenced by bone geometry, microarchitecture, and the properties of constituent tissues. Bone strength can be used to assess the bone's risk of fracture.

As used herein, the term “bone disease” refers to a condition characterized by bone damage (e.g., decreased bone mineral density, decreased bone strength, and/or bone loss). Such diseases or conditions may be caused by an imbalance in osteoblast and/or osteoclast activity (e.g., increased bone resorption or reduced bone formation). Bone diseases include primary osteoporosis, secondary osteoporosis, osteopenia, osteopetrosis, bone fracture, bone cancer or cancer metastasis-related bone loss (e.g., bone loss associated with multiple myeloma), Paget's disease, renal osteodystrophy, osteogenesis imperfecta, neuromuscular disease-related bone loss, burn-induced bone loss (e.g., bone loss associated with a burn injury), anorexia-related bone loss, treatment-related bone loss, diet-related bone loss, bone loss associated with the treatment of obesity, low gravity-related bone loss, and immobility-related bone loss.

As used herein, the term “neuromuscular disease-related bone loss” refers to bone loss that occurs in a subject having a neuromuscular disease. Poor bone health is often a significant problem for patients with neuromuscular disease. Deficiency of bone mineral density and increased incidence of bone fractures, for example, are a well-recognized clinical consequence of diseases such as DMD, ALS, and SMA.

As used herein, the terms “bone remodeling” or “bone metabolism” refer to the process for maintaining bone strength and ion homeostasis by replacing discrete parts of old bone with newly synthesized packets of proteinaceous matrix. Bone is resorbed by osteoclasts and is deposited by osteoblasts in a process called ossification. Osteocyte activity plays a key role in this process. Conditions that result in a decrease in bone mass, can either be caused by an increase in resorption, or a decrease in ossification. In a healthy individual, during childhood, bone formation exceeds resorption. As the aging process occurs, resorption exceeds formation. Bone resorption rates are also typically much higher in post-menopausal older women due to estrogen deficiency related to menopause.

As used herein, the terms “bone resorption” or “bone catabolic activity” refer to a process by which osteoclasts break down the tissue in bones and release the minerals, resulting in a transfer of the mineral (e.g., calcium) from bone tissue to the blood. Increased rates of bone resorption are associated with aging, including in post-menopausal women. High rates of bone resorption, or rates of bone resorption that exceed the rate of ossification, are associated with bone disorders, such as decreased bone mineral density, including osteopenia and osteoporosis, and can result in bone loss. In some embodiments, a polypeptide of the invention including an extracellular ActRII chimera described herein (e.g., an extracellular ActRII chimera of Table 1 or Table 2, e.g., an extracellular ActRII chimera of any one of SEQ ID NOs: 96-126), a nucleic acid encoding such a polypeptide, or a vector containing such a nucleic acid molecule is administered to a subject in need thereof to decrease bone resorption (e.g., decrease bone loss) in the subject (e.g., the amount or rate of bone resorption in the subject).

As used herein, the terms “bone formation,” “ossification,” “osteogenesis,” or “bone anabolic activity” refer to the process of forming new bone tissue by osteoblasts. In some embodiments, a polypeptide of the invention including an extracellular ActRII chimera described herein (e.g., an extracellular ActRII chimera of Table 1 or Table 2, e.g., an extracellular ActRII chimera of any one of SEQ ID NOs: 96-126), a nucleic acid encoding such a polypeptide, or a vector containing such a nucleic acid molecule is administered to a subject in need thereof, to increase bone formation (e.g., increase the amount or rate of bone formation or osteogenesis in the subject). Reduced rates of bone formation, or rates of bone formation that are exceeded by the rate of bone resorption, can result in bone loss.

As used herein, the terms “increasing” and “decreasing” refer to modulating resulting in, respectively, greater or lesser amounts, of function, expression, or activity of a metric relative to a reference. For example, subsequent to administration of a polypeptide of the invention including an extracellular ActRII chimera in a method described herein (e.g., an extracellular ActRII chimera of Table 1 or Table 2, e.g., an extracellular ActRII chimera of any one of SEQ ID NOs: 96-126), the amount of a marker of a metric (e.g., lean mass) as described herein may be increased or decreased in a subject relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “fibrosis” refers to the pathological process of excess formation of fibrous connective tissue. Fibrosis is characterized by fibroblast accumulation and collagen deposition in excess of normal deposition in any particular tissue. In response to inflammation or an injury to a tissue, nearby fibroblasts can migrate into the wound, proliferate, and produce large amounts of collagenous extracellular matrix. When fibrosis occurs in response to injury, the term “scarring” can be used as synonym. Fibrosis may occur in many tissues of the body, including, e.g., lungs, skin, liver, kidney, heart, eye, tendon, cartilage, pancreatic tissue, uterine tissue, neural tissue, testis, ovary, adrenal gland, artery, vein, bone marrow, colon, small and large intestine, biliary tract, and gut.

As used herein, the term “muscle disease” refers to a disease or condition involving muscle weakness or atrophy (e.g., skeletal muscle weakness or atrophy). Motor neurons may also be affected in subjects with a muscle disease. A muscle disease may be caused by a genetic mutation (e.g., a muscular dystrophy) or may result from another disease or condition (e.g., cancer cachexia). Muscle diseases include neuromuscular diseases (e.g., a muscular dystrophy, IBM, ALS, SMA, CMT, myasthenia gravis, or multiple sclerosis), sarcopenia, cachexia, disuse atrophy, treatment-related muscle loss or atrophy, hypotonia, muscle loss or atrophy associated with hypoxia, and muscle loss or atrophy associated with a burn injury.

As used herein, the terms “pulmonary hypertension” or “PH” refer to a disease characterized by an increase in blood pressure between the heart and lungs, which can include an increase in blood pressure in pulmonary arteries (pulmonary arterial hypertension), pulmonary veins, or pulmonary capillaries. Pulmonary hypertension can have a number of symptoms, shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting. PH also features reduced exercise tolerance and may lead to heart failure.

As used herein, the terms “pulmonary arterial hypertension” or “PAH” refer to a form of pulmonary hypertension characterized by a narrowing or obstruction in the small pulmonary arteries, often caused by scarring, and an increase in pulmonary arterial blood pressure. PAH is also known as WHO Group I PH. PAH can be diagnosed based on an increase in blood pressure in the pulmonary artery mean pulmonary arterial pressure above 25 mmHg at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, SMAD9, caveolin 1, KCNK3, or EIF2AK4). In 70% of familial PAH cases, mutations are located in the BMPR2 gene. Risk factors for the development of PAH include family history of PAH, drug use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus).

As used herein, the terms “venous pulmonary hypertension” and “venous PH” refer to a form of pulmonary hypertension that is secondary to left heart disease. Venous PH is also known as WHO Group II PH. Venous PH may be associated with or caused by left ventricular systolic dysfunction (e.g., failure of the left ventricle), left ventricular diastolic dysfunction, valvular heart disease (e.g., mitral valve or aortic valve disease), congenital cardiomyopathy, or congenital/acquired pulmonary venous stenosis.

As used herein, the terms “hypoxic pulmonary hypertension” and “hypoxic PH” refer to a form of pulmonary hypertension that is due to lung disease or chronic hypoxia. This form of PH is also known as WHO Group III PH. Hypoxic PH may be associated with or caused by chronic obstructive pulmonary disease (e.g., emphysema), interstitial lung disease, sleep-disordered breathing (e.g., sleep apnea), lung disease (e.g., pulmonary fibrosis), alveolar hypoventilation disorders, chronic e. sure to high altitude, or developmental abnormalities.

As used herein, the terms “thromboembolic pulmonary hypertension” and “thromboembolic PH” refer to a form of pulmonary hypertension that is related to chronic arterial obstruction (e.g., blood clots). Thromboembolic PH is also known as WHO Group IV PH. Thromboembolic PH may be associated with or caused by chronic thromboembolic pulmonary hypertension, or other pulmonary artery obstructions (e.g., pulmonary emboli, angiosarcoma, arteritis, congenital pulmonary artery stenosis, or parasitic infection).

As used herein, the terms “miscellaneous pulmonary hypertension” and “miscellaneous PH” refer to a form of pulmonary hypertension with unclear or multifactorial mechanisms. This form of PH is categorized as WHO Group V PH. Miscellaneous PH may be associated with or caused by a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or thyroid diseases), pulmonary tumoral thrombotic microangiopathy, fibrosing mediastinitis, chronic kidney failure, or segmental pulmonary hypertension.

As used herein, the terms “increase red blood cell levels” and “promote red blood cell formation” refer to clinically observable metrics, such as hematocrit, red blood cell counts, and hemoglobin measurements, and are intended to be neutral as to the mechanism by which such changes occur. The term “low red blood cell levels” as used herein refers to red blood cell counts, hematocrit, and hemoglobin measurements that are below the range of values that is considered normal for the subject's age and gender.

As used herein, the terms “red blood cell formation” and “red blood cell production” refer to the generation of red blood cells, such as the process of erythropoiesis in which red blood cells are produced in the bone marrow.

As used herein, the terms “increase platelet levels” and “promote platelet formation” refer to clinically observable metrics, such as platelet counts, and are intended to be neutral as to the mechanism by which such changes occur. The term “low platelet levels” as used herein refers to platelet counts that are below the range of values that is considered normal for the subject's age and gender. The terms “platelet formation” and “platelet production” refer to the generation of platelets, such as the process in which platelets are produced from megakaryocytes.

As used herein, the terms “increase neutrophil levels” and “promote neutrophil formation” refer to clinically observable metrics, such as neutrophil counts, and are intended to be neutral as to the mechanism by which such changes occur. The term “low neutrophil levels” as used herein refers to neutrophil counts that are below the range of values that is considered normal for the subject's age and gender. The terms “neutrophil formation” and “neutrophil production” refer to the generation of neutrophils such as the process in which neutrophils are produced in the bone marrow.

As used herein, the term “anemia” refers to any abnormality in hemoglobin or red blood cells that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels and can be diagnosed using routine tests, such as a complete blood count.

As used herein, the term “normal hematopoiesis” refers to the process by which the components of blood and blood plasma are produced, which includes the formation of red blood cells (erythrocytes), white blood cells (leukocytes, which includes the formation of lymphocytes, neutrophils, eosinophils, basophils, and macrophages), and platelets (thrombocytes). A subject has normal hematopoiesis if the production of these cells is not impaired and the number of these cells in the blood falls within a range accepted as normal by a medical professional. For example, the normal red blood cell (RBC) range for men is 4.7 to 6.1 million cells per microliter (mcL), the normal RBC range for women who are not pregnant is 4.2 to 5.4 million mcL, and the normal RBC range for children is 4.0 to 5.5 million mcL.

As used herein, the term “a disease or condition that can be treated with EPO or an ESA” refers to a disease or condition that is currently treated by administering EPO, recombinant EPO, an EPO mimetic, or another agent that increases EPO or EPO receptor levels, a disease or condition that could be expected to benefit from increasing EPO or EPO receptor levels based on studies performed in cell culture conditions, animal models, or human trials, or a disease or condition that is associated with low serum EPO. Such diseases and conditions include end-stage renal disease, renal insufficiency, kidney dialysis, spinal cord injury, an iron overload disorder (e.g., hemochromatosis), an inflammatory brain disease, gastrointestinal dysmotility, ischemia, and other diseases and conditions as described in U.S. Pat. Nos. 5,013,718, 7,745,387, 8,466,172, 8,729,030, and 10,695,402 and U.S. Patent Application Publication Nos. US20180303903A1 and US20170312268A1, each of which is hereby incorporated by reference.

As used herein, the term “low serum erythropoietin” refers to a level of serum erythropoietin that is below the normal range. Normal levels of erythropoietin range from 4 to 26 milliunits per liter (mU/mL).

“Hypercholesterolemia” is characterized by elevated concentrations of cholesterol in the blood.

By far the commonest form of primary hypercholesterolemia is polygenic hypercholesterolemia. Secondary hypercholesterolemias frequently occur in association with diabetes mellitus, nephrotic syndrome, hypothyroidism, and hepatic disorders.

“Endothelium-mediated chronic inflammatory disorders” are disorders or conditions of a human or animal body that derive from a defense response of the body and its tissues to harmful stimuli, with certain signal molecules altering the properties of endothelial cells so that, in concert with the activation of other cell types, leukocytes remain adherent to endothelial cells, finally penetrate into the tissue and there initiate inflammation. One example of an endothelium-mediated inflammation is leukocytic vasculitis. A central part is played in the activation of an endothelium-mediated inflammatory event by the transcription factor NF-κB. Another system leading to the development of endothelial cell-mediated chronic inflammations is the AGE-RAGE system.

“Endotheliosis” refers to degenerative and proliferative endothelial changes associated with non-thrombopenic purpura. “Reticuloendotheliosis” refers to diseases of the reticulohistiocytic system, such as reticulum, reticulosis, reticulohistiocytosis and Hand-Schüller-Christian disease.

“Myocardial ischemia” refers to bloodlessness or hypoperfusion, that is an impairment of the blood supply, of the muscular wall of the heart as a result of inadequate or absent arterial supply of blood.

A “cardiac infarct” or “myocardial infarct” is a necrosis of a localized region of the myocardium, which usually occurs as an acute event complicating chronic coronary heart disease.

“Coronary heart disease” or “ischemic heart disease” is a degenerative coronary disorder which, owing to a constriction or a closure of coronary vessels of the heart, leads to a reduced blood supply to the myocardium.

“Angina pectoris” refers to an acute coronary insufficiency or stenocardia which may be induced by an imbalance of the oxygen supply and oxygen demand associated with coronary heart disease, coronary spasms, impairments of blood flow, cardiac arrhythmias, hypertension, or hypotension.

“Raynaud's disease” refers to ischemic states which are caused by vasoconstriction, that is vessel spasms, and occurs episodically, usually in the arteries of the fingers. Primary Raynaud's disease is a purely functional impairment of the small vessels supplying the distal parts of the extremities, whereas secondary Raynaud's disease has another disease underlying it, for example an inflammation of vessels.

“Preeclampsia” is an endothelial and vascular disease of the maternal body and appears to be the effect of endotheliotropic substances from the placenta. Preeclampsia is a multisystem disorder which may lead to disturbances of function of numerous organs and be manifested by diverse symptoms. The impairments of blood supply which are typical of the disorder are the result of an increased vascular resistance, possibly with local variations in severity. It is regarded as confirmed that an endothelial dysfunction is the central component of the pathogenesis of preeclampsia.

“Renal failure” refers to the restricted ability of the kidneys to excrete substances normally excreted in the urine, and in advanced stages there is also loss of the ability to regulate the electrolyte, water and acid-base balance. Terminal renal failure is characterized by a collapse of the excretory and endocrine function of the kidneys.

“Heart failure” refers to a pathological state that is also referred to as myocardial insufficiency or weakness of the heart muscle. Heart failure is characterized by inadequate functioning of the heart, the heart no longer being capable of efficient delivery to comply with the requirements. Heart failure can be categorized according to various aspects. For example, according to the affected segment of the heart it is classified as right heart failure, left heart failure and failure on both sides (global failure). According to the stability of an equilibrium influenced by physiological and therapeutic mechanisms, a distinction is made between compensated and decompensated heart failure. Classification takes place into acute and chronic heart failure according to the time course. Causes of heart failure are, inter alia, myocardial infarction, cardiomyopathy, inborn or acquired cardiac defects, essential or pulmonary hypertension, cardiac arrhythmias, coronary heart disease or myocarditis.

The term “ischemia” refers to a reduction in blood flow. Ischemia is associated with a reduction in nutrients, including oxygen, delivered to tissues. Ischemia may arise due to conditions such as atherosclerosis, formation of a thrombus in an artery or vein, or blockage of an artery or vein by an embolus, vascular closure due to other causes, e.g., vascular spasm, etc. Such conditions may reduce blood flow, producing a state of hypoperfusion to an organ or tissue, or block blood flow completely. Other conditions that can produce ischemia include tissue damage due to trauma or injury, such as, e.g., spinal cord injury; viral infection, which can lead to, e.g., congestive heart failure, etc. The terms “ischemic conditions” and “ischemic disorders” refer to acute ischemic conditions including myocardial infarction, ischemic stroke, pulmonary embolism, perinatal hypoxia, circulatory shock including, e.g., hemorrhagic, septic, cardiogenic, etc., acute respiratory failure, etc., chronic ischemic conditions including atherosclerosis, chronic venous insufficiency, chronic heart failure, cardiac cirrhosis, macular degeneration, sleep apnea, Raynaud's disease, systemic sclerosis, nonbacterial thrombotic endocarditis, occlusive artery disease, angina pectoris, TIAs, chronic alcoholic liver disease, etc. Ischemic conditions may also result when individuals are placed under general anesthesia and can cause tissue damage in organs prepared for transplant.

The terms “hypoxia” and “hypoxic” refer to an environment with levels of oxygen below normal. Hypoxia may be induced in cells by culturing the cells in a reduced oxygen environment, or cells may be treated with compounds that mimic hypoxia. Determining oxygen levels that define hypoxia in cell culture is well within the skill in the art. The terms “hypoxic conditions” and “hypoxic disorders” include ischemic disorders (ischemic hypoxia) such as those listed above, wherein hypoxia results from reduced circulation; pulmonary disorders (hypoxic hypoxia) such as chronic obstructive pulmonary disease (COPD), severe pneumonia, pulmonary edema, hyaline membrane disease, and the like, wherein hypoxia results from reduced oxygenation of the blood in the lungs; liver or renal disease, cancer or other chronic illness, and altitude sickness, etc.

“Wound healing” refers to the physiological processes for regenerating damaged tissue and for closing a wound, especially formation of new connective tissue and capillaries. The wound healing may be primary wound healing (first intention healing), which is characterized by rapid and complication-free closure and substantially complete recovery as a result of minimal formation of new connective tissue between the edges of a wound, which have a good blood supply and are approximated where appropriate, of a clean wound. Wounds where the edges of the wound are further apart and, in particular, crushed or necrotic, and infected wounds, undergo delayed secondary wound healing (second intention healing) in which, as a result of an (a) bacterial inflammation, there is filling of the tissue defect with granulation tissue and extensive formation of scar tissue. Epithelialization starting from the edge terminates the wound healing. The wound healing is divided into three phases, namely latency phase, proliferative phase and repair phase. The latency phase in turn is divided into the oxidative phase with scab formation, especially in the first few hours after the wound occurred, and the absorptive phase with catabolic autolysis, which extends over a period of from one to three days after the wound occurred. The proliferative phase is characterized by anabolic repair with production of collagen by fibroblasts and occurs on the fourth to seventh day after the wound occurred. The repair phase occurs after the eighth day after the wound occurred and is characterized by transformation of the granulation tissue into a scar.

A “wound” refers to an interruption of the coherence of body tissues with or without loss of substance and caused by mechanical injury or physically caused cell damage. Types of wounds are mechanical wounds, thermal wounds, chemical wounds, radiation wounds and disease-related wounds. Mechanical wounds arise through traumatic violence and occur in particular as incision and puncture wounds, crushing, lacerating, tearing and abrading wounds, scratch and bite wounds and projective wounds. Thermal wounds arise through exposure to heat or cold. Chemical wounds arise in particular through the action of acids or alkalis. Radiation wounds arise for example through exposure to actinic and ionizing radiation. Wounds occurring in relation to disease are in particular congestion-related wounds, traumatic wounds, diabetic wounds, etc.

The term “inflammatory brain disease or disorder” as used herein refers to a brain disease or disorder caused by acute or chronic inflammatory responses in the central nervous system. Acute inflammatory responses in the brain include activation of microglia, appearance of dendritic cells, and the release of pro-inflammatory cytokines and chemokines in the central nervous system. Chronic inflammatory responses include long-standing activation of microglia and subsequent sustained release of inflammatory mediators. Such long-standing activation of microglia results in activation and proliferation of additional microglia, and further release of inflammatory factors. Examples of chronic inflammatory brain diseases or disorders include demyelinating diseases, such as multiple sclerosis, and neurodegenerative diseases, such as Alzheimer's disease (AD), Parkinson's disease (PD), Huntington's disease, amyotrophic lateral sclerosis (ALS), and age-related macular degeneration (AMD).

As used herein, the term “thrombocytopenia” refers to a condition in which the blood contains a lower than normal number of platelets, which may be due to a deficiency in platelet production, accumulation of platelets within an enlarged spleen, or the destruction of platelets. Normal blood platelet levels range from about 150,000 to 450,000 per microliter blood in humans. A platelet count of less than 150,000 platelets per microliter is lower than normal. Bleeding can occur after a relatively minor injury if the platelet count falls below 50,000 platelets per microliter of blood, and serious bleeding may occur without any recognized injury if the platelet count falls below 10,000 to 20,000 platelets per microliter of blood.

As used herein, the term “immune thrombocytopenia” is used herein to refer to any type of thrombocytopenia arising from an autoimmune response directed against an individual's own platelets. Immune thrombocytopenia includes primary immune thrombocytopenia, in which autoimmune response is the original cause for the decrease in the platelet counts, such as idiopathic thrombocytopenic purpura. Immune thrombocytopenia also includes secondary immune thrombocytopenia, in which the decrease in platelet counts is associated with one or more other diseases that cause an individual's body to generate an autoimmune response against its own platelets, such as systemic lupus erythematosus (SLE), antiphospholipid syndrome (APS), Evans syndrome, immune thyroid disease, leukemia (e.g., chronic lymphocytic leukemia or large granular T-lymphocyte lymphocytic leukemia), or chronic infection (e.g., with Helicobacter pylori, human immunodeficiency virus (HIV), or Hepatitis C).

As used herein, the term “neutropenia” refers to a condition in which the blood contains an abnormally low number of neutrophils. The typical lower limit of the neutrophil count is about 1500 cells per microliter of blood. Below this level, the risk of infection increases. Neutropenia severity is classified as: mild (1000 to 1500 neutrophils per microliter of blood), moderate (500 to 1000 neutrophils per microliter of blood), and severe (below 500 neutrophils per microliter of blood). Neutropenia has many causes, but they typically fall into two main categories: destruction or depletion of neutrophils faster than the bone marrow can produce new neutrophils, or reduced production of neutrophils in the bone marrow.

As used herein, the term “low transfusion burden” refers to a condition of a subject that has received less than four units of red blood cells (RBCs) within eight weeks (e.g., 3, 2, 1, or 0 units of RBCs within eight weeks) prior to treatment with an ActRII chimera described herein (e.g., an extracellular ActRII chimera of Table 1 or Table 2, e.g., an extracellular ActRII chimera of any one of SEQ ID NOs: 96-126). A subject with a low transfusion burden can be identified as having anemia based on measurements of mean hemoglobin concentration. A subject with a low transfusion burden and a mean hemoglobin concentration of less than 10.0 g/dL of two measurements performed at least one week apart prior to treatment with an ActRII chimera described herein (e.g., one measurement performed within one day prior to treatment and the other performed 7-28 days prior, not influenced by RBC transfusion within seven days of measurement) is defined as having anemia. In some embodiments, a subject with a low transfusion burden receives 1-3 units of RBCs (1-3 RBC transfusions) within eight weeks prior to treatment with an ActRII chimera described herein. In some embodiments, a subject with a low transfusion burden does not receive any units of RBCs (0 RBC transfusions) within eight weeks prior to treatment with an ActRII chimera described herein.

As used herein, the term “high transfusion burden” refers to a condition of a subject requiring greater than or equal to four units of RBCs (e.g., 4, 5, 6, 7, 8, or more units) within eight weeks prior to treatment with an ActRII chimera described herein (e.g., an extracellular ActRII chimera of Table 1 or Table 2, e.g., an extracellular ActRII chimera of any one of SEQ ID NOs: 96-126). A subject with a high transfusion burden can be identified as having anemia based on measurements of mean hemoglobin concentration. A subject with a high transfusion burden and a mean hemoglobin concentration of less than or equal to 9.0 g/dL is defined as having anemia.

As used herein, the term “ineffective hematopoiesis” refers to the failure to produce fully mature hematopoietic cells (e.g., the failure to produce red blood cells, platelets, and neutrophils). Ineffective hematopoiesis may be due to single or multiple defects, such as abnormal proliferation and/or differentiation of progenitor cells (e.g., an excessive production of progenitors that are unable to complete differentiation), that can lead to a hyperproliferation or a shortage of progenitor cells.

As used herein, the terms “erythropoiesis stimulating agent” and “ESA” refer to a class of drugs that act on the proliferation stage of red blood cell development by expanding the pool of early-stage progenitor cells. Examples of erythropoiesis-stimulating agents are epoetin alfa and darbepoetin alfa.

As used herein, the term “metabolic disease” refers to a disease, disorder, or syndrome that is related to a subject's metabolism, such as breaking down carbohydrates, proteins, and fats in food to release energy, and converting chemicals into other substances and transporting them inside cells for energy utilization and/or storage. Some symptoms of a metabolic disease include high serum triglycerides, high low-density cholesterol (LDL), low high-density cholesterol (HDL), and/or high fasting insulin levels, elevated fasting plasma glucose, abdominal (central) obesity, and elevated blood pressure. Metabolic diseases increase the risk of developing other diseases, such as cardiovascular disease. In the present invention, metabolic diseases include, but are not limited to, obesity, Type 1 diabetes, and Type 2 diabetes.

As used herein, the term “treatment-related metabolic disease” refers to a metabolic disease (e.g., obesity, Type 1 diabetes, or Type 2 diabetes) associated with a medication taken by the subject (e.g., a metabolic disease developed during treatment with the medication). The medication can be one that the subject continues to take, or one taken previously that led to the development of the metabolic disease. Medications associated with the development of obesity include glucocorticoids (e.g., corticosteroids, such as prednisone), selective serotonin reuptake inhibitors (SSRIs, e.g., paroxetine, mirtazapine, fluoxetine, escitalopram, sertraline), tricyclic antidepressants (e.g., amitriptyline), mood stabilizers (e.g., valproic acid, lithium), antipsychotics (e.g., olanzapine, chlorpromazine, clozapine), and diabetes medication (e.g., insulin, chlorpropamide). Medications associated with the development of diabetes include glucocorticoids (e.g., corticosteroids, which may cause glucocorticoid-induced diabetes mellitus), SSRIs, serotonin-norepinephrine reuptake inhibitors (SNRIs), mood stabilizers (e.g., lithium and valproic acid), and antipsychotics (e.g., olanzapine and clozapine). In some embodiments, the development of obesity may lead to the development of diabetes.

As used herein, the term “age-related metabolic disease” refers to a metabolic disease (e.g., obesity, Type 1 diabetes, or Type 2 diabetes) that develops with age. For example, the risk of diabetes increases with age and is more common in older adults, with approximately 25% of adults over 60 having diabetes. Adults can develop Type 2 diabetes or new-onset Type 1 diabetes. Rates of obesity also increase with age, with the highest rates of obesity in the United States occurring in adults aged 40-59 (with a prevalence of obesity of 45%). Aging also reduces the body's ability to burn fat, leading to increased fat surrounding internal organs.

As used herein, the term “percentage of body weight gain” refers to the percentage of gained body weight compared to a prior body weight of a subject at a prior time. The percentage of body weight gain can be calculated as follows:

100×[(body weight at a later time−body weight at a prior time)/(body weight at a prior time)] In the present invention, administration of a polypeptide including an extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding a polypeptide including such a polypeptide, or vector containing such a nucleic acid molecule to a subject can reduce the percentage of body weight gain of the subject.

As used herein, the term “appetite for food intake” refers to a subject's natural desire or need for food. The appetite for food intake of a subject can be monitored by measuring the amount of food consumed after the polypeptide including an extracellular ActRII chimera described herein is administered. In the present invention, administration of a polypeptide including an extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding such a polypeptide, or vector containing such a nucleic acid molecule to a subject does not affect the subject's appetite for food intake.

As used herein, the term “adiposity” refers to the fat stored in the adipose tissue of a subject. In the present invention, administration of a polypeptide including extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding such a polypeptide, or vector containing such a nucleic acid molecule to a subject can reduce the subject's adiposity without affecting lean mass.

As used herein, the term “epididymal and perirenal fat pads” refers to the tightly packed fat cells in the epididymis and around the kidney. In the present invention, administration of a polypeptide including an extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding such a polypeptide, or vector containing such a nucleic acid molecule to a subject can reduce the weights of epididymal and perirenal fat pads of the subject.

As used herein, the term “fasting insulin” refers to a subject's level of insulin while the subject has not had any food intake for a length of time (i.e., 12-24 hours). Fasting insulin level is used in diagnosing metabolic diseases. Fasting insulin level is also used as an indication of whether a subject is at the risk of developing a metabolic disease. Normally, in a subject suffering from Type 1 diabetes, the subject's fasting insulin level is low compared to that of a healthy subject. In a subject suffering from insulin resistance (i.e., Type 2 diabetes), the subject's fasting insulin level is high compared to that of a healthy subject. In the present invention, administration of a polypeptide including an extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding such a polypeptide, or vector containing such a nucleic acid molecule to a subject can modulate the subject's fasting insulin level.

As used herein, the term “rate of glucose clearance” refers to the rate at which glucose is being cleared from the blood. The rate of glucose clearance can be measured in a glucose tolerance test (GTT). In a GTT, a subject is given a certain amount of glucose and blood samples are taken afterward to determine how quickly it is cleared from the blood. The rate of glucose clearance can be used as a parameter in diagnosing and/or determining the risk of developing metabolic diseases such as obesity, diabetes, and insulin resistance.

As used herein, the term “serum lipid profile” refers to the measurement of the distribution of different types of lipids and lipoproteins in a subject's serum. Such measurement can be accomplished by a panel of blood tests. The types of lipids and lipoproteins in a subject's serum include, but are not limited to, cholesterol (e.g., high-density lipoprotein (HDL) and low-density lipoprotein (LDL)), triglyceride, and free fatty acid (FFA). The distribution of the different types of lipids and lipoproteins can be used as a parameter in diagnosing and/or determining the risk of developing metabolic diseases such as obesity, diabetes, and insulin resistance. High levels of cholesterol, especially low-density lipoprotein, is generally regarded as an indication or risk factor for developing certain metabolic diseases, or in some severe medical cases, cardiovascular diseases. In the present invention, administration of a polypeptide including an extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding such a polypeptide, or vector containing such a nucleic acid molecule to a subject improves the subject's serum lipid profile such that the levels of cholesterol (especially low-density lipoprotein) and triglyceride are lowered.

As used herein, the term “C-terminal extension” refers to the addition of one or more amino acids to the C-terminus of a polypeptide including an extracellular ActRII chimera described herein (e.g., an extracellular ActRII chimera of Table 1 or Table 2, e.g., an extracellular ActRII chimera of any one of SEQ ID NOs: 96-126, e.g., adding one or more amino acids to the C-terminus of an ActRII chimera). The C-terminal extension can be one or more amino acids, such as 1-6 amino acids (e.g., 1, 2, 3, 4, 5, 6 or more amino acids). The C-terminal extension may include amino acids from the corresponding position of wild-type ActRIIA or ActRIIB. Exemplary C-terminal extensions are the amino acid sequence NP (a two amino acid C-terminal extension) and the amino acid sequence NPVTPK (SEQ ID NO: 91) (a six amino acid C-terminal extension). Any amino acid sequence that does not disrupt the activity of the polypeptide can be used.

As used herein, the term “percent (%) identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis. Serum half-life comparisons can be made by comparing the serum half-life of Fc fusion proteins.

As used herein, the term “lean mass” refers to a component of body composition which includes, e.g., lean mass, body fat, and body fluid. Normally lean mass is calculated by subtracting the weights of body fat and body fluid from total body weight. Typically, a subject's lean mass is between 60% and 90% of totally body weight. In the present invention, administration of a polypeptide including an extracellular ActRII chimera described herein (e.g., an extracellular ActRII chimera of Table 1 or Table 2, e.g., an extracellular ActRII chimera of any one of SEQ ID NOs: 96-126), a nucleic acid molecule encoding such a polypeptide, or vector containing such a nucleic acid molecule to a subject increases the subject's lean mass.

As used herein, the term “affinity” or “binding affinity” refers to the strength of the binding interaction between two molecules. Generally, binding affinity refers to the strength of the sum total of non-covalent interactions between a molecule and its binding partner, such as an extracellular ActRII chimera and BMP9 or activin A. Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (K_{A}). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. The K_{D} of two interacting molecules may be determined using methods and techniques well known in the art, e.g., surface plasmon resonance. K_{D} is calculated as the ratio of k_{off}/kₒₙ.

As used herein, the term “muscle mass” refers to the primary component of lean mass. Muscle mass can be measured experimentally by measuring muscle weight.

As used herein, the term “neuromuscular disease” refers to a disease that affects voluntary or involuntary muscle function due to problems in the nerves and muscles, typically leading to muscle weakness. Exemplary neuromuscular diseases include amyotrophic lateral sclerosis (ALS), autonomic neuropathy, botulism, Charcot-Marie-Tooth disease (CMT), chronic inflammatory demyelinating polyradiculoneuropathy, congenital myasthenic syndrome, congenital myopathies, cramp-fasciculation syndrome, dermatomyositis, diabetic neuropathy, distal myopathies, dystrophinopathies, endocrine myopathies, focal muscular atrophies, glycogen storage disease type II, Guillain-Barre syndrome, hereditary spastic paraplegia, inclusion body myositis (IBM), Isaac's syndrome, Kearns-Sayre syndrome, Kennedy disease, Lambert-Eaton myasthenic syndrome, metabolic myopathies, metabolic neuropathies, mitochondrial myopathies, motor neuron diseases, multiple sclerosis, muscular dystrophy (e.g., Duchenne (DMD), Becker (BMD), myotonic (DM), facioscapulohumeral (FSHD), limb-girdle (LGMD), distal (DD), oculopharyngeal (OPMD), Emery-Dreifuss (EDMD), and congenital (e.g., MDC1A, MDC1B, MDC1C, FCMD, WWS, RSMD1, MEB, and UCMD)), myasthenia gravis, myotonic dystrophy, necrotizing myopathies, neuromyotonia, neuropathy of Friedreich's Ataxia, nutritional neuropathy, peripheral neuropathy, polymyositis, primary lateral sclerosis, Schwartz-Jampel Syndrome, small fiber neuropathy, spinal and bulbar muscular atrophy, spinal muscular atrophy, spinal muscular atrophy with respiratory distress type 1, spinocerebellar ataxia, stiff person syndrome, toxic neuropathy, and Troyer syndrome. A neuromuscular disease may be inherited in an autosomal dominant or recessive pattern or mutations may occur spontaneously.

As used herein, the phrase “affecting myostatin, activin A, activin B, and/or BMP9 signaling” means changing the binding of myostatin, activin A, activin B, and/or BMP9 to their receptors, e.g., ActRIIA, ActRIIB, and BMPRII (e.g., endogenous receptors). In some embodiments, a polypeptide including an extracellular ActRII chimera described herein (e.g., an extracellular ActRII chimera of Table 1 or Table 2, e.g., an extracellular ActRII chimera of any one of SEQ ID NOs: 96-126) reduces or inhibits the binding of myostatin, activin A, activin B, and/or BMP9 to their receptors, e.g., ActRIIA, ActRIIB, and BMPRII (e.g., endogenous ActRIIA and/or ActRIIB).

As used herein, the term “vascular complication” refers to a vascular disorder or any damage to the blood vessels, such as damage to the blood vessel walls. Damage to the blood vessel walls may cause an increase in vascular permeability or leakage. The term “vascular permeability or leakage” refers to the capacity of the blood vessel walls to allow the flow of small molecules, proteins, and cells in and out of blood vessels. An increase in vascular permeability or leakage may be caused by an increase in the gaps (e.g., an increase in the size and/or number of the gaps) between endothelial cells that line the blood vessel walls and/or thinning of the blood vessel walls.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including an extracellular ActRII chimera (e.g., an extracellular ActRII chimera of Table 1 or Table 2, e.g., an extracellular ActRII chimera of any one of SEQ ID NOs: 96-126) fused to an Fc domain monomer may form a homodimer through the interaction of two Fc domain monomers, which form an Fc domain in the homodimer.

As used herein, the term “heterodimer” refers to a molecular construct formed by two different macromolecules, such as proteins or nucleic acids. The two monomers may form a heterodimer by covalent bonds or non-covalent bonds. For example, a polypeptide described herein including an extracellular ActRII chimera (e.g., an extracellular ActRII chimera of Table 1 or Table 2, e.g., an extracellular ActRII chimera of any one of SEQ ID NOs: 96-126) fused to an Fc domain monomer may form a heterodimer through the interaction of two Fc domain monomers, each fused to a different ActRII chimera, which form an Fc domain in the heterodimer.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector of the invention or a pharmaceutical composition containing a polypeptide, nucleic acid, or vector of the invention effective in achieving the desired therapeutic effect in treating a patient having or at risk of developing a disease, such as a muscle disease, a condition involving weakness or atrophy of muscles (e.g., a neuromuscular disease, such as a muscular dystrophy, IBM, ALS, SMA, CMT, myasthenia gravis, or multiple sclerosis; sarcopenia; or cachexia), a disease or condition involving bone damage (e.g., osteoporosis, or a condition involving bone damage, e.g., primary osteoporosis, secondary osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss), a disease or condition involving low red blood cell levels (e.g., anemia or blood loss), a disease or condition involving low platelet levels (e.g., thrombocytopenia), a disease or condition involving low neutrophil levels (e.g., neutropenia), a disease or condition involving fibrosis, a metabolic disease, PH (e.g., PAH, venous PH, hypoxic PH, thromboembolic PH, or miscellaneous PH), or a disease or condition that can be treated with EPO or an ESA (e.g., end-stage renal disease, renal insufficiency, kidney dialysis, spinal cord injury, an iron overload disorder (e.g., hemochromatosis), an inflammatory brain disease, ischemia, or gastrointestinal dysmotility). In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition of the present invention includes pharmaceutically acceptable components that are compatible with the polypeptide, nucleic acid, or vector. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to the polypeptide including an extracellular ActRII chimera, the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, the term “treating and/or preventing” refers to the treatment and/or prevention of a disease or condition, e.g., a muscle disease (e.g., a neuromuscular disease, such as a muscular dystrophy, IBM, SMA, CMT, ALS, myasthenia gravis, or multiple sclerosis; sarcopenia; or cachexia), a bone disease (e.g., a disease or condition involving bone damage, e.g., osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss), a disease involving low blood cell levels (e.g., anemia or blood loss), a disease involving low platelet levels (e.g., thrombocytopenia), a disease involving low neutrophil levels (e.g., neutropenia), fibrosis, a metabolic disease, PH (e.g., PAH, venous PH, hypoxic PH, thromboembolic PH, or miscellaneous PH), or a disease or condition that can be treated with EPO or an ESA, such as end-stage renal disease, renal insufficiency, kidney dialysis, spinal cord injury, an iron overload disorder (e.g., hemochromatosis), an inflammatory brain disease, ischemia, or gastrointestinal dysmotility, using methods and compositions of the invention. Generally, treating a muscle, bone, low blood cell, low platelet, low neutrophil, metabolic, or fibrotic disease, PH, or a disease or condition that can be treated with EPO or an ESA occurs after a subject has developed the muscle, bone, low blood cell, low platelet, low neutrophil, metabolic, or fibrotic disease, PH, or the disease or condition that can be treated with EPO or an ESA and/or is already diagnosed with the muscle, bone, low blood cell, low platelet, low neutrophil, metabolic, or fibrotic disease, PH, or the disease or condition that can be treated with EPO or an ESA. Preventing a muscle, bone, low blood cell, low platelet, low neutrophil, metabolic, or fibrotic disease, PH, or a disease or condition that can be treated with EPO or an ESA refers to steps or procedures taken when a subject is at risk of developing the muscle, bone, low blood cell, low platelet, low neutrophil, metabolic, or fibrotic disease, PH, or a disease or condition that can be treated with EPO or an ESA. The subject may show signs or mild symptoms that are judged by a physician to be indications or risk factors for developing the muscle, bone, low blood cell, low platelet, low neutrophil, metabolic, or fibrotic disease, PH, or a disease or condition that can be treated with EPO or an ESA, have another disease or condition associated with the development of the muscle, bone, low blood cell, low platelet, low neutrophil, metabolic, or fibrotic disease, PH, or a disease or condition that can be treated with EPO or an ESA, be undergoing treatment that may cause anemia, thrombocytopenia, neutropenia, fibrosis, obesity or diabetes, a disease or condition that can be treated with EPO or an ESA, or loss of bone density (e.g., surgery, chemotherapy, or radiation), or have a family history or genetic predisposition to developing the muscle, bone, low blood cell, low platelet, low neutrophil, metabolic or fibrotic disease, PH, or a disease or condition that can be treated with EPO or an ESA but has not yet developed the disease or condition.

As used herein, the term “subject” refers to a mammal, e.g., preferably a human. Mammals include, but are not limited to, humans and domestic and farm animals, such as monkeys (e.g., a cynomolgus monkey), mice, rats, dogs, cats, horses, sheep, goats, rabbits, and cows, etc.

### BRIEF DESCRIPTION OF THE DRAWINGS

FIGS. 1A-1D are a series of graphs showing the effect of three ActRII chimeras on muscle mass compared to vehicle. FIG. 1A shows the effect on lean mass. Muscle mass was measured for the quadricep (FIG. 1B), the tibialis anterior (FIG. 1C), and the pectoralis (FIG. 1D). Statistical analysis was performed using one-way ANOVA and Dunnett's multiple comparisons test. Data are shown ±SEM, *p<0.05, **p<0.01, ***p<0.001, ****p<0.0001.

FIGS. 2A-2C are a series of graphs showing the effect of three ActRII chimeras on red blood cells, hemoglobin, and hematocrit compared to vehicle. The three ActRII chimeras increased red blood cells (FIG. 2A), hemoglobin (FIG. 2B), and hematocrit (FIG. 2C) compared to vehicle. Statistical analysis was performed using one-way ANOVA and Dunnett's multiple comparisons test. Data are shown ±SEM, *p<0.05, **p<0.01, ***p<0.001, ****p<0.0001.

FIGS. 3A-3D are a series of graphs showing that ActRII chimeras increased trabecular bone in the proximal femur. The effect of ActRII chimeras ActRIIA-B7 (SEQ ID NO: 100), ActRIIA-B1B4 (SEQ ID NO: 102), and ActRIIA-B1B7 (SEQ ID NO: 101) on bone volume fraction (FIG. 3A), trabecular thickness (FIG. 3B), trabecular number (FIG. 3C), and trabecular separation (FIG. 3D) were evaluated. Data are shown as average±SEM. Statistical analysis was performed using one-way ANOVA with a Dunnett posttest, *p≤0.05, **p<0.01, ***p<0.001, ****p<0.0001.

### DETAILED DESCRIPTION OF THE INVENTION

The invention features polypeptides that include an extracellular activin receptor type II (ActRII) chimera. In some embodiments, a polypeptide of the invention includes an extracellular ActRII chimera fused to a moiety (e.g., Fc domain monomer, an Fc domain, an albumin-binding peptide, a fibronectin domain, or a human serum albumin). A polypeptide including an extracellular ActRII chimera fused to an Fc domain monomer may also form a dimer (e.g., homodimer or heterodimer) through the interaction between two Fc domain monomers. The ActRII chimeras described herein may have reduced binding to bone morphogenetic protein 9 (BMP9) relative to the wild-type extracellular ActRIIB, or have weak binding affinity or no binding affinity to BMP9 compared to binding affinity to activins (e.g., activin A and/or activin B) and myostatin. The invention also includes methods of treating diseases and conditions involving weakness or atrophy of muscles by increasing muscle mass, lean mass, and/or muscle strength, methods of treating or preventing bone damage by increasing bone mineral density, increasing bone formation, or decreasing bone resorption, methods of treating or preventing fibrosis, methods of treating or preventing low blood cell levels (e.g., anemia or blood loss) by increasing red blood cell levels (e.g., red blood cell count, hemoglobin levels, or hematocrit), red blood cell production, or erythroid progenitor maturation and/or differentiation (e.g., the maturation and/or differentiation of early-stage or late (e.g., terminal) stage erythroid progenitors into proerythroblasts, reticulocytes, or red blood cells), late-stage precursor (erythroid precursor) maturation (e.g., terminal maturation, such as the maturation of reticulocytes into red blood cells or the maturation of erythroblasts into reticulocytes and/or red blood cells), by recruiting early-stage progenitors into the erythroid lineage, by reducing the accumulation of red blood cell progenitor cells (e.g., by stimulating progenitor cells to progress to maturation), by increasing the number of early-stage erythroid precursors and/or progenitors (e.g., by expanding the early-stage precursor and/or progenitor population to provide a continuous supply of precursors to replenish polychromatic erythroblasts and allow for a continuous supply of maturing reticulocytes), or by promoting the progression of erythroid precursors and/or progenitors through erythropoiesis, methods of treating or preventing low platelet levels (e.g., thrombocytopenia) by increasing platelet levels (e.g., platelet count, megakaryocyte differentiation and/or maturation, and/or platelet production) or by reducing the accumulation of platelet progenitor cells (e.g., by stimulating progenitor cells to progress to maturation), methods of treating or preventing low neutrophil levels (e.g., neutropenia) by increasing neutrophil levels (e.g., neutrophil count, e.g., neutrophil production) or differentiation and/or maturation of progenitor cells (e.g., myeloid progenitors, myeloblasts, or myelocytes) into neutrophils, methods of treating or preventing pulmonary hypertension (PH) (e.g., PAH, venous PH, hypoxic PH, thromboembolic PH, or miscellaneous PH), methods of treating or preventing a metabolic disease (e.g., obesity, Type 1 diabetes, or Type 2 diabetes), methods of treating diseases and conditions that can be treated with EPO or an ESA (e.g., a disease or condition that can be treated by increasing EPO or EPO receptor levels), or methods of affecting myostatin, activin A, activin B, and/or BMP9 signaling in a subject by administering to the subject a polypeptide including an extracellular ActRII chimera described herein.

### I. Extracellular Activin Receptor Type II Chimeras

Activin type II receptors are single transmembrane domain receptors that modulate signals for ligands in the transforming growth factor β (TGF-β) superfamily. Ligands in the TGF-β superfamily are involved in a host of physiological processes, such as muscle growth, vascular growth, cell differentiation, homeostasis, hematopoiesis, and osteogenesis. Examples of ligands in the TGF-β superfamily include, e.g., activin (e.g., activin A and activin B), inhibin, growth differentiation factors (GDFs) (e.g., GDF8, also known as myostatin, and GDF11), and bone morphogenetic proteins (BMPs) (e.g., BMP9).

Myostatin and activins are known to play a role in the regulation of skeletal muscle growth. For example, mice without myostatin show a large increase in skeletal muscle mass. Myostatin has also been implicated in promoting fibrosis. Mice lacking myostatin show a reduction in muscle fibrosis, and injection of myostatin-coated beads induces muscle fibrosis in mice. Mice overexpressing an activin subunit that leads to the production of diffusible activin A also exhibit fibrosis. In addition, activins are expressed abundantly in bone tissues and regulate bone formation by controlling both osteoblast and osteoclast functions. Activin A has been reported to be upregulated in bone disease and inhibits osteoblast activity. TGF-β signaling pathways also regulate hematopoiesis, with signaling pathways involving activins preventing the differentiation of red blood cell, platelet, and neutrophil progenitor cells in order to maintain progenitor cells in a quiescent state, and signaling pathways involving BMPs promoting differentiation of progenitor cells. Homeostasis of this process is essential to ensure that all cell types, including red cells, white cells, and platelets, are properly replenished in the blood. Relatedly, activin receptor ligand GDF11 has been found to be overexpressed in a mouse model of hemolytic anemia and associated with defects in red blood cell production. Elevated activin A has also been observed in clinical and experimental pulmonary hypertension. Furthermore, activins are highly expressed in adipose tissue, and increased myostatin levels and activin receptor levels have been observed in subcutaneous and visceral fat of obese mice. Additionally, myostatin has been shown to be elevated in skeletal muscle and plasma of obese and insulin resistant women, and both type I and type II activin receptors have been linked to pancreatic function and diabetes. These data suggest that increased signaling through activin receptors, either due to increased expression of activin receptor ligands (e.g., activin A, activin B, myostatin) or increased expression of activin receptors themselves, could contribute to a variety of diseases and conditions, including muscle atrophy or weakness, fibrosis, bone disease, anemia, thrombocytopenia, neutropenia, pulmonary hypertension, and metabolic disease. Methods that reduce or inhibit activin A, activin B, and/or myostatin signaling could, therefore, be used in the treatment of diseases and conditions involving muscle atrophy or weakness, fibrosis, bone damage, low red blood cell levels (e.g., anemia), low platelet levels (e.g., thrombocytopenia), low neutrophil levels (e.g., neutropenia), pulmonary hypertension (e.g., PAH, venous PH, hypoxic PH, thromboembolic PH, or miscellaneous PH), or metabolic disorders (e.g., obesity, Type 1 diabetes, or Type 2 diabetes).

There exist two types of activin type II receptors: ActRIIA and ActRIIB. Studies have shown that BMP9 binds ActRIIB with about 300-fold higher binding affinity than ActRIIA (see, e.g., Townson et al., J. Biol. Chem. 287:27313, 2012). ActRIIA-Fc is known to have a longer half-life compared to ActRIIB-Fc. The present invention describes extracellular ActRII chimeras that are constructed by combining portions of extracellular ActRIIA and ActRIIB. In some embodiments, the ActRII chimeras exhibit reduced BMP9 binding relative to wild-type extracellular ActRIIB, which can prevent or reduce disruption of endogenous BMP9 signaling. In some embodiments, the chimeras have properties of both ActRIIA (e.g., low binding affinity to BMP9, the ability to increase red blood cell levels, and/or longer serum half-life as an Fc fusion protein) and ActRIIB (e.g., strong binding affinity to activins A and B and/or the ability to increase muscle mass). In one example, the ActRII chimeras have reduced binding affinity for BMP9 compared to wild-type extracellular ActRIIB, and confer increases in lean mass, muscle mass, bone mineral density, and/or red blood cell levels (e.g., increase red blood cell production and/or red cell mass or volume), decreases in body weight and/or body fat, and/or treat a muscle disease (e.g., a neuromuscular disease, such as a muscular dystrophy, IBM, SMA, CMT, ALS, myasthenia gravis, or multiple sclerosis; sarcopenia; or cachexia), a bone disease (e.g., a disease or condition involving bone damage, e.g., osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss), anemia, thrombocytopenia, neutropenia, metabolic disease (e.g., obesity, Type 1 diabetes, or Type 2 diabetes), fibrosis, or PH.

The ActRII chimeras may exhibit similar or improved binding to activins (e.g., activin A and/or activin B) and/or myostatin compared to wild-type extracellular ActRIIA and/or ActRIIB, allowing them to compete with endogenous activin receptors for ligand binding and reduce or inhibit endogenous activin receptor signaling. Consequently, the chimeras can be used to treat disorders in which activin receptor signaling is elevated, such as a bone disease, a muscle disease, fibrosis, PH, a metabolic disease, thrombocytopenia, neutropenia, and/or anemia, leading to a reduction in bone resorption or osteoclast activity, an increase in bone formation or bone mineral density, an increase in muscle mass, lean mass, or muscle strength, a reduction in fibrosis (e.g., reduced fibrosis or a slowing or stopping of the progression of fibrosis), an increase red blood cell levels (e.g., an increase in hemoglobin levels, hematocrit, or red blood cell counts, e.g., an increase in red blood cell production and/or red cell mass or volume), an increase in the maturation and/or differentiation of erythroid progenitors (e.g., early-stage or late (e.g., terminal) stage erythroid progenitors, e.g., maturation and/or differentiation of early-stage erythroid progenitors, such as colony forming unit-erythroid cells (CFU-Es) and burst forming unit-erythroid cells (BFU-Es), into proerythroblasts, reticulocytes, or red blood cells), recruitment of early-stage progenitors into the erythroid lineage, an increase in late-stage erythroid precursor maturation (e.g., terminal maturation, such as the maturation of reticulocytes into red blood cells, or the maturation of erythroblasts into reticulocytes and/or red blood cells), a reduction the accumulation of red blood cell progenitor cells (e.g., by stimulating progenitor cells to progress to maturation), an increase in the number of early-stage erythroid precursors and/or progenitors (e.g., an expansion of the early-stage precursor and/or progenitor populations), progression of erythroid precursors and/or progenitors through erythropoiesis (e.g., through the erythropoiesis pathway), an increase in platelet levels (e.g., an increase in platelet count, megakaryocyte differentiation and/or maturation, and/or platelet production), a reduction in the accumulation of platelet progenitor cells (e.g., by stimulating progenitor cells to progress to maturation), an increase in neutrophil levels (e.g., an increase in neutrophil count, e.g., an increase in neutrophil production), an increase in the differentiation and/or maturation of progenitor cells (e.g., myeloid progenitors, myeloblasts, or myelocytes) into neutrophils, a reduction in the symptoms or progression of PH, or a reduction in body fat, body weight, blood glucose levels, or insulin resistance (e.g., leading to an increase in insulin sensitivity). Polypeptides including an ActRII chimera described herein can also be used to increase EPO and EPO receptor levels and, therefore, can be used as a replacement for EPO therapy. These polypeptides can be administered less frequently than current EPO therapies, which would greatly improve convenience for patients and could potentially reduce adverse effects. Accordingly, polypeptides including ActRII chimeras described herein can also be used to treat diseases or conditions that can be treated with EPO or an ESA.

The wild-type amino acid sequences of the extracellular portions of human ActRIIA and ActRIIB are shown below.

Human ActRIIA, extracellular portion (SEQ ID
NO: 30):
GAILGRSETQECLFFNANWEKDRTNQTGVEPCYGDKDKRRHCFATWKNI
SGSIEIVKQGCWLDDINCYDRTDCVEKKDSPEVYFCCCEGNMCNEKFSY
FPEMEVTQPTS
Human ActRIIB, extracellular portion (SEQ ID
NO: 31):
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPT

Polypeptides described herein include an extracellular ActRII chimera that contains sequence from both the extracellular portion of ActRIIB and the extracellular portion of ActRIIA. The ActRII chimeras result from the substitution of one or more amino acid sequence corresponding to a β-sheet and/or one or more intervening sequence (e.g., a sequence between the β-sheets), from one ActRII protein (e.g., ActRIIB) into the corresponding position of the other ActRII protein (e.g., ActRIIA). For example, an ActRII chimera may be produced by replacing one or more amino acid sequence corresponding to a β-sheet and/or one or more or an intervening sequence in ActRIIB with an amino acid sequence corresponding to the β-sheet or the intervening sequence from ActRIIA. An ActRII chimera may also be produced by replacing one or more amino acid sequence corresponding to a β-sheet and/or one or more intervening sequence in ActRIIA with an amino acid sequence corresponding to the β-sheet or the intervening sequence, from ActRIIB. In the ActRII chimeras, a β-sheet and/or an intervening sequence from one protein is replaced with the corresponding β-sheet and/or the corresponding intervening sequence from the other protein (e.g., the 5^{th} β-sheet from ActRIIA (β_{5A}) can be replaced with the 5^{th} β-sheet from ActRIIB (β_{5B})).

Each ActRII protein has seven β-sheets (β₁-β₇) and eight intervening sequences (X₁-X₈). The ActRII chimeras of the invention include at least one of Bia, β₂ₐ, β₃ₐ, β₄ₐ, β₅ₐ, or β₇ₐ and at least one of Bib, β_{2b}, β_{3b}, β_{4b}, β_{5b}, or β_{7b} or at least one of X₁ₐ, X₂ₐ, X₃ₐ, X₅ₐ, X₆ₐ, X₇ₐ, or X₈ₐ and at least one of X_{1b}, X_{2b}, X_{3b}, X_{5b}, X_{6b}, X_{7b}, or X_{8b}. Accordingly, an ActRII chimera may have one to five β-sheet substitutions (e.g., 1, 2, 3, 4, or 5 of β₁, β₂, β₃, β₄, β₅, and β₇ from one ActRII protein may be substituted with the corresponding β-sheet sequence from the other ActRII protein) and/or one to seven intervening sequence substitutions (e.g., 1, 2, 3, 4, 5, 6, or 7 of X₁, X₂, X₃, X₅, X₆, X₇, and X₈ from one ActRII protein may be substituted with the corresponding intervening sequence from the other ActRII protein). In some embodiments, the β-sheet sequence that is substituted is a minimal β-sheet sequence (e.g., at least HCFATWK (SEQ ID NO: 12), which is a portion of RHCFATWKNI (β₃ₐ) (SEQ ID NO: 11); at least HCYASWR (SEQ ID NO: 14), which is a portion of LHCYASWRNS (β_{3b}) (SEQ ID NO: 13); at least EIVKQGCW (SEQ ID NO: 16), which is a portion of SIEIVKQGCW (β₄ₐ) (SEQ ID NO: 15); at least ELVKKGCW (SEQ ID NO: 18), which is a portion of TIELVKKGCW (β_{4b}) (SEQ ID NO: 17); at least VE, which is a portion of VEK (β₅ₐ); at least V, which is a portion of VAT (β_{5b}); at least SYF, which is a portion of KFSYF (β₇ₐ) (SEQ ID NO: 26); or at least T, which is a portion of RFTHL (β_{7b}) (SEQ ID NO: 27)). The extracellular ActRII chimeras are the same length (e.g., have the same number of amino acids) as wild-type extracellular ActRIIA and ActRIIB, therefore, in embodiments in which minimal β-sheet sequences are substituted, contiguous amino acids from ActRIIA or ActRIIB are used to connect the minimal β-sheet to the neighboring intervening sequences to maintain the length (e.g., the number of amino acids) of the ActRII chimeras (e.g., to prevent the extracellular ActRII chimeras from having fewer amino acids than the corresponding regions of extracellular ActRIIA and ActRIIB). Exemplary ActRII chimera sequences are provided in Table 1 and Table 2.

The extracellular ActRII chimeras described herein may have comparable activity and/or binding affinity to wild-type extracellular ActRIIA or ActRIIB, or they may have improved activity or binding affinity relative to wild-type extracellular ActRIIA or ActRIIB. In some embodiments, the extracellular ActRII chimeras bind to activin A, activin B, myostatin, and/or GDF11 with sufficient affinity to compete with endogenous activin receptors for binding to one or more of these ligands. In some embodiments, the extracellular ActRII chimeras of the invention have reduced, weak, or no substantial binding to BMP9 (e.g., compared to wild-type ActRIIB).

In some embodiments, the extracellular ActRII chimeras described herein have an N-terminal truncation of 1-9 amino acids (e.g., 1, 2, 3, 4, 5, 6, 7, 8, or 9 amino acids). The N-terminal truncation can involve the removal of 1-9 amino acids from the N-terminus of any of the chimeras shown in Table 1 and Table 2. The N-terminal truncation can remove amino acids up two to amino acids before the first cysteine (e.g., the two amino acids before the first cysteine (RE or QE) are retained in the N-terminally truncated ActRII chimeras).

The extracellular ActRII chimeras of the invention may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the chimeras shown in Table 1 and Table 2. The C-terminal extension may correspond to sequence from the same position in wild-type ActRIIA or ActRIIB. For example, C-terminal extensions that can be included in the extracellular ActRII chimeras of the invention are the amino acid sequence NP and the amino acid sequence NPVTPK (SEQ ID NO: 91), which correspond to sequence found in the same position in wild-type ActRIIA.

TABLE 1
Extracellular ActRII chimeras of the invention
For the ActRII chimera having the sequence X₁β₁X₂β₂X₃β₃X₄β₄X₅β₅X₆β₆X₇β₇X₈:

| | | | |
|---|---|---|---|
| X1 | GAILGRSETQ (X₁ₐ) (SEQ ID NO: 1) | X5 | LDDINCYDRTDC (X₅ₐ) (SEQ ID NO: 19) |
| | or | | or |
| | GRGEAETR (X_{1b}) (SEQ ID NO: 2) | | LDDFNCYDRQEC (X_{5b}) (SEQ ID NO: 20) |
| β1 | ECLFF (β₁ₐ) (SEQ ID NO: 3) | B5 | VEK (β₅ₐ) or a portion thereof that |
| | or | | comprises VE |
| | ECIYY (β_{1b}) (SEQ ID NO: 4) | | or |
| | | | VAT (β_{5b}) or a portion thereof that |
| | | | comprises V |
| X2 | NANWEKDRTN (X₂ₐ) (SEQ ID NO: 5) | X6 | KDSPEV (X₆ₐ) (SEQ ID NO: 21) |
| | or | | or |
| | NANWELERTN (X_{2b}) (SEQ ID NO: 6) | | EENPQV (X_{6b}) (SEQ ID NO: 22) |
| β2 | QTGVEPC (β₂ₐ) (SEQ ID NO: 7) | B6 | YFCCCE (SEQ ID NO: 23) |
| | or | | |
| | QSGLERC (β_{2b}) (SEQ ID NO: 8) | | |
| X3 | YGDKDKR (X₃ₐ) (SEQ ID NO: 9) | X7 | GNMCNE (X₇ₐ) (SEQ ID NO: 24) |
| | or | | or |
| | EGEQDKR (X_{3b}) (SEQ ID NO: 10) | | GNFCNE (X_{7b}) (SEQ ID NO: 25) |
| β3 | RHCFATWKNI (β₃ₐ) (SEQ ID NO: 11) | β7 | KFSYF (β₇ₐ) (SEQ ID NO: 26) or a portion |
| | or a portion thereof that comprises | | thereof that comprises SYF |
| | HCFATWK (SEQ ID NO: 12) | | or |
| | or | | RFTHL (β_{7b}) (SEQ ID NO: 27) or a portion |
| | LHCYASWRNS (β_{3b}) (SEQ ID NO: | | thereof that comprises T |
| | 13) or a portion thereof that comprises | | |
| | HCYASWR (SEQ ID NO: 14) | | |
| X4 | SG | X8 | PEMEVTQPTS (X₈ₐ) (SEQ ID NO: 28) |
| | | | or |
| | | | PEAGGPEVTYEPPPTAPT (X_{8b}) (SEQ ID |
| | | | NO: 29) |
| β4 | SIEIVKQGCW (β₄ₐ) (SEQ ID NO: 15) | | |
| | or a portion thereof that comprises | | |
| | EIVKQGCW (SEQ ID NO: 16) | | |
| | or | | |
| | TIELVKKGCW (β_{4b}) (SEQ ID NO: 17) | | |
| | or a portion thereof that comprises | | |
| | ELVKKGCW (SEQ ID NO: 18) | | |

In some embodiments, the ActRII chimera has the sequence of an ActRII chimera listed in Table 2, below.

TABLE 2
Extracellular ActRII chimeras having the sequences of SEQ ID NOs: 96-126

| SEQ ID NO | Amino Acid Sequence |
|---|---|
| 96 | GAILGRSETQECLFFNANWEKDRTNQSGLERCYGDKDKRRHCFATWKNISGSIEIVKQGCWLDDI |
| | NCYDRTDCVEKKDSPEVYFCCCEGNMCNEKFSYFPEMEVTQPTS |
| 97 | GAILGRSETQECLFFNANWEKDRTNQTGVEPCYGDKDKRLHCYASWRNSSGSIEIVKQGCWLDD |
| | INCYDRTDCVEKKDSPEVYFCCCEGNMCNEKFSYFPEMEVTQPTS |
| 98 | GAILGRSETQECLFFNANWEKDRTNQTGVEPCYGDKDKRRHCFATWKNISGTIELVKKGCWLDDI |
| | NCYDRTDCVEKKDSPEVYFCCCEGNMCNEKFSYFPEMEVTQPTS |
| 99 | GAILGRSETQECLFFNANWEKDRTNQTGVEPCYGDKDKRRHCFATWKNISGSIEIVKQGCWLDDI |
| | NCYDRTDCVATKDSPEVYFCCCEGNMCNEKFSYFPEMEVTQPTS |
| 100 | GAILGRSETQECLFFNANWEKDRTNQTGVEPCYGDKDKRRHCFATWKNISGSIEIVKQGCWLDDI |
| | NCYDRTDCVEKKDSPEVYFCCCEGNMCNERFTHLPEMEVTQPTS |
| 101 | GAILGRSETQECIYYNANWEKDRTNQTGVEPCYGDKDKRRHCFATWKNISGSIEIVKQGCWLDDI |
| | NCYDRTDCVEKKDSPEVYFCCCEGNMCNERFTHLPEMEVTQPTS |
| 102 | GAILGRSETQECIYYNANWEKDRTNQTGVEPCYGDKDKRRHCFATWKNISGTIELVKKGCWLDDI |
| | NCYDRTDCVEKKDSPEVYFCCCEGNMCNEKFSYFPEMEVTQPTS |
| 103 | GAILGRSETQECLEFNANWEKDRTNQSGLERCYGDKDKRRHCFATWKNISGTIELVKKGCWLDDI |
| | NCYDRTDCVEKKDSPEVYFCCCEGNMCNEKFSYFPEMEVTQPTS |
| 104 | GRGEAETRECLFFNANWEKDRTNQTGVEPCYGDKDKRRHCFATWKNISGSIEIVKQGCWLDDIN |
| | CYDRTDCVEKKDSPEVYFCCCEGNMCNEKFSYFPEMEVTQPTS |
| 105 | GAILGRSETQECLFFNANWELERTNQTGVEPCYGDKDKRRHCFATWKNISGSIEIVKQGCWLDDI |
| | NCYDRTDCVEKKDSPEVYFCCCEGNMCNEKFSYFPEMEVTQPTS |
| 106 | GAILGRSETQECLFFNANWEKDRTNQTGVEPCEGEQDKRRHCFATWKNISGSIEIVKQGCWLDDI |
| | NCYDRTDCVEKKDSPEVYFCCCEGNMCNEKFSYFPEMEVTQPTS |
| 107 | GAILGRSETQECLFFNANWEKDRTNQTGVEPCYGDKDKRRHCFATWKNISGSIEIVKQGCWLDD |
| | FNCYDRQECVEKKDSPEVYFCCCEGNMCNEKFSYFPEMEVTQPTS |
| 108 | GAILGRSETQECLFFNANWEKDRTNQTGVEPCYGDKDKRRHCFATWKNISGSIEIVKQGCWLDDI |
| | NCYDRTDCVEKEENPQVYFCCCEGNMCNEKFSYFPEMEVTQPTS |
| 109 | GAILGRSETQECLFFNANWEKDRTNQTGVEPCYGDKDKRRHCFATWKNISGSIEIVKQGCWLDDI |
| | NCYDRTDCVEKKDSPEVYFCCCEGNFCNEKFSYFPEMEVTQPTS |
| 110 | GAILGRSETQECLFFNANWEKDRTNQTGVEPCYGDKDKRRHCFATWKNISGSIEIVKQGCWLDDI |
| | NCYDRTDCVEKKDSPEVYFCCCEGNMCNEKFSYFPEAGGPEVTYEPPPTAPT |
| 111 | GRGEAETRECIYYNANWELERTNQTGVEPCEGEQDKRLHCYASWRNSSGTIELVKKGCWLDDF |
| | NCYDRQECVATEENPQVYFCCCEGNFCNERFTHLPEAGGPEVTYEPPPTAPT |
| 112 | GRGEAETRECIYYNANWELERTNQSGLERCEGEQDKRRHCFATWKNISGTIELVKKGCWLDDFN |
| | CYDRQECVATEENPQVYFCCCEGNFCNERFTHLPEAGGPEVTYEPPPTAPT |
| 113 | GRGEAETRECIYYNANWELERTNQSGLERCEGEQDKRLHCYASWRNSSGSIEIVKQGCWLDDF |
| | NCYDRQECVATEENPQVYFCCCEGNFCNERFTHLPEAGGPEVTYEPPPTAPT |
| 114 | GRGEAETRECIYYNANWELERTNQSGLERCEGEQDKRLHCYASWRNSSGTIELVKKGCWLDDF |
| | NCYDRQECVEKEENPQVYFCCCEGNFCNERFTHLPEAGGPEVTYEPPPTAPT |
| 115 | GRGEAETRECIYYNANWELERTNQSGLERCEGEQDKRLHCYASWRNSSGTIELVKKGCWLDDF |
| | NCYDRQECVATEENPQVYFCCCEGNFCNEKFSYFPEAGGPEVTYEPPPTAPT |
| 116 | GRGEAETRECLFFNANWELERTNQSGLERCEGEQDKRLHCYASWRNSSGTIELVKKGCWLDDF |
| | NCYDRQECVATEENPQVYFCCCEGNFCNEKFSYFPEAGGPEVTYEPPPTAPT |
| 117 | GRGEAETRECLFFNANWELERTNQSGLERCEGEQDKRLHCYASWRNSSGSIEIVKQGCWLDDF |
| | NCYDRQECVATEENPQVYFCCCEGNFCNERFTHLPEAGGPEVTYEPPPTAPT |
| 118 | GRGEAETRECIYYNANWELERTNQTGVEPCEGEQDKRLHCYASWRNSSGSIEIVKQGCWLDDF |
| | NCYDRQECVATEENPQVYFCCCEGNFCNERFTHLPEAGGPEVTYEPPPTAPT |
| 119 | GRGEAETRECLFFNANWELERTNQTGVEPCEGEQDKRRHCFATWKNISGSIEIVKQGCWLDDEN |
| | CYDRQECVATEENPQVYFCCCEGNFCNERFTHLPEAGGPEVTYEPPPTAPT |
| 120 | GAILGRSETQECIYYNANWELERTNQSGLERCEGEQDKRLHCYASWRNSSGTIELVKKGCWLDD |
| | FNCYDRQECVATEENPQVYFCCCEGNFCNERFTHLPEAGGPEVTYEPPPTAPT |
| 121 | GRGEAETRECIYYNANWEKDRTNQSGLERCEGEQDKRLHCYASWRNSSGTIELVKKGCWLDDF |
| | NCYDRQECVATEENPQVYFCCCEGNFCNERFTHLPEAGGPEVTYEPPPTAPT |
| 122 | GRGEAETRECIYYNANWELERTNQSGLERCYGDKDKRLHCYASWRNSSGTIELVKKGCWLDDF |
| | NCYDRQECVATEENPQVYFCCCEGNFCNERFTHLPEAGGPEVTYEPPPTAPT |
| 123 | GRGEAETRECIYYNANWELERTNQSGLERCEGEQDKRLHCYASWRNSSGTIELVKKGCWLDDIN |
| | CYDRTDCVATEENPQVYFCCCEGNFCNERFTHLPEAGGPEVTYEPPPTAPT |
| 124 | GRGEAETRECIYYNANWELERTNQSGLERCEGEQDKRLHCYASWRNSSGTIELVKKGCWLDDF |
| | NCYDRQECVATKDSPEVYFCCCEGNFCNERFTHLPEAGGPEVTYEPPPTAPT |
| 125 | GRGEAETRECIYYNANWELERTNQSGLERCEGEQDKRLHCYASWRNSSGTIELVKKGCWLDDF |
| | NCYDRQECVATEENPQVYFCCCEGNMCNERFTHLPEAGGPEVTYEPPPTAPT |
| 126 | GRGEAETRECIYYNANWELERTNQSGLERCEGEQDKRLHCYASWRNSSGTIELVKKGCWLDDF |
| | NCYDRQECVATEENPQVYFCCCEGNFCNERFTHLPEMEVTQPTS |

In some embodiments, a polypeptide of the invention including an extracellular ActRII chimera (e.g., an extracellular ActRII chimera of Table 1 or Table 2, e.g., an extracellular ActRII chimera of any one of SEQ ID NOs: 96-126) may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRII chimera by way of a linker or other covalent bonds. A polypeptide including an extracellular ActRII chimera fused to an Fc domain monomer may form a dimer (e.g., homodimer or heterodimer) through the interaction between two Fc domain monomers, which combine to form an Fc domain in the dimer.

Furthermore, in some embodiments, a polypeptide described herein (e.g., an ActRII chimera-Fc fusion protein) has a serum half-life of at least 7 days in humans. The polypeptide may bind to activin A with a K_{D} of 1 pM or higher. In some embodiments, the polypeptide binds to activin A, activin B, and/or myostatin and exhibits reduced (e.g., weak) binding to BMP9 (e.g., compared to wild-type extracellular ActRIIB). In some embodiments, the polypeptide does not substantially bind to human BMP9.

In some embodiments, the polypeptide may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 300 PM and about 1 pM). In some embodiments, the polypeptide may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 200 PM and about 1 pM, or a K_{D} of less than 1 pM). The polypeptide may also bind to growth and differentiation factor 11 (GDF-11) with a K_{D} of approximately 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 200 pM and about 1 pM, or a K_{D} of less than 1 pM). The polypeptide may bind to GDF-8 with a K_{D} of approximately 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 5 pM). In some embodiments, the polypeptide may bind to human BMP9 with a K_{D} of about 1 pM or higher (e.g., a K_{D} of about 1, 5, 15, 30, 25, 30, 35, 40, 45, 50, 60, 70, 80, 90, 100, 200, 300, 400, 500, 600, 700, 800, or 900 pM or about 1, 2, 3, 4, 5, 6, 7, 8, 9, 10, 15, 20, 25, 30, 35, 40, 45, 50, 55, 60, 65, 70, 75, or 80 nM or higher, e.g., a K_{D} of 1 nM or higher). The polypeptide may also bind to human BMP10 with a K_{D} of about 1 pM or higher (e.g., a K_{D} of about 1, 5, 15, 30, 25, 30, 35, 40, 45, 50, 60, 70, 80, 90, 100, 200, 300, 400, 500, 600, 700, 800, or 900 pM or about 1, 2, 3, 4, 5, 6, 7, 8, 9, or 10 nM or higher).

### II. Fc Domains

In some embodiments, a polypeptide described herein may include an extracellular ActRII chimera (e.g., an extracellular ActRII chimera of Table 1 or Table 2, e.g., an extracellular ActRII chimera of any one of SEQ ID NOs: 96-126) fused to an Fc domain monomer of an immunoglobulin or a fragment of an Fc domain to increase the serum half-life of the polypeptide. A polypeptide including an extracellular ActRII chimera fused to an Fc domain monomer may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. An Fc domain (e.g., a wild-type Fc domain) forms the minimum structure that binds to an Fc receptor, e.g., FcγRI, FcγRIIa, FcγRIIb, FcγRIIIa, FcγRIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor. In some embodiments, an Fc domain is from an IgG1 antibody and includes amino acid substitutions L234A, L235A, and G237A. In some embodiments, an Fc domain is from an IgG1 antibody and includes amino acid substitutions D265A, K322A, and N434A. The aforementioned amino acid positions are defined according to Kabat (Sequences of Proteins of Immunological Interest, 5^{th} Ed. Public Health Service, National Institutes of Health, Bethesda, MD. (1991)). The Kabat numbering of amino acid residues may be determined for a given antibody by alignment at regions of homology of the sequence of the antibody with a “standard” Kabat numbered sequence. Furthermore, in some embodiments, an Fc domain does not induce any immune system-related response. For example, the Fc domain in a dimer of a polypeptide including an extracellular ActRII chimera fused to an Fc domain monomer may be modified to reduce the interaction or binding between the Fc domain and an Fcγ receptor. The sequence of an Fc domain monomer that may be fused to an extracellular ActRII chimera is shown below (SEQ ID NO: 34):

THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK

In some embodiments, an Fc domain is from an IgG1 antibody and includes amino acid substitutions L12A, L13A, and G15A, relative to the sequence of SEQ ID NO: 34. In some embodiments, an Fc domain is from an IgG1 antibody and includes amino acid substitutions D43A, K100A, and N212A, relative to the sequence of SEQ ID NO: 34. In some embodiments, the terminal lysine is absent from the Fc domain monomer having the sequence of SEQ ID NO: 34. In some embodiments, an extracellular ActRII chimera described herein may be fused to the N- or C-terminus of an Fc domain monomer (e.g., SEQ ID NO: 34) through conventional genetic or chemical means, e.g., chemical conjugation. If desired, a linker (e.g., a spacer) can be inserted between the extracellular ActRII chimera and the Fc domain monomer. The Fc domain monomer can be fused to the N- or C-terminus (e.g., C-terminus) of the extracellular ActRII chimera.

In some embodiments, a polypeptide described herein may include an extracellular ActRII chimera (e.g., an extracellular ActRII chimera of Table 1 or Table 2, e.g., an extracellular ActRII chimera of any one of SEQ ID NOs: 96-126) fused to an Fc domain. In some embodiments, the Fc domain contains one or more amino acid substitutions that reduce or inhibit Fc domain dimerization. In some embodiments, the Fc domain contains a hinge domain. The Fc domain can be of immunoglobulin antibody isotype IgG, IgE, IgM, IgA, or IgD. Additionally, the Fc domain can be an IgG subtype (e.g., IgG1, IgG2a, IgG2b IgG3, or IgG4). The Fc domain can also be a non-naturally occurring Fc domain, e.g., a recombinant Fc domain.

Methods of engineering Fc domains that have reduced dimerization are known in the art. In some embodiments, one or more amino acids with large side chains (e.g., tyrosine or tryptophan) may be introduced to the C_{H}3-C_{H}3 dimer interface to hinder dimer formation due to steric clash. In other embodiments, one or more amino acids with small side chains (e.g., alanine, valine, or threonine) may be introduced to the C_{H}3-C_{H}3 dimer interface to remove favorable interactions. Methods of introducing amino acids with large or small side chains in the C_{H}3 domain are described in, e.g., Ying et al. (J Biol Chem. 287:19399-19408, 2012), U.S. Patent Publication No. 2006/0074225, U.S. Pat. Nos. 8,216,805 and 5,731,168, Ridgway et al. (Protein Eng. 9:617-612, 1996), Atwell et al. (J Mol Biol. 270:26-35, 1997), and Merchant et al. (Nat Biotechnol. 16:677-681, 1998), all of which are incorporated herein by reference in their entireties.

In yet other embodiments, one or more amino acid residues in the C_{H}3 domain that make up the C_{H}3-C_{H}3 interface between two Fc domains are replaced with positively charged amino acid residues (e.g., lysine, arginine, or histidine) or negatively charged amino acid residues (e.g., aspartic acid or glutamic acid) such that the interaction becomes electrostatically unfavorable depending on the specific charged amino acids introduced. Methods of introducing charged amino acids in the C_{H}3 domain to disfavor or prevent dimer formation are described in, e.g., Ying et al. (J Biol Chem. 287:19399-19408, 2012), U.S. Patent Publication Nos. 2006/0074225, 2012/0244578, and 2014/0024111, all of which are incorporated herein by reference in their entireties.

In some embodiments of the invention, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y4071, K409E, K409D, K409T, and K4091, relative to the sequence of human IgG1. In some embodiments, the terminal lysine is absent from the Fc domain amino acid sequence. In one particular embodiment, an Fc domain includes the amino acid substitution T366W, relative to the sequence of human IgG1. The sequence of an exemplary Fc domain is shown in SEQ ID NO: 87, below:

DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

An exemplary sequence for an Fc domain lacking the terminal lysine is provided below (SEQ ID NO: 35):

DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG

### III. Albumin-Binding Peptide

In some embodiments, a polypeptide described herein may include an extracellular ActRII chimera (e.g., an extracellular ActRII chimera of Table 1 or Table 2, e.g., an extracellular ActRII chimera of any one of SEQ ID NOs: 96-126) fused to a serum protein-binding peptide. Binding to serum protein-binding peptides can improve the pharmacokinetics of protein pharmaceuticals.

As one example, albumin-binding peptides that can be used in the methods and compositions described herein are generally known in the art. In one embodiment, the albumin binding peptide includes the sequence DICLPRWGCLW (SEQ ID NO: 88).

In the present invention, albumin-binding peptides may be joined to the N- or C-terminus (e.g., C-terminus) of an extracellular ActRII chimera described herein to increase the serum half-life of the extracellular ActRII chimera. In some embodiments, an albumin-binding peptide is joined, either directly or through a linker, to the N- or C-terminus of an extracellular ActRII chimera.

In some embodiments, an extracellular ActRII chimera described herein may be fused to the N- or C-terminus of albumin-binding peptide (e.g., SEQ ID NO: 88) through conventional genetic or chemical means, e.g., chemical conjugation. If desired, a linker (e.g., a spacer) can be inserted between the extracellular ActRII chimera and the albumin-binding peptide. Without wishing to be bound by theory, it is expected that inclusion of an albumin-binding peptide in an extracellular ActRII chimera described herein may lead to prolonged retention of the therapeutic protein through its binding to serum albumin.

### IV. Fibronectin Domain

In some embodiments, a polypeptide described herein may include an extracellular ActRII chimera (e.g., an extracellular ActRII chimera of Table 1 or Table 2, e.g., an extracellular ActRII chimera of any one of SEQ ID NOs: 96-126) fused to fibronectin domains. Binding to fibronectin domains can improve the pharmacokinetics of protein pharmaceuticals.

Fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments of the present invention, a fibronectin domain is joined to the N- or C-terminus (e.g., C-terminus) of an extracellular ActRII chimera described herein to increase the serum half-life of the extracellular ActRII chimera. A fibronectin domain can be joined, either directly or through a linker, to the N- or C-terminus of an extracellular ActRII chimera.

As one example, fibronectin domains that can be used in the methods and compositions described here are generally known in the art. In one embodiment, the fibronectin domain is a fibronectin type III domain having amino acids 610-702 of the sequence of UniProt ID NO: P02751 (SEQ ID NO: 89, below):

GPVEVFITETPSQPNSHPIQWNAPQPSHISKYILRWRPKNSVGRWKEAT
IPGHLNSYTIKGLKPGVVYEGQLISIQQYGHQEVTRFDFTTTST

In another embodiment, the fibronectin domain is an adnectin protein.

In some embodiments, an extracellular ActRII chimera described herein may be fused to the N- or C-terminus of a fibronectin domain (e.g., SEQ ID NO: 89) through conventional genetic or chemical means, e.g., chemical conjugation. If desired, a linker (e.g., a spacer) can be inserted between the extracellular ActRII chimera and the fibronectin domain. Without wishing to be bound by theory, it is expected that inclusion of a fibronectin domain in an extracellular ActRII chimera described herein may lead to prolonged retention of the therapeutic protein through its binding to integrins and extracellular matrix components such as collagens and fibrins.

### V. Serum Albumin

In some embodiments, a polypeptide described herein may include an extracellular ActRII chimera (e.g., an extracellular ActRII chimera of Table 1 or Table 2, e.g., an extracellular ActRII chimera of any one of SEQ ID NOs: 96-126) fused to serum albumin. Binding to serum albumins can improve the pharmacokinetics of protein pharmaceuticals.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In preferred embodiments, serum albumin is human serum albumin. In some embodiments of the present invention, a human serum albumin is joined to the N- or C-terminus (e.g., C-terminus) of an extracellular ActRII chimera described herein to increase the serum half-life of the extracellular ActRII chimera. A human serum albumin can be joined, either directly or through a linker, to the N- or C-terminus of an extracellular ActRII chimera.

As one example, serum albumins that can be used in the methods and compositions described herein are generally known in the art. In one embodiment, the serum albumin includes the sequence of UniProt ID NO: P02768 (SEQ ID NO: 90, below):

MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

In some embodiments, an extracellular ActRII chimera described herein may be fused to the N- or C-terminus of a human serum albumin (e.g., SEQ ID NO: 90) through conventional genetic or chemical means, e.g., chemical conjugation. If desired, a linker (e.g., a spacer) can be inserted between the extracellular ActRII chimera and the human serum albumin. Without wishing to be bound by theory, it is expected that inclusion of a human serum albumin in an extracellular ActRII chimera described herein may lead to prolonged retention of the therapeutic protein.

### VI. Linkers

A polypeptide described herein may include an extracellular ActRII chimera described herein (e.g., an extracellular ActRII chimera of Table 1 or Table 2, e.g., an extracellular ActRII chimera of any one of SEQ ID NOs: 96-126) fused to a moiety by way of a linker. In some embodiments, the moiety increases stability of the polypeptide. Exemplary moieties include an Fc domain monomer, an Fc domain, an albumin-binding peptide, a fibronectin domain, or a human serum albumin. In the present invention, a linker between a moiety (e.g., an Fc domain monomer (e.g., the sequence of SEQ ID NO: 34), an Fc domain (e.g., SEQ ID NO: 87 or SEQ ID NO: 35), an albumin-binding peptide (e.g., SEQ ID NO: 88), a fibronectin domain (e.g., SEQ ID NO: 89), or a human serum albumin (e.g., SEQ ID NO: 90)) and an extracellular ActRII chimera described herein can be an amino acid spacer including 1-200 amino acids. Suitable peptide spacers are known in the art, and include, for example, peptide linkers containing flexible amino acid residues such as glycine, alanine, and serine. In some embodiments, a spacer can contain motifs, e.g., multiple or repeating motifs, of GA, GS, GG, GGA, GGS, GGG, GGGA (SEQ ID NO: 36), GGGS (SEQ ID NO: 37), GGGG (SEQ ID NO: 38), GGGGA (SEQ ID NO: 39), GGGGS (SEQ ID NO: 40), GGGGG (SEQ ID NO: 41), GGAG (SEQ ID NO: 42), GGSG (SEQ ID NO: 43), AGGG (SEQ ID NO: 44), or SGGG (SEQ ID NO: 45). In some embodiments, a spacer can contain 2 to 12 amino acids including motifs of GA or GS, e.g., GA, GS, GAGA (SEQ ID NO: 46), GSGS (SEQ ID NO: 47), GAGAGA (SEQ ID NO: 48), GSGSGS (SEQ ID NO: 49), GAGAGAGA (SEQ ID NO: 50), GSGSGSGS (SEQ ID NO: 51), GAGAGAGAGA (SEQ ID NO: 52), GSGSGSGSGS (SEQ ID NO: 53), GAGAGAGAGAGA (SEQ ID NO: 54), and GSGSGSGSGSGS (SEQ ID NO: 55). In some embodiments, a spacer can contain 3 to 12 amino acids including motifs of GGA or GGS, e.g., GGA, GGS, GGAGGA (SEQ ID NO: 56), GGSGGS (SEQ ID NO: 57), GGAGGAGGA (SEQ ID NO: 58), GGSGGSGGS (SEQ ID NO: 59), GGAGGAGGAGGA (SEQ ID NO: 60), and GGSGGSGGSGGS (SEQ ID NO: 61). In yet some embodiments, a spacer can contain 4 to 12 amino acids including motifs of GGAG (SEQ ID NO: 42), GGSG (SEQ ID NO: 43), e.g., GGAG (SEQ ID NO: 42), GGSG (SEQ ID NO: 43), GGAGGGAG (SEQ ID NO: 62), GGSGGGSG (SEQ ID NO: 63), GGAGGGAGGGAG (SEQ ID NO: 64), and GGSGGGSGGGSG (SEQ ID NO: 65). In some embodiments, a spacer can contain motifs of GGGGA (SEQ ID NO: 39) or GGGGS (SEQ ID NO: 40), e.g., GGGGAGGGGAGGGGA (SEQ ID NO: 66) and GGGGSGGGGSGGGGS (SEQ ID NO: 67). In some embodiments of the invention, an amino acid spacer between a moiety (e.g., an Fc domain monomer, an Fc domain, an albumin-binding peptide, a fibronectin domain, or a human serum albumin) and an extracellular ActRII chimera described herein may be GGG, GGGA (SEQ ID NO: 36), GGGG (SEQ ID NO: 38), GGGAG (SEQ ID NO: 68), GGGAGG (SEQ ID NO: 69), or GGGAGGG (SEQ ID NO: 70).

In some embodiments, a spacer can also contain amino acids other than glycine, alanine, and serine, e.g., AAAL (SEQ ID NO: 71), AAAK (SEQ ID NO: 72), AAAR (SEQ ID NO: 73), EGKSSGSGSESKST (SEQ ID NO: 74), GSAGSAAGSGEF (SEQ ID NO: 75), AEAAAKEAAAKA (SEQ ID NO: 76), KESGSVSSEQLAQFRSLD (SEQ ID NO: 77), GENLYFQSGG (SEQ ID NO: 78), SACYCELS (SEQ ID NO: 79), RSIAT (SEQ ID NO: 80), RPACKIPNDLKQKVMNH (SEQ ID NO: 81), GGSAGGSGSGSSGGSSGASGTGTAGGTGSGSGTGSG (SEQ ID NO: 82), AAANSSIDLISVPVDSR (SEQ ID NO: 83), or GGSGGGSEGGGSEGGGSEGGGSEGGGSEGGGSGGGS (SEQ ID NO: 84). In some embodiments, a spacer can contain motifs, e.g., multiple or repeating motifs, of EAAAK (SEQ ID NO: 85). In some embodiments, a spacer can contain motifs, e.g., multiple or repeating motifs, of proline-rich sequences such as (XP)ₙ (SEQ ID NO: 92), in which X may be any amino acid (e.g., A, K, or E) and n is from 1-5, and PAPAP (SEQ ID NO: 86).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

### VII. Vectors, Host Cells, and Protein Production

The polypeptides of the invention can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of a polypeptide of the invention may be prepared by a variety of methods known in the art. These methods include, but are not limited to, oligonucleotide-mediated (or site-directed) mutagenesis, PCR mutagenesis, ligation, and overlap extension PCR. A nucleic acid molecule encoding a polypeptide of the invention may be obtained using standard techniques, e.g., gene synthesis. Alternatively, a nucleic acid molecule encoding a wild-type extracellular ActRIIA or ActRIIB may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding a polypeptide of the invention may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7030, and HsS78Bst cells. In some embodiments, E. coli cells may also be used as host cells for the invention. Examples of E. coli strains include, but are not limited to, E. coli 294 (ATCC® 31,446), E. coli λ 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the polypeptides of the invention may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide of the invention may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultrafiltration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the polypeptides of the invention can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide of the invention. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### VIII. Pharmaceutical Compositions and Preparations

The invention features pharmaceutical compositions that include the polypeptides described herein (e.g., a polypeptide including an extracellular ActRII chimera, e.g., an extracellular ActRII chimera of Table 1 or Table 2, e.g., an extracellular ActRII chimera of any one of SEQ ID NOs: 96-126). In some embodiments, a pharmaceutical composition of the invention includes a polypeptide including an extracellular ActRII chimera described herein fused to a moiety (e.g., Fc domain monomer, or a dimer thereof, an Fc domain, an albumin-binding peptide, a fibronectin domain, or a human serum albumin) as the therapeutic protein. In some embodiments, a pharmaceutical composition of the invention including a polypeptide of the invention may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, a pharmaceutical composition of the invention includes a nucleic acid molecule (DNA or RNA, e.g., mRNA) encoding a polypeptide of the invention, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, arginine, and lysine, and carbohydrates such as glucose, mannose, sucrose, and sorbitol. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical compositions of the invention may be prepared in microcapsules, such as hydroxymethylcellulose or gelatin-microcapsule and poly-(methylmethacrylate) microcapsule. The pharmaceutical compositions of the invention may also be prepared in other drug delivery systems such as liposomes, albumin microspheres, microemulsions, nanoparticles, and nanocapsules. Such techniques are described in Remington: The Science and Practice of Pharmacy 22^{nd} edition (2012). The pharmaceutical compositions to be used for in vivo administration must be sterile. This is readily accomplished by filtration through sterile filtration membranes.

The pharmaceutical compositions of the invention may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polylactides, copolymers of L-glutamic acid and γ ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., a polypeptide of the invention, included in the pharmaceutical preparations is such that a suitable dose within the designated range is provided (e.g., a dose within the range of 0.01-100 mg/kg of body weight).

The pharmaceutical composition for gene therapy can be in an acceptable diluent or can include a slow release matrix in which the gene delivery vehicle is imbedded. If hydrodynamic injection is used as the delivery method, the pharmaceutical composition containing a nucleic acid molecule encoding a polypeptide described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### IX. Routes, Dosage, and Administration

Pharmaceutical compositions that include the polypeptides of the invention as the therapeutic proteins may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide of the invention or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides of the invention may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide described herein may be administered using hydrodynamic injection.

The dosage of the pharmaceutical compositions of the invention depends on factors including the route of administration, the disease to be treated, and physical characteristics, e.g., age, weight, general health, of the subject. A pharmaceutical composition of the invention may include a dosage of a polypeptide of the invention ranging from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 0.1-100 mg/kg, e.g., 0.5-50 mg/kg. Pharmaceutical compositions that include a polypeptide of the invention may be administered to a subject in need thereof, for example, one or more times (e.g., 1-10 times or more) daily, weekly, biweekly, every four weeks, monthly, every eight weeks, bimonthly, every twelve weeks, quarterly, every sixteen weeks, biannually, annually, or as medically necessary. In some embodiments, pharmaceutical compositions that include a polypeptide of the invention may be administered to a subject in need thereof weekly, biweekly, every four weeks, monthly, every eight weeks, bimonthly, every twelve weeks, quarterly, or every sixteen weeks. Dosages may be provided in either a single or multiple dosage regimens. The timing between administrations may decrease as the medical condition improves or increase as the health of the patient declines.

### X. Methods of Treatment

The invention is based on the discovery that combining extracellular portions of ActRIIA and ActRIIB can yield ActRII chimeras with altered properties (e.g., altered ligand binding properties) compared to wild-type extracellular ActRIIA and ActRIIB. The ActRII chimeras generated by combining extracellular portions of ActRIIA and ActRIIB may possess beneficial properties of both ActRIIB (e.g., an ability to increase muscle mass and strong binding affinity to activins A and B) and ActRIIA (e.g., reduced binding affinity to BMP9 and/or longer serum half-life as an Fc fusion protein (e.g., compared to ActRIIB-Fc), and/or an ability to increase red blood cell levels). As the ActRII chimeras contain extracellular portions of ActRIIA and ActRIIB, they will be soluble and able to compete with endogenous activin receptors by binding to and sequestering ligands (e.g., activins A and B, myostatin, GDF11) without activating intracellular signaling pathways. Therefore, the extracellular ActRII chimeras described herein can be used to treat diseases or conditions in which elevated activin signaling has been implicated in pathogenesis (e.g., diseases or conditions in which increased expression of activin receptors or activin receptor ligands has been observed). For example, myostatin has been implicated in promoting fibrosis, inhibiting skeletal muscle growth, and regulating bone homeostasis, and elevated myostatin has been observed in subcutaneous and visceral fat of obese mice and plasma of obese and insulin resistant women. In addition, activin A has been reported to be upregulated in bone disease, clinical and experimental pulmonary hypertension, adipose tissue, and subcutaneous and visceral fat of obese mice, and has been found to inhibit osteoblast activity and promote fibrosis. Another activin receptor ligand, GDF11, has been found to be overexpressed in a mouse model of hemolytic anemia and associated with defects in red blood cell production, and both type I and type II activin receptors have been linked to pancreatic function and diabetes. Without wishing to be bound by theory, a therapeutic agent that binds to activin receptor ligands (e.g., GDF11, myostatin, and/or activins) and reduces their binding to or interaction with endogenous activin receptors (e.g., by sequestering the endogenous ligands) may have therapeutic utility for treating or preventing a variety of diseases or conditions, such as a muscle disease, a bone disease, fibrosis, anemia, thrombocytopenia, neutropenia, a metabolic disease (e.g., obesity, Type 1 diabetes, or Type 2 diabetes), or PH (e.g., PAH, venous PH, hypoxic PH, thromboembolic PH, or miscellaneous PH). Polypeptides containing an ActRII chimera described herein can also increase EPO and EPO receptor levels. Accordingly, polypeptides containing an ActRII chimera described herein can be used therapeutically in place of recombinant EPO or an EPO mimetic and can be used to treat any disease or condition that would benefit from increasing EPO and/or EPO receptor levels.

The compositions and methods described herein can be used to treat and/or prevent (e.g., prevent the development of or treat a subject diagnosed with) medical conditions, e.g., a muscle disease (e.g., skeletal muscle weakness or atrophy), a bone disease, low red blood cell levels (e.g., low hemoglobin levels or low red blood cell count, e.g., anemia), fibrosis, thrombocytopenia (e.g., low platelet count), neutropenia (e.g., low neutrophil count), a metabolic disease (e.g., obesity, Type 1 diabetes, or Type 2 diabetes), or PH (e.g., PAH, venous PH, hypoxic PH, thromboembolic PH, or miscellaneous PH). In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126 (e.g., an effective amount of an ActRII chimera)) may be administered to increase muscle mass and strength in a subject in need thereof. In some embodiments, the polypeptides described herein may be administered to increase lean mass. The polypeptides described herein may increase muscle mass and/or lean mass compared to measurements obtained prior to treatment. In some embodiments, the subject may have or be at risk of developing a disease or condition that results in muscle weakness or atrophy (e.g., a neuromuscular disease, cachexia, sarcopenia, or treatment-related muscle loss or atrophy). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing a disease or condition involving weakness and atrophy of muscles.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126 (e.g., an effective amount of an ActRII chimera)) may be administered to increase bone mineral density, increase bone formation, increase bone strength, reduce the risk or occurrence of bone fracture, or reduce bone resorption in a subject in need thereof. The polypeptides described herein may increase bone mineral density, increase bone formation, or reduce bone resorption compared to measurements obtained prior to treatment. In some embodiments, the subject may have or be at risk of developing a disease that results in bone damage (e.g., osteoporosis or osteopenia). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing a disease or condition involving bone damage.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126 (e.g., an effective amount of an ActRII chimera)) may be administered to increase red blood cell levels (e.g., increase hemoglobin levels, increase red blood cell count, increase red blood cell volume, increase red cell mass, increase hematocrit, or increase red blood cell formation or production), increase the maturation and/or differentiation of erythroid progenitors (early or late (e.g., terminal) stage progenitors, e.g., early-stage erythroid progenitors, such burst-forming unit-erythroid cells (BFU-Es) and/or colony forming unit-erythroid cells (CFU-Es), e.g., increase the maturation and/or differentiation of BFU-Es and/or CFU-Es into proerythroblasts, reticulocytes, or red blood cells, e.g., increase proerythroblast and/or reticulocyte numbers), increase late-stage precursor (erythroid precursor) maturation (e.g., terminal maturation, such as the maturation of reticulocytes into red blood cells, or the maturation of erythroblasts into reticulocytes and/or red blood cells), recruit early-stage progenitors into the erythroid lineage, increase the number of early-stage erythroid precursors and/or progenitors (e.g., expand the early-stage precursor population to provide a continuous supply of precursors to replenish polychromatic erythroblasts and allow for a continuous supply of maturing reticulocytes), promote the progression of erythroid precursors and/or progenitors through erythropoiesis, and/or reduce the accumulation of red blood cell progenitor cells (e.g., by stimulating progenitor cells to progress to maturation) in a subject in need thereof. The polypeptides described herein may increase red blood cell levels, increase the maturation and/or differentiation of erythroid progenitors, increase late-stage erythroid precursor maturation, recruit early-stage progenitors into the erythroid lineage, increase the number of early-stage erythroid precursors, promote the progression of erythroid precursors through erythropoiesis, or reduce the accumulation of red blood cell progenitor cells compared to measurements obtained prior to treatment. In some embodiments, the subject may have a disease or condition associated with low red blood cell levels (e.g., anemia or blood loss). In some embodiments, the subject may have or be at risk of developing anemia or blood loss (e.g., the subject may have or be at risk of developing anemia due to other diseases or conditions, such as a myelodysplastic syndrome, myelofibrosis, chronic kidney disease, rheumatoid arthritis, ineffective hematopoiesis, cancer, or an inflammatory disease (e.g., Crohn's disease, SLE, or ulcerative colitis), or due to medical treatments, such as chemotherapy, radiation therapy, or surgery). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing a disease or condition involving low red blood cell levels.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) may be administered to increase platelet levels (e.g., increase platelet count), promote megakaryocyte differentiation and/or maturation (e.g., to produce platelets), reduce platelet progenitor accumulation, improve blood clotting, reduce bleeding events, reduce bleeding in the skin (e.g., petechiae or bruising), and/or promote or increase platelet formation or production in a subject in need thereof. The polypeptides described herein may increase platelet levels, promote megakaryocyte differentiation and/or maturation, reduce platelet progenitor accumulation (e.g., by stimulating progenitor cells to progress to maturation), improve blood clotting, reduce bleeding events, reducing bleeding in the skin, and/or promote or increase platelet formation or production compared to measurements obtained prior to treatment. In some embodiments, the subject may have a disease or condition associated with low platelet levels (e.g., thrombocytopenia). In some embodiments, a megakaryocyte can be contacted in vitro with a polypeptide described herein, a nucleic acid encoding the polypeptide, or a vector containing the nucleic acid to generate platelets for the treatment of thrombocytopenia. In some embodiments, the subject may have or be at risk of developing thrombocytopenia (e.g., the subject may have or be at risk of developing thrombocytopenia due to other diseases or conditions, such as a myelodysplastic syndrome, myelofibrosis, myelofibrosis treatment (e.g., treatment with a JAK inhibitor, such as with ruxolitinib, fedratinib, or pacritinib), ineffective hematopoiesis, Gaucher disease, aplastic anemia, Fanconi anemia, Diamond Blackfan anemia, Shwachman Diamond syndrome, heavy alcohol consumption, cirrhosis of the liver, cancer (e.g., leukemia or lymphoma), immune thrombocytopenia, an autoimmune disease (e.g., rheumatoid arthritis or lupus (e.g., SLE)), a viral infection (e.g., hepatitis C, HIV, chickenpox, mumps, rubella, parvovirus, or Epstein-Barr virus), a bacterial infection (e.g., bacteremia), vitamin deficiency (e.g., vitamin B-12 deficiency, folate deficiency, or iron deficiency), cancer treatment (e.g., chemotherapy or radiation therapy), an enlarged spleen, thrombotic thrombocytopenia purpura, idiopathic thrombocytopenia purpura, disseminated intravascular coagulation, hemolytic uremic syndrome, paroxysmal nocturnal hemoglobinuria, acquired amegakaryocytic thrombocytopenia, Pearson syndrome, dyskeratosis congenita, a genetic condition (e.g., Wiskott-Aldrich or May-Hegglin syndrome), dilution of platelets caused by blood transfusion, or a reduction of platelets caused by medication (e.g., heparin, quinine, a sulfa-containing antibiotic, such as vancomycin, rifampin, or trimethoprim, or an anticonvulsant, such as phenytoin)). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing a disease or condition involving low platelet levels.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) may be administered to increase neutrophil levels (e.g., increase neutrophil count), increase or promote the differentiation and/or maturation of progenitor cells (e.g., myeloid progenitors, myeloblasts, or myelocytes) into neutrophils, and/or promote or increase neutrophil formation or production in a subject in need thereof. The polypeptides described herein may increase neutrophil levels, increase or promote the differentiation and/or maturation of progenitor cells into neutrophils, and/or promote or increase neutrophil formation or production compared to measurements obtained prior to treatment. In some embodiments, the subject may have a disease or condition associated with low neutrophil levels (e.g., neutropenia). In some embodiments, the subject may have or be at risk of developing neutropenia (e.g., the subject may have or be at risk of developing neutropenia due to other diseases or conditions, such as a myelodysplastic syndrome, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer (e.g., leukemia), a vitamin deficiency (e.g., B-12 deficiency or folate deficiency), an enlarged spleen, an autoimmune disease (e.g., granulomatosis with polyangiitis, lupus (e.g., SLE), Evans syndrome, Felty syndrome, Crohn's disease, or rheumatoid arthritis), a viral infection (e.g., chickenpox, Epstein-Barr, Hepatitis A, Hepatitis B, Hepatitis C, HIV/AIDS, cytomegalovirus, Dengue fever, or measles), a bacterial infection (e.g., tuberculosis, salmonella infection, or sepsis), cancer treatment (e.g., chemotherapy or radiation therapy), or treatment with other medications (e.g., a medication used to treat overactive thyroid, such as methimazole and propylthiouracil; an antibiotic, such as vancomycin, penicillin G, trimethoprim, and oxacillin; an antiviral drug, such as ganciclovir and valganciclovir; an anti-inflammatory medication for ulcerative colitis or rheumatoid arthritis, such as sulfasalazine; a drug used to treat irregular heart rhythms, such as quinidine and procainamide; an anticonvulsant, such as phenytoin and valproate; an antipsychotic, such as clozapine; or levamisole). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing a disease or condition involving low neutrophil levels.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) may be administered to prevent or reduce fibrosis in a subject in need thereof. In some embodiments, the polypeptides described herein may be administered to slow or stop the progression of fibrosis, to reduce the risk of developing fibrosis, or to reduce (e.g., reduce the frequency or severity of) one or more symptom of fibrosis. The polypeptides described herein may reduce fibrosis or slow the progression of fibrosis by at least compared to the progression of fibrosis prior to treatment or compared to the progression of fibrosis in untreated subjects. In some embodiments, the subject may have or be at risk of developing fibrosis (e.g., the subject may have a disease or condition associated with fibrosis, such as a wound, hepatitis B or C, fatty liver disease, kidney disease (e.g., chronic kidney disease), heart disease, or atherosclerosis, or may be undergoing treatment associated with the development of fibrosis, such as chemotherapy, radiation, or surgery). In some embodiments, the polypeptides described herein prevent or delay the development of fibrosis in a subject at risk of developing fibrosis (e.g., a subject being treated with chemotherapy, radiation, or surgery, or a subject having a disease or condition associated with fibrosis, such as a wound, hepatitis B or C, fatty liver disease, kidney disease (e.g., chronic kidney disease), heart disease, or atherosclerosis). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing fibrosis or a disease or condition associated with fibrosis.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) may be administered to treat PH, reduce PH (e.g., reduce the severity or frequency of one or more symptoms of PH, such as shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting), prevent (e.g., prevent the development of) PH, reduce the risk of developing PH, or slow or stop the progression of PH in a subject in need thereof. The polypeptides described herein may reduce the symptoms of PH or slow the progression of PH compared to the symptoms or progression observed prior to treatment or compared to symptoms or progression of PH in untreated subjects. In some embodiments, the subject may have or be at risk of developing PH (e.g., the subject may have idiopathic PAH; the subject may have a disease or condition associated with PAH (e.g., a disease or condition that leads to increased risk of developing PAH), such as HIV infection, schistosomiasis, portal hypertension, pulmonary veno-occlusive disease, pulmonary capillary hemangiomatosis, cirrhosis of the liver, a congenital heart abnormality, a connective tissue/autoimmune disorder (e.g., scleroderma or lupus), or drug use or abuse (e.g., methamphetamine or cocaine use); the subject may have a family history of PH (e.g., heritable PAH); the subject may have a disease or condition associated with venous PH (e.g., a disease or condition that leads to increased risk of developing venous PH), such as left ventricular systolic dysfunction, left ventricular diastolic dysfunction, valvular heart disease, congenital cardiomyopathy, or congenital/acquired pulmonary venous stenosis; the subject may have a disease or condition associated with hypoxic PH (e.g., a disease or condition that leads to increased risk of developing hypoxic PH), such as chronic obstructive pulmonary disease (e.g., emphysema), interstitial lung disease, sleep-disordered breathing (e.g., sleep apnea), lung disease (e.g., pulmonary fibrosis), an alveolar hypoventilation disorder, chronic exposure to high altitude, or a developmental abnormality; the subject may have a disease or condition associated with thromboembolic PH (e.g., a disease or condition that leads to increased risk of developing thromboembolic PH), such as chronic thromboembolic pulmonary hypertension, or a pulmonary artery obstruction (e.g., a pulmonary embolism, angiosarcoma, arteritis, congenital pulmonary artery stenosis, or parasitic infection); or the subject may have a disease or condition associated with miscellaneous PH (e.g., a disease or condition that leads to increased risk of developing miscellaneous PH), such as a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or a thyroid disease), pulmonary tumoral thrombotic microangiopathy, fibrosing mediastinitis, chronic kidney failure, or segmental pulmonary hypertension (pulmonary hypertension restricted to one or more lobes of the lungs)). In some embodiments, the polypeptides described herein prevent or delay the development of PH in a subject at risk of developing PH (e.g., a subject with a family history of PH (e.g., heritable PAH), or a subject having a disease or condition that leads to increased risk of developing PAH, venous PH, hypoxic PH, thromboembolic PH, or miscellaneous PH. In some embodiments, the methods described herein are directed to affecting myostatin, activin A, activin B, and/or BMP9 signaling (e.g., reducing or inhibiting the binding of activin A, activin B, myostatin, and/or BMP9 to their receptors) in a subject having or at risk of developing PH or a disease or condition associated with PH. In some embodiments, the PH is PAH, venous PH, hypoxic PH, thromboembolic PH, or miscellaneous PH.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) may be administered to reduce body fat (e.g., amount of body fat or body fat percentage), reduce body weight or body weight gain, reduce fasting insulin levels, increase glucose clearance, reduce LDL, reduce triglycerides, improve serum lipid profile, or increase insulin sensitivity (e.g., reduce in insulin resistance) in a subject in need thereof. The polypeptides described herein may reduce body fat (e.g., amount of body fat or body fat percentage), reduce body weight or body weight gain, reduce fasting insulin levels, increase glucose clearance, reduce LDL, reduce triglycerides, improve serum lipid profile, or increase insulin sensitivity (e.g., reduce in insulin resistance) compared to measurements obtained prior to treatment. In some embodiments, the subject may have a disease or condition associated with obesity or diabetes (e.g., Type 1 or Type 2 diabetes). In some embodiments, the subject may have or be at risk of developing a metabolic disease (e.g., obesity, Type 1 diabetes, or Type 2 diabetes, e.g., the subject may be overweight, have a family history of obesity, have other medical conditions or risk factors linked to increased risk of developing obesity or diabetes (e.g., advanced age, or treatment with a glucocorticoid, a selective serotonin reuptake inhibitor (SSRI), a tricyclic antidepressant, a mood stabilizer, an antipsychotic, a serotonin-norepinephrine reuptake inhibitor (SNRI), or a diabetes medication), have a family history of diabetes, or have prediabetes). In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors) in a subject having or at risk of developing a metabolic disease (e.g., obesity, Type 1 diabetes, or Type 2 diabetes).

In some embodiments, a polypeptide including an extracellular ActRII chimera described herein (e.g., an extracellular ActRII chimera of Table 1 or Table 2, e.g., an extracellular ActRII chimera of any one of SEQ ID NOs: 96-126) reduces or inhibits the binding of myostatin, activin A, activin B, and/or BMP9 to their endogenous receptors, e.g., ActRIIA, ActRIIB, and/or BMPRII. The polypeptides described herein may reduce the binding of myostatin, activin A, activin B, and/or BMP9 to their endogenous receptors compared to the binding of myostatin, activin A, activin B, and/or BMP9 to their endogenous receptors in the absence of the polypeptides of the invention. In some embodiments, affecting myostatin, activin A, activin B, and/or BMP9 signaling (e.g., reducing or inhibiting the binding of myostatin, activin A, activin B, and/or BMP9 to their endogenous receptors, e.g., ActRIIA, ActRIIB, and/or BMPRII) results in an increase in the subject's muscle mass, an increase in the subject's lean mass, an increase in the subject's bone mineral density or bone formation, a decrease in the subject's bone resorption, an increase in the subject's red blood cell levels (e.g., hemoglobin levels, hematocrit, red blood cell count, red blood cell volume, or red cell mass, e.g., promotes or increases red blood cell formation or production), an increase the maturation and/or differentiation of erythroid progenitors, an increase in late-stage erythroid precursor maturation, recruitment of early-stage progenitors into the erythroid lineage, a reduction the accumulation of red blood cell progenitor cells, an increase the number of early-stage erythroid precursors and/or progenitors, progression of erythroid precursors and/or progenitors through erythropoiesis, an increase in the subject's platelet levels (e.g., an increase in platelet count, megakaryocyte differentiation and/or maturation, and/or platelet formation or production), a reduction in the accumulation of platelet progenitor cells, an improvement in blood clotting, a reduction in bleeding events, reduced bleeding in the skin, an increase in the subject's neutrophil levels (e.g., an increase in neutrophil count, e.g., an increase in neutrophil production or formation), an increase in the differentiation and/or maturation of progenitor cells into neutrophils, a reduction in the subject's fibrosis or risk of developing fibrosis, a delay in the development of fibrosis, a reduction (e.g., slowing or inhibiting) in the progression of fibrosis, a reduction body fat (e.g., amount of body fat or body fat percentage), a reduction in body weight or body weight gain, a reduction in fasting insulin levels, an increase in glucose clearance, an improvement in serum lipid profile, an increase in insulin sensitivity (e.g., a reduction in insulin resistance), a reduction in the symptoms of PH, a reduction in the risk of developing PH, a delay in the development of PH, and/or a reduction (e.g., slowing or inhibiting) in the progression of PH. The PH can be PAH, venous PH, hypoxic PH, thromboembolic PH, or miscellaneous PH.

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) may be administered to a subject to increase muscle mass or strength, to increase lean mass, to increase bone mineral density, to increase bone formation, to increase bone strength, to reduce the risk or occurrence of bone fracture, to decrease bone resorption, to increase red blood cell levels, to increase the maturation and/or differentiation of erythroid progenitors, to increase late-stage erythroid precursor maturation, to recruit early-stage progenitors into the erythroid lineage, to reduce the accumulation of red blood cell progenitor cells, to increase the number of early-stage erythroid precursors and/or progenitors, to promote progression of erythroid precursors and/or progenitors through erythropoiesis, to increase the subject's platelet levels, to increase megakaryocyte differentiation and/or maturation, to increase platelet formation or production, to reduce the accumulation of platelet progenitor cells, to improve blood clotting, to reduce bleeding events, to reduce bleeding in the skin, to increase the subject's neutrophil levels, to increase neutrophil production or formation, to increase or promote the differentiation and/or maturation of progenitor cells into neutrophils, to prevent or reduce fibrosis (e.g., to reduce fibrosis, to prevent or delay the development of fibrosis, or to slow or stop the progression of fibrosis), to treat a metabolic disease, to reduce body fat (e.g., amount of body fat or body fat percentage), to reduce body weight or body weight gain, to reduce fasting insulin levels, to increase glucose clearance, to improve serum lipid profile, to prevent or treat PH (e.g., to reduce symptoms of PH, to prevent or delay the development of PH, or to slow or stop the progression of PH), or to affect myostatin, activin A, activin B, and/or BMP9 signaling in the subject. The extracellular ActRII chimeras described herein (e.g., an extracellular ActRII chimera of Table 1 or Table 2, e.g., an extracellular ActRII chimera of any one of SEQ ID NOs: 96-126, e.g., an effective amount of an ActRII chimera) may increase muscle mass or strength, increase lean mass, increase bone mineral density, increase bone formation, increase bone strength, reduce the risk or occurrence of bone fracture, decrease bone resorption, increase red blood cell levels, increase the maturation and/or differentiation of erythroid progenitors, increase late-stage erythroid precursor maturation (e.g., terminal maturation, such as the maturation of reticulocytes into red blood cells, or the maturation of erythroblasts into reticulocytes and/or red blood cells), recruit early-stage progenitors into the erythroid lineage, reduce the accumulation of red blood cell progenitor cells, increase the number of early-stage erythroid precursors and/or progenitors, promote progression of erythroid precursors and/or progenitors through erythropoiesis, increase the subject's platelet levels, increase megakaryocyte differentiation and/or maturation, increase platelet formation or production, reduce the accumulation of platelet progenitor cells, improve blood clotting, reduce bleeding events, reduce bleeding in the skin, increase the subject's neutrophil levels, increase or promote the differentiation and/or maturation of progenitor cells into neutrophils, increase neutrophil production or formation, prevent or reduce fibrosis, treat a metabolic disease, reduce body fat (e.g., amount of body fat or body fat percentage), reduce body weight or body weight gain, reduce fasting insulin levels, increase glucose clearance, improve serum lipid profile, prevent or treat PH, or affect myostatin, activin A, activin B, and/or BMP9 signaling compared to measurements obtained prior to treatment or compared to measurements obtained from untreated subjects having the same disease or condition. In some embodiments, the methods described herein do not cause any vascular complications in the subject, such as increased vascular permeability or leakage.

The invention also includes methods of treating a subject having or at risk of developing a disease or condition involving weakness or atrophy of muscles by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, a subject having or at risk of developing a disease or condition involving weakness or atrophy of muscles has or is at risk of developing a disease or condition including a neuromuscular disease (e.g., a muscular dystrophy, IBM, SMA, CMT, ALS, myasthenia gravis, or multiple sclerosis), sarcopenia, cachexia (e.g., cancer cachexia, HIV-related cachexia, cardiac cachexia (e.g., cachexia associated with heart failure), cachexia associated with chronic kidney disease, or pulmonary cachexia (e.g., cachexia associated with COPD)), disuse atrophy; treatment related muscle loss or atrophy (e.g., glucocorticoid treatment, FGF-21 treatment, GLP-1 treatment, bariatric surgery, cancer therapy, or treatment for obesity or Type 2 diabetes), hypotonia, hypoxia, or muscle loss or atrophy associated with a burn injury. Muscular dystrophies include Duchenne muscular dystrophy (DMD), facioscapulohumeral muscular dystrophy (FSHD), Becker muscular dystrophy (BMD), myotonic dystrophy (DM), congenital muscular dystrophy, limb-girdle muscular dystrophy (LGMD), distal muscular dystrophy (DD), oculopharyngeal muscular dystrophy (OPMD), and Emery-Dreifuss muscular dystrophy (EDMD). There are thirty three types of congenital muscular dystrophies, which include congenital muscular dystrophy type 1A (MDC1A, associated with mutations in laminin alpha 2), congenital muscular dystrophy type 1C (MDC1C, associated with mutations in FKRP), congenital muscular dystrophy type 1D (MDC1D, associated with mutations in LARGE), congenital muscular dystrophy type 1B (MDC1B), Fukuyama congenital muscular dystrophy (FCMD, associated with mutations in fukutin), muscle-eye-brain disease (MEB, which may be associated with mutations in POMGnT1), Walker-Warburg Syndrome (WWS, associated with mutations in B3GNT1 (MDDGA type), POMT1 (MDDGA1 type), POMT2 (MDDGA2 type), ISPD (MDDGA7 type), GTDC2 (MDDGA8 type), TMEM5 (MDDGA10 type), B3GALNT2 (MDDGA11 type), or SGK196 (MDDGA12 type)), rigid spine muscular dystrophy (RSMD1, associated with a mutation in SEPN1), Ullrich congenital muscular dystrophy (UCMD, associated in mutations in COLGA1, COL6A2, or COL6A3), and muscular dystrophies associated with mutations in integrin alpha 7, integrin alpha 9, DOK7, laminin A/C, SBP2, or choline kinase beta. In some embodiments, the methods described herein increase muscle mass, e.g., increase muscle mass, lean mass, and/or muscle strength, e.g., increase muscle mass, lean mass, and/or muscle strength compared to measurements obtained prior to treatment or compared to measurements typically observed in untreated subjects having the same disease or condition. In some embodiments, the muscle is skeletal muscle. In some embodiments, the subject is identified as having a disease or condition that results in muscle weakness or atrophy prior to treatment with an ActRII chimera described herein. In some embodiments, the method includes a step of identifying the subject as having a disease or condition that results in muscle weakness or atrophy (e.g., by evaluating lean mass, muscle mass, or strength or by genetic testing for congenital muscular dystrophy) prior to treatment with an ActRII chimera described herein. The method can further include evaluating lean mass, muscle mass, or strength after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing a bone disease by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, a subject having or at risk of developing a bone disease (e.g., bone damage) has or is at risk of developing a disease or condition including primary osteoporosis, secondary osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, or immobility-related bone loss. In some embodiments, the primary osteoporosis is age-related or hormone-related osteoporosis (e.g., related to a decline in estrogen). In some embodiments, the secondary osteoporosis is immobilization-induced or glucocorticoid-induced (e.g., corticosteroid-induced) osteoporosis. Secondary osteoporosis may also result from endocrinopathies (e.g., Cushing's syndrome, thyrotoxicosis, hyperthyroidism, hypogonadism, hypopituitarism, primary hyperparathyroidism, diabetes mellitus, eating disorders, growth hormone deficiency, and acromegaly), gastrointestinal disorders (e.g., primary biliary cirrhosis, malabsorption syndrome, celiac disease, inflammatory bowel disease, gastric bypass surgery, hemochromatosis, and chronic liver diseases), hematological disorders (e.g., monoclonal gammopathy of uncertain significance, multiple myeloma, systemic mastocytosis, and beta thalassemia major), autoimmune disorders (e.g., rheumatoid arthritis, systemic lupus erythematosus, ankylosing spondylitis, and multiple sclerosis), renal disease (e.g., renal tubular acidosis, chronic kidney disease), medications (e.g., thyroid hormone, aromatase inhibitors, medroxyprogesterone acetate, GnRH agonists and antagonists, selective serotonin reuptake inhibitors, carbamazepine, phenytoin, cyclosporine, tacrolimus, antiretroviral therapy, lithium, heparin, furosemide and proton pump inhibitors), alcoholism, and transplantation. In some embodiments, the bone cancer is multiple myeloma or the cancer metastasis-related bone loss is caused by multiple myeloma. In some embodiments, the treatment-related bone loss occurs due to treatment with FGF-21 or GLP-1, due to treatment with an FGF-21 or GLP-1 containing therapeutic, due to treatment of Type 2 diabetes and/or obesity, due to bariatric surgery, due to androgen or estrogen deprivation therapy, or due to cancer therapy (e.g., chemotherapy or radiation). In some embodiments, the diet-related bone loss is rickets (e.g., vitamin D deficiency). In some embodiments, the low-gravity related bone loss is lack of load-related bone loss. In some embodiments, the methods described herein increase bone mineral density (e.g., increase bone mass), reduce bone resorption (e.g., reduce bone catabolic activity), increase bone formation (e.g., increase bone anabolic activity or increase osteogenesis), increase osteoblast activity or osteoblastogenesis, and/or decrease osteoclast activity or osteoclastogenesis, e.g., increase bone mineral density, reduce bone resorption, increase bone formation, increase osteoblast activity or osteoblastogenesis, and/or decrease osteoclast activity or osteoclastogenesis compared to measurements obtained prior to treatment or compared to measurements typically observed in untreated subjects having the same disease or condition. In some embodiments, the bone is cortical or trabecular bone. In some embodiments, the subject is identified as having a bone disease prior to treatment with an ActRII chimera described herein. In some embodiments, the method includes a step of identifying the subject as having a bone disease prior to treatment with an ActRII chimera described herein. The method can further include evaluating bone mineral density, bone formation, or bone resorption after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing anemia or blood loss by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, a subject having or at risk of developing low red blood cell levels (e.g., low hemoglobin levels, low hematocrit, or low red blood cell counts) has or is at risk of developing anemia or blood loss. In some embodiments, the anemia is associated with a nutritional deficit (e.g., a vitamin deficiency, such as vitamin B-12 deficiency or folate deficiency), a bone marrow defect (e.g., paroxysmal nocturnal hemoglobinuria), adverse reaction to medication (e.g., anti-retroviral HIV drugs), a myelodysplastic syndrome, bone marrow transplantation, myelofibrosis, ineffective hematopoiesis, cancer (e.g., a solid tumor, such as breast cancer, lung cancer, or colon cancer; a tumor of the lymphatic system, such as chronic lymphocytic leukemia, non-Hodgkin's lymphoma, or Hodgkin's lymphoma; or a tumor of the hematopoietic system, such as leukemia or multiple myeloma), cancer treatment (e.g., radiation or chemotherapy, e.g., chemotherapy with a platinum-containing agent), myelofibrosis treatment (e.g., treatment with a JAK inhibitor, such as ruxolitinib, fedratinib, or pacritinib), an inflammatory or autoimmune disease (e.g., rheumatoid arthritis, other inflammatory arthritides, systemic lupus erythematosus (SLE), an acute or chronic skin disease (e.g. psoriasis), or inflammatory bowel disease (e.g., Crohn's disease or ulcerative colitis), cystitis, gastritis), acute or chronic renal disease or failure (e.g., chronic kidney disease) including idiopathic and congenital conditions, diabetes, acute or chronic liver disease, acute or chronic bleeding, an infection (e.g., malaria, osteomyelitis), splenomegaly, porphyria, vasculitis, hemolysis, urinary tract infection, hemoglobinopathy (e.g., sickle cell disease), thalassemia (e.g., α- or β-thalassemia), Churg-Strauss syndrome, Felty syndrome, Pearson syndrome, dyskeratosis congenita, graft versus host disease, hematopoietic stem cell transplantation, osteomyelofibrosis, pancytopenia, pure red-cell aplasia, purpura Schoenlein-Henoch, Shwachman syndrome (e.g., Shwachman-Diamond syndrome), drug use or abuse (e.g., alcohol abuse), or contraindication to transfusion (e.g., in a patient of advanced age, a patient with allo- or auto-antibodies, a pediatric patient, a patient with cardiopulmonary disease, a patients who objects to transfusion for religious reasons (e.g., some Jehovah's Witnesses)). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del (5q)), myelodysplastic syndrome with excess blasts (MDS-EB; which includes myelodysplastic syndrome with excess blasts-type 1 (MDS-EB-1) and myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk MDS as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations, such a subject may have increased erythroid progenitor cells in the bone marrow relative to a healthy subject). In some embodiments, the MDS is associated with a defect in early-stage hematopoiesis (e.g., early-stage erythroid cell development, such as commitment or early differentiation, such a subject may have fewer erythroid progenitor cells in the bone marrow compared to a healthy subject or to a subject with a defect in terminal maturation). In some embodiments, the MDS is associated with elevated endogenous erythropoietin levels. In some embodiments, the myelodysplastic syndrome is associated with hypocellular bone marrow (e.g., a subject with MDS has hypocellular bone marrow). The subject may have a low transfusion burden or a high transfusion burden. In some embodiments, the subject has a low transfusion burden and received 1-3 RBC units in the eight weeks prior to treatment with an ActRII chimera described herein. In some embodiments, the subject has a low transfusion burden and did not receive a transfusion (received 0 RBC units) in the eight weeks prior to treatment with an ActRII chimera described herein. In some embodiments, the anemia is aplastic anemia, iron deficiency anemia, vitamin deficiency anemia, anemia of chronic disease (also called anemia of inflammation), anemia associated with bone marrow disease, hemolytic anemia, sickle cell anemia, microcytic anemia, hypochromic anemia, sideroblastic anemia, congenital dyserythropoietic anemia, Diamond Blackfan anemia, Fanconi anemia, or refractory anemia with excess of blasts. The sideroblastic anemia can be acquired sideroblastic anemia or congenital sideroblastic anemia. In some embodiments, the congenital sideroblastic anemia is associated with a mutation in ALAS2, SLC25A38, FECH, GLRX5, HSPA9, HSCB, SLC25A38, or ABC7. In some embodiments, the congenital sideroblastic anemia is associated with a mutation in PUS1, YARS2, LARS2, TRNT1, MT-ATP6, NDUFB11, or SLC19A2, or with an mtDNA mutation. The compositions and methods described herein can also be used to treat subjects that do not respond well to erythropoietin (EPO) or that are susceptible to adverse effects of EPO (e.g., hypertension, headaches, vascular thrombosis, influenza-like syndrome, obstruction of shunts, and myocardial infarction) or to treat subjects that do not respond to an erythroid maturation agent. In some embodiments, the subject has previously been treated with an ESA. In some embodiments, the subject has not previously been treated with an ESA. In some embodiments, the blood loss is due to surgery, trauma, a wound, an ulcer, urinary tract bleeding, digestive tract bleeding, frequent blood donation, or heavy menstrual bleeding (e.g., menorrhagia). In some embodiments, the methods described herein increase red blood cell levels (e.g., hemoglobin levels, hematocrit, red blood cell counts, red blood cell volume, and/or red cell mass), increase or induce red blood cell formation or production, increase the maturation and/or differentiation of erythroid progenitors (e.g., early-stage erythroid progenitors, such as BFU-Es and/or CFU-Es, e.g., increase the maturation and/or differentiation of BFU-Es and/or CFU-Es into proerythroblasts, reticulocytes, or red blood cells, e.g., increase proerythroblast and/or reticulocyte numbers), increase late-stage erythroid precursor maturation, recruit early-stage progenitors into the erythroid lineage, increase the number of early-stage erythroid precursors and/or progenitors, promote the progression of erythroid precursors and/or progenitors through erythropoiesis, and/or reduce the accumulation of red blood cell progenitor cells compared to measurements obtained prior to treatment or compared to measurements typically observed in untreated subjects having the same disease or condition. In some embodiments, the compositions and methods described herein reduce the need of a subject for a blood transfusion (e.g., reduce transfusion burden, for example, the subject no longer needs blood transfusions, or the subject needs less frequent blood transfusion than before treatment with the compositions and methods described herein). Subjects with normal red blood cell levels can also be treated using the methods and compositions described herein to increase red blood cell levels so that blood can be drawn and stored for later use in transfusions. In some embodiments, the compositions and methods described herein slow or inhibit the progression of lower-risk MDS to higher-risk MDS and/or acute myeloid leukemia (AML). For example, treatment of anemia in a subject having a very low, low, or intermediate risk MDS and a low transfusion burden may lead to a hemoglobin increase of greater than or equal to 1.5 g/dL from baseline or pretreatment measurements (e.g., for at least one day, two days, three days, four days, five days, six days, one week, two weeks, three weeks, one month, two months, or longer during treatment). In another example, treatment of anemia in a subject having a very low, low, or intermediate risk MDS and a high transfusion burden may lead to a reduction of ≥50% or ≥4 RBC units transfused compared to pretreatment (e.g., comparing an eight-week period during treatment to an eight-week period prior to treatment). In some embodiments, the subject is identified as having anemia (e.g., anemia associated with a myelodysplastic syndrome or myelofibrosis) prior to treatment with an ActRII chimera described herein. In some embodiments, the method includes a step of identifying the subject as having anemia (e.g., by evaluating red blood cell, hemoglobin, or hematocrit levels) prior to treatment with an ActRII chimera described herein. The method can further include evaluating red blood cell, hemoglobin, or hematocrit levels after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing thrombocytopenia by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, a subject having or at risk of developing low platelet levels (e.g., low platelet counts) has or is at risk of developing thrombocytopenia. In some embodiments, the thrombocytopenia is associated with a bone marrow defect, a myelodysplastic syndrome, bone marrow transplantation, myelofibrosis, myelofibrosis treatment (e.g., treatment with a JAK inhibitor, such as with ruxolitinib, fedratinib, or pacritinib), ineffective hematopoiesis, Gaucher disease, aplastic anemia, Fanconi anemia, Diamond Blackfan anemia, Shwachman Diamond syndrome, heavy alcohol consumption, cirrhosis of the liver, cancer (e.g., leukemia or lymphoma), an autoimmune disease (e.g., rheumatoid arthritis, lupus (e.g., SLE), antiphospholipid syndrome (APS), Evans syndrome, or immune thyroid disease), a viral infection (e.g., hepatitis C, HIV, chickenpox, mumps, rubella, parvovirus, or Epstein-Barr virus), a bacterial infection (e.g., bacteremia), an enlarged spleen, a vitamin deficiency (e.g., vitamin B-12 deficiency, folate deficiency, or iron deficiency), cancer treatment (e.g., chemotherapy or radiation therapy), thrombotic thrombocytopeniarpura, idiopathic thrombocytopeniarpura, disseminated intravascular coagulation, hemolytic uremic syndrome, paroxysmal nocturnal hemoglobinuria, or a reduction of platelets caused by medication (medication-induced thrombocytopenia, e.g., thrombocytopenia caused by treatment with heparin, quinine, a sulfa-containing antibiotic, such as vancomycin, rifampin, or trimethoprim, or an anticonvulsant, such as phenytoin)), acquired amegakaryocytic thrombocytopenia, Pearson syndrome, dyskeratosis congenita, dilution of platelets caused by blood transfusion, hematopoietic stem cell transplantation, or contraindication to transfusion (e.g., in a patient of advanced age, a patient with allo- or auto-antibodies, a pediatric patient, a patient with cardiopulmonary disease, a patient who objects to transfusion for religious reasons (e.g., some Jehovah's Witnesses)). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del (5q)), myelodysplastic syndrome with excess blasts (MDS-EB; which includes myelodysplastic syndrome with excess blasts-type 1 (MDS-EB-1) and myelodysplastic syndrome with excess blasts-type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk MDS as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations). In some embodiments, the MDS is associated with a defect in early-stage hematopoiesis (e.g., commitment or early differentiation). In some embodiments, the MDS is associated with elevated endogenous erythropoietin levels. In some embodiments, the myelodysplastic syndrome is associated with hypocellular bone marrow (e.g., the subject with MDS has hypocellular bone marrow). The subject may have a low transfusion burden or a high transfusion burden. In some embodiments, the subject has a low transfusion burden and received 1-3 RBC units in the eight weeks prior to treatment with an ActRII chimera described herein. In some embodiments, the subject has a low transfusion burden and did not receive a transfusion (received 0 RBC units) in the eight weeks prior to treatment with an ActRII chimera described herein. In some embodiments, the subject does not respond well to erythropoietin (EPO) or is susceptible to adverse effects of EPO (e.g., hypertension, headaches, vascular thrombosis, influenza-like syndrome, obstruction of shunts, and myocardial infarction). The compositions and methods described herein can also be used to treat subjects that do not respond to an erythroid maturation agent. In some embodiments, the subject has previously been treated with an ESA. In some embodiments, the subject has not previously been treated with an ESA. In some embodiments, the thrombocytopenia is familial thrombocytopenia (also referred to as inherited thrombocytopenia, e.g., thrombocytopenia associated with a genetic mutation, such as May-Hegglin anomaly, Sebastian syndrome, Fechtner syndrome, Epstein's syndrome, Wiskott-Aldrich syndrome, congenital amegakaryocytic thrombocytopenia, platelet storage pool deficiency, Hermansky-Pudlak syndrome, Bernard-Soulier syndrome, Von Willebrand Disease Type 2B, ANKRD26-related thrombocytopenia, thrombocytopenia absent radius syndrome, familial platelet disorder with associated myeloid malignancy (FPD/AML, associated with mutations in RUNX1), thrombocytopenia associated with a mutation in Filamin-A, or thrombocytopenia associated with a mutation in GATA-1). In some embodiments, the thrombocytopenia is immune thrombocytopenia. In some embodiments, the methods described herein increase platelet levels, increase or induce megakaryocyte differentiation and/or maturation, promote or increase platelet formation or production, reduce the accumulation of platelet progenitor cells, and/or improve blood clotting, reduce bleeding events, and/or reduce bleeding in the skin (petechiae or bruising) compared to measurements obtained prior to treatment or compared to measurements typically observed in untreated subjects having the same disease or condition. In some embodiments, the subject is identified as having thrombocytopenia prior to treatment with an ActRII chimera described herein. In some embodiments, the method includes a step of identifying the subject as having thrombocytopenia (e.g., by evaluating platelet levels) prior to treatment with an ActRII chimera described herein. The method can further include evaluating platelet levels after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing neutropenia by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, a subject having or at risk of developing low neutrophil levels (e.g., low neutrophil cell counts) has or is at risk of developing neutropenia. In some embodiments, the neutropenia is associated with a bone marrow defect, a myelodysplastic syndrome, bone marrow transplantation, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer (e.g., leukemia), a vitamin deficiency (e.g., B-12 deficiency or folate deficiency), an enlarged spleen, an autoimmune disease (e.g., granulomatosis with polyangiitis, lupus (e.g., SLE), Evans syndrome, Felty syndrome, Crohn's disease, or rheumatoid arthritis), a viral infection (e.g., chickenpox, Epstein-Barr, Hepatitis A, Hepatitis B, Hepatitis C, HIV/AIDS, cytomegalovirus, Dengue fever, or measles), a bacterial infection (e.g., tuberculosis, salmonella infection, or sepsis), cancer treatment (e.g., chemotherapy or radiation therapy), treatment with other medications (e.g., a medication used to treat overactive thyroid, such as methimazole and propylthiouracil; an antibiotic, such as vancomycin, penicillin G, trimethoprim, and oxacillin; an antiviral drug, such as ganciclovir and valganciclovir; an anti-inflammatory medication for ulcerative colitis or rheumatoid arthritis, such as sulfasalazine; a drug used to treat irregular heart rhythms, such as quinidine and procainamide; an anticonvulsant, such as phenytoin and valproate; an antipsychotic, such as clozapine; or levamisole), inflammation, hematopoietic stem cell transplantation, or contraindication to transfusion (e.g., in a patient of advanced age, a patient with allo- or auto-antibodies, a pediatric patient, a patient with cardiopulmonary disease, a patient who objects to transfusion for religious reasons (e.g., some Jehovah's Witnesses)). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)) myelodysplastic syndrome associated with isolated del chromosome abnormality (MDS with isolated del (5q)), myelodysplastic syndrome with excess blasts (MDS-EB; which includes myelodysplastic syndrome with excess blasts-type 1 (MDS-EB-1) and myelodysplastic syndrome with excess blasts-type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk MDS as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations). In some embodiments, the MDS is associated with a defect in early-stage hematopoiesis (e.g., commitment or early differentiation). In some embodiments, the MDS is associated with elevated endogenous erythropoietin levels. In some embodiments, the myelodysplastic syndrome is associated with hypocellular bone marrow (e.g., a subject with MDS has hypocellular bone marrow). The subject may have a low transfusion burden or a high transfusion burden. In some embodiments, the subject has a low transfusion burden and received 1-3 RBC units in the eight weeks prior to treatment with an ActRII chimera described herein. In some embodiments, the subject has a low transfusion burden and did not receive a transfusion (received 0 RBC units) in the eight weeks prior to treatment with an ActRII chimera described herein. In some embodiments, the subject does not respond well to erythropoietin (EPO) or is susceptible to adverse effects of EPO (e.g., hypertension, headaches, vascular thrombosis, influenza-like syndrome, obstruction of shunts, and myocardial infarction). The compositions and methods described herein can also be used to treat subjects that do not respond to an erythroid maturation agent. In some embodiments, the subject has previously been treated with an ESA. In some embodiments, the subject has not previously been treated with an ESA. In some embodiments, the neutropenia is chronic idiopathic neutropenia. In some embodiments, the neutropenia is familial neutropenia (also referred to as inherited neutropenia, e.g., cyclic neutropenia, chronic benign neutropenia, or severe congenital neutropenia (SCN), which may be associated with mutations in the genes ELANE (associated with SCN1), HAX1 (associated with SCN3), G6PC3 (associated with SCN4), GFI1 (associated with SCN2), CSF3R, WAS (associated with X-linked neutropenia/X-linked SCN), CXCR4, VPS45A (associated with SCN5), or JAGN1). In some embodiments, the methods described herein increase neutrophil levels, increase or induce neutrophil formation or production, and/or increase or induce the differentiation and/or maturation of progenitor cells (e.g., myeloid progenitors, myeloblasts, or myelocytes) into neutrophils compared to measurements obtained prior to treatment or compared to measurements typically observed in untreated subjects having the same disease or condition. In some embodiments, the methods described herein reduce the susceptibility of the subject to infection. In some embodiments, the subject is identified as having neutropenia prior to treatment with an ActRII chimera described herein. In some embodiments, the method includes a step of identifying the subject as having neutropenia (e.g., by evaluating neutrophil levels) prior to treatment with an ActRII chimera described herein. The method can further include evaluating neutrophil levels after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing fibrosis by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, the subject has or is at risk of developing fibrosis. In some embodiments, the fibrosis is fibrosis is chemotherapeutic drug-induced fibrosis, radiation-induced fibrosis, pulmonary fibrosis (e.g., cystic fibrosis, idiopathic fibrosis, or fibrosis related to tuberculosis, pneumonia, or coal dust), hepatic fibrosis (e.g., cirrhosis, biliary atresia), renal fibrosis (e.g., fibrosis related to chronic kidney disease), corneal fibrosis, heart fibrosis (e.g., endomyocardial fibrosis, or fibrosis related to myocardial infarction), bone marrow fibrosis, myelofibrosis, mediastinal fibrosis, retroperitoneal fibrosis, arthrofibrosis, osteoarticular fibrosis, tissue fibrosis (e.g., fibrosis affecting muscle tissue, skin epidermis, skin dermis, tendon, cartilage, pancreatic tissue, uterine tissue, neural tissue, testis, ovary, adrenal gland, artery, vein, bone marrow, colon, small intestine, large intestine, biliary tract, or gut), a tumor stroma, a desmoplastic tumor, a surgical adhesion, a hypertrophic scar, or a keloid. In some embodiments, the fibrosis is associated with a wound, a burn, hepatitis B or C infection, fatty liver disease, Schistosoma infection, kidney disease (e.g., chronic kidney disease), heart disease, macular degeneration, retinal or vitreal retinopathy, Crohn's disease, systemic or local scleroderma, atherosclerosis, or restenosis. In some embodiments, the subject is at risk of developing fibrosis related to cancer treatment (chemotherapy or radiation), disease or infection (e.g., tuberculosis, pneumonia, myocardial infarction, hepatitis B or C infection, fatty liver disease, Schistosoma infection, kidney disease (e.g., chronic kidney disease), heart disease, macular degeneration, retinal or vitreal retinopathy, Crohn's disease, systemic or local scleroderma, atherosclerosis, restenosis), surgery, a wound, or a burn. In some embodiments, the methods described herein reduce fibrosis compared to measurements obtained prior to treatment or compared to fibrosis in untreated subjects. In some embodiments, the methods described herein prevent the development of fibrosis or reduce the risk of developing fibrosis (e.g., reduce the risk of developing fibrosis compared to the development of fibrosis in untreated subjects). In some embodiments, the methods described herein slow or stop the progression of fibrosis (e.g., slow the progression of fibrosis compared to progression prior to treatment or compared to progression without treatment or in an untreated subject). In some embodiments, the methods described herein reduce the frequency or severity of one or more symptom of fibrosis. In some embodiments, the methods described herein improve organ or tissue function (e.g., the function of the organ or tissue having fibrosis) compared to organ or tissue function prior to treatment. Tissue and organ function can be assessed using any standard clinical test commonly used to evaluate tissue and organ function. In some embodiments, the subject is identified as having fibrosis prior to treatment with an ActRII chimera described herein. In some embodiments, the method includes a step of identifying the subject as having fibrosis (e.g., using imaging to visualize scar formation) prior to treatment with an ActRII chimera described herein. The method can further include evaluating fibrosis after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing PH (e.g., PAH, venous PH, hypoxic PH, thromboembolic PH, or miscellaneous PH) by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In any of the methods described herein, the subject may have or be at risk of developing PH. In some embodiments, the PH is PAH. In some embodiments, the PAH is idiopathic PAH. In some embodiments, the PAH is heritable PAH. In some embodiments, the PAH is PAH related to (e.g., caused by or associated with) HIV infection, schistosomiasis, portal hypertension, pulmonary veno-occlusive disease, pulmonary capillary hemangiomatosis, cirrhosis of the liver, a congenital heart abnormality, a connective tissue/autoimmune disorder (e.g., scleroderma or lupus), or drug use or abuse (e.g., methamphetamine or cocaine use). In some embodiments, the PH is venous PH. In some embodiments, the venous PH is venous PH related to (e.g., caused by or associated with) left ventricular systolic dysfunction, left ventricular diastolic dysfunction, valvular heart disease, congenital cardiomyopathy, or congenital/acquired pulmonary venous stenosis. In some embodiments, the PH is hypoxic PH. In some embodiments, the hypoxic PH is hypoxic PH related to (e.g., caused by or associated with) chronic obstructive pulmonary disease (e.g., emphysema), interstitial lung disease, sleep-disordered breathing (e.g., sleep apnea), lung disease (e.g., pulmonary fibrosis), an alveolar hypoventilation disorder, chronic exposure to high altitude, or a developmental abnormality. In some embodiments, the PH is thromboembolic PH. In some embodiments, the thromboembolic PH is thromboembolic PH related to (e.g., caused by or associated with) chronic thromboembolic pulmonary hypertension, or another pulmonary artery obstruction (e.g., a pulmonary embolism, angiosarcoma, arteritis, congenital pulmonary artery stenosis, or a parasitic infection). In some embodiments, the PH is miscellaneous PH. In some embodiments, the miscellaneous PH is miscellaneous PH related to (e.g., caused by or associated with) a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or a thyroid disease), pulmonary tumoral thrombotic microangiopathy, fibrosing mediastinitis, chronic kidney failure, or segmental pulmonary hypertension. In some embodiments, the methods described herein reduce the symptoms (e.g., reduce the severity or frequency of symptoms, such as shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting) of PH compared to the frequency or severity of symptoms prior to treatment. In some embodiments, the methods described herein prevent the development of PH or reduce the risk of developing PH (e.g., reduce the risk of developing PH compared to the development of PH in untreated subjects). In some embodiments, the methods described herein slow or stop the progression of PH (e.g., slow the progression of PH compared to progression prior to treatment or compared to progression without treatment or in an untreated subject). In some embodiments, the methods described herein reduce pulmonary vascular remodeling or vascular remodeling in the heart of a subject (e.g., the initiation or progression of vascular remodeling in the heart or lungs) compared to vascular remodeling prior to treatment or compared to vascular remodeling in an untreated subject. In some embodiments, the methods described herein reduce right ventricular hypertrophy (e.g., reduce right ventricular hypertrophy or the progression of right ventricular hypertrophy) compared to right ventricular hypertrophy prior to treatment or compared to right ventricular hypertrophy in an untreated subject. In some embodiments, the methods described herein reduce PH-associated bone loss (e.g., reduce PAH-associated bone loss, such as preventing or reducing the reduction in bone mineral density that occurs in subjects with PAH) compared to bone loss prior to treatment or compared to bone loss in an untreated subject. In some embodiments, the methods described herein reduce pulmonary arterial muscularization and/or pulmonary arterial wall thickening compared to pulmonary arterial muscularization and/or pulmonary arterial wall thickening prior to treatment or compared to pulmonary arterial muscularization and/or pulmonary arterial wall thickening in an untreated subject. In some embodiments, the methods described herein reduce right ventricular compensation compared to right ventricular compensation prior to treatment or compared to right ventricular compensation in an untreated subject. Symptoms of PH can be evaluated before and after treatment using standard clinical tests. Commonly used tests for evaluating PH include electrocardiograms, pulmonary function tests, echocardiograms, right heart catheterization, computed tomography scan, measurement of pulmonary vascular resistance, and the 6-minute walk test. In some embodiments, the methods described herein reduce pulmonary vascular resistance (e.g., result in a reduction in pulmonary vascular resistance compared to pulmonary vascular resistance prior to treatment). In some embodiments, the methods described herein improve performance in the 6-minute walk test compared to performance in the 6-minute walk test prior to treatment. In some embodiments, the subject is identified as having PH prior to treatment with an ActRII chimera described herein. In some embodiments, the method includes a step of identifying the subject as having PH (e.g., by evaluating symptoms of PH) prior to treatment with an ActRII chimera described herein. The method can further include evaluating PH symptoms after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing a metabolic disease (e.g., obesity, Type 1 diabetes, or Type 2 diabetes) by administering to the subject an effective amount of a polypeptide described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126). In some embodiments, the subject may have a disease that results in obesity. In some embodiments, the polypeptides described herein may be administered to a subject to prevent the development of obesity (e.g., in a subject at risk of developing obesity, e.g., a subject who is overweight, who has a family history of obesity, or who has another medical condition or risk factor linked to increased risk of obesity (e.g., advanced age, or treatment with a medication associated with the development of obesity, such as a glucocorticoid (e.g., a corticosteroid, such as prednisone), a selective serotonin reuptake inhibitor (SSRI, e.g., paroxetine, mirtazapine, fluoxetine, escitalopram, sertraline), a tricyclic antidepressant (e.g., amitriptyline), a mood stabilizer (e.g., valproic acid, lithium), an antipsychotic (e.g., olanzapine, chlorpromazine, clozapine), or a diabetes medication (e.g., insulin, chlorpropamide)) and/or to treat a subject already diagnosed with obesity. In some embodiments, the subject has age-related obesity or metabolic disease. In some embodiments, the subject has treatment-related obesity or metabolic disease. Administration of an ActRII chimera described herein may reduce bodyweight by decreasing the amount of body fat. In some embodiments, the ActRII chimera decreases the amount of body fat while maintaining or increasing the amount of lean mass.

In some embodiments, the polypeptides described herein may be administered to a subject to prevent the development of diabetes (e.g., Type 1 or Type 2 diabetes, e.g., in a subject at risk of developing diabetes associated with advanced age or treatment with a medication associated with the development of diabetes, such as a glucocorticoid (e.g., a corticosteroid, e.g., glucocorticoid-induced diabetes mellitus), an SSRI, a serotonin-norepinephrine reuptake inhibitor (SNRI), a mood stabilizer (e.g., lithium or valproic acid), and an antipsychotic (e.g., olanzapine and clozapine)) and/or to treat a subject already diagnosed with diabetes. In some embodiments, the subject has age-related diabetes or metabolic disease. In some embodiments, the subject has treatment-related diabetes or metabolic disease. Subjects who are likely to develop diabetes, e.g., subjects with a genetic predisposition to diabetes, a family history of diabetes, prediabetes, an autoimmune disease associated with diabetes, another metabolic disease, subjects of advanced age, or subjects treated with a medication associated with the development of diabetes may be administered the polypeptides described herein (e.g., a polypeptide including an ActRII chimera described herein) prophylactically, such that the extracellular ActRII chimeras may maintain the normal function and health of B-cells and/or prevent or delay autoimmune inflammatory damage to B-cells. In other embodiments, the polypeptides described herein (e.g., a polypeptide including an ActRII chimera described herein) may be administered to individuals before diagnosis with diabetes (e.g., Type 1 and Type 2 diabetes) or the development of clinical symptoms of diabetes, e.g., high blood glucose level, high fasting insulin level, insulin resistance, polyuria, polydipsia, and polyphagia. In some embodiments, the extracellular ActRII chimeras may be administered to patients prior to the patients needing insulin. In some embodiments, the administration of extracellular ActRII chimeras may delay, reduce, or eliminate the need for insulin treatment in diabetic patients. For example, administration of the extracellular ActRII chimeras of the invention to a subject may help to increase the rate of glucose clearance from the blood.

In some embodiments, the methods described herein reduce body fat (e.g., reduce the amount of subcutaneous, visceral, and/or hepatic fat, reduce adiposity, reduce the weights of epididymal and perirenal fat pads, or reduce body fat percentage). In some embodiments, the methods described herein reduce body weight or reduce body weight gain (e.g., reduce the percentage of body weight gain). In some embodiments, the methods described herein reduce the proliferation of adipose cells. In some embodiments, the methods described herein reduce LDL. In some embodiments, the methods described herein reduce triglycerides. In some embodiments, the methods described herein improve the serum lipid profile of the subject. In some embodiments, the methods described herein reduce body fat and increase muscle mass. In some embodiments, the methods described herein reduce blood glucose levels (e.g., fasting glucose levels) or and/or increase glucose clearance. In some embodiments, the methods described herein reduce fasting insulin levels and/or improve insulin sensitivity (e.g., reduce insulin resistance). In some embodiments, the methods described herein regulate insulin biosynthesis and/or secretion from B-cells. These outcomes can be assessed by comparing measurements obtained after treatment to measurements taken prior to treatment. In some embodiments, the methods described herein do not affect the appetite for food intake. The polypeptides described herein (e.g., a polypeptide including an extracellular ActRII chimera described herein) may decrease body fat, decrease body weight, or increase insulin sensitivity and/or glucose clearance by increasing muscle mass. In some embodiments, the subject is identified as having a metabolic disease prior to treatment with an ActRII chimera described herein. In some embodiments, the method includes a step of identifying the subject as having a metabolic disease (e.g., by evaluating body weight, body fat, glucose clearance, or insulin sensitivity) prior to treatment with an ActRII chimera described herein. The method can further include evaluating body fat (e.g., amount of body fat or body fat percentage), body weight or body weight gain, fasting insulin levels, glucose clearance, serum lipid profile, or insulin sensitivity after administration of an ActRII chimera described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

In some embodiments, the polypeptides described herein (e.g., a polypeptide including an extracellular ActRII chimera described herein, e.g., an extracellular ActRII chimera of Table 1 or Table 2, e.g., an extracellular ActRII chimera of any one of SEQ ID NOs: 96-126, e.g., an effective amount of an ActRII chimera) can be administered to increase EPO levels (e.g., serum EPO levels) and/or EPO receptor levels (e.g., EPO receptor levels in bone marrow cells) in a subject in need thereof (e.g., a subject with low serum EPO). The invention also includes methods of treating a subject having or at risk of developing (e.g., treating, delaying the development of, and/or preventing) a disease or condition that can be treated with EPO or an ESA (e.g., a disease or condition that can be treated by increasing EPO or EPO receptor levels) by administering to the subject an effective amount of a polypeptide described herein (e.g., a polypeptide including an extracellular ActRII chimera described herein (e.g., an effective amount of an ActRII chimera)). Diseases and conditions that can be treated by increasing EPO or EPO receptor levels include end-stage renal disease, renal insufficiency, polycythemia, anemia due to dialysis, early anemia of prematurity, iron overload (e.g., hemochromatosis), pregnancy, a menstrual disorder, space flight, ischemia (CNS ischemia, liver ischemia, renal ischemia, or cardiac ischemia), ulcers, burns, wounds (e.g., chronic wounds), ischemia-reperfusion injury (e.g., ischemia-reperfusion injury associated with surgery or organ transplantation), an ischemic disorder or condition (e.g., myocardial infarction, ischemic stroke, occlusive arterial disease, chronic venous insufficiency, pulmonary embolism, circulatory shock, such as hemorrhagic, septic, or cardiogenic shock, acute respiratory failure, chronic heart failure, atherosclerosis, cardiac cirrhosis, macular degeneration, sleep apnea, Raynaud's disease, systemic sclerosis, nonbacterial thrombotic endocarditis, angina pectoris, transient ischemic attacks, chronic alcoholic liver disease, or ischemia resulting from general anesthesia), hypoxia (e.g., perinatal hypoxia or a hypoxic condition or disorder such as a pulmonary disorder (e.g., hypoxic hypoxia, such as COPD), severe pneumonia, pulmonary edema, hyaline membrane disease, liver or renal disease, cancer or other chronic illness, and altitude sickness), and aging. A polypeptide described herein can also be used to treat a subject receiving kidney dialysis, to treat a subject who has recently received a stem cell transplant, to increase red blood cell count in a subject prior to surgery, or as a pretreatment or further treatment for a tissue or organ to be transplanted (such as for treatment of the tissue or organ before (e.g., directly before), during, or directly after transplantation).

Given that EPO has been found to stimulate the mobilization, proliferation, migration, and differentiation of endothelial progenitor cells, the polypeptides described herein can also be used to treat a disease associated with dysfunction of endothelial progenitor cells. Such diseases include heart failure, angina pectoris, endotheliosis (e.g., reticuloendotheliosis), age-related cardiovascular disorder, coronary heart disease, atherosclerosis, myocardial ischemia, hypercholesterolemia, ischemic disorders of the extremities, Raynaud's disease, preeclampsia, pregnancy-induced hypertension, endothelium-mediated chronic inflammatory disorders (e.g., inflammation of the vessels), wound healing, and chronic or acute renal failure (also referred to as chronic kidney disease and acute kidney failure, respectively). Since EPO has been shown to have a mitogenic and chemotactic effect on vascular endothelial cells, the ActRII chimera polypeptides can also be used to promote the growth of new blood vessels (vasculogenesis) and/or the replacement of damaged vascular regions through local formation of new blood vessels, such as collateral coronary blood vessels (e.g., those that may occur after myocardial infarction), for granulation tissue formation (e.g. in damaged tissue, wounds, and ulcers), for trauma treatment, for post-vascular graft treatment, and for production of vascular prostheses such as heart valves.

EPO has also been found to have anti-inflammatory and neuroprotective effects. Therefore, the polypeptides described herein can also be used to treat a neurological disorder and/or an inflammatory brain disease, such as a demyelinating disease (e.g., multiple sclerosis, neuromyelitis optica, acute disseminated encephalomyelitis, transverse myelitis), epilepsy, spinal cord injury (e.g., an acute spinal cord injury), a complication following traumatic brain injury (e.g., to treat a symptom of the traumatic brain injury, such as hypotension, hypoxemia, brain swelling, headache, neck pain, difficulty remembering, difficulty concentrating, difficulty making decisions, fatigue, a mood change, nausea, photophobia, blurred vision, ear ringing, a loss of sense of taste, a loss of sense of smell, a seizure, coma, muscle weakness, paralysis, or a progressive decline in neurologic function), a chronic inflammatory brain disease (e.g., a neurodegenerative disease, such as Alzheimer's disease (AD), Parkinson's disease (PD), Huntington's disease, amyotrophic lateral sclerosis (ALS), or age-related macular degeneration (AMD)), or a neurological disorder associated with surgery, such as thoracoabdominal aortic surgery, in addition to diseases or conditions that have an inflammatory or autoimmune component, such as acute cerebrovascular injury, acute brain injury, acute cardiovascular injury, arthritis, an autoimmune disease, a stroke, a neurological injury, and immune-mediated inflammation. The polypeptide may treat the neurological disorder or inflammatory brain disease by reducing infiltration of mononuclear cells into the brain of the subject, improving a neurological deficit, and/or reducing axonal damage and/or neuronal and/or glial cell death in at least one region of the brain of the subject affected, directly or indirectly, by the disease, disorder, or condition.

Gastrointestinal dysmotility can also be treated using EPO. Accordingly, the polypeptides described herein may be used to treat gastrointestinal dysmotility due to intestinal injury, abdominal trauma, an intestinal inflammatory condition (e.g., an inflammatory bowel disease (IBD), such as Crohn's Disease and Ulcerative Colitis), an intestinal infection (e.g., a bacterial infection, such as an infection that leads to sepsis and bacteremia and localized infections such as peritonitis and ascites), slow transit constipation (e.g., chronic constipation, idiopathic constipation, constipation due to post-operative ileus, or constipation caused by opiate use), post-operative ileus, a neurodegenerative injury, a neurotraumatic injury, a congenital problem (e.g., Gastroschisis, omphalocele, aganglionic megacolon, Hirschsprung's disease, chronic intestinal pseudo-obstruction, small left colon syndrome, an anorectal anomaly, esophageal dysplasia and atresias, ectopic anus, a congenital hernia, internal anal sphincter achalasia), or a malnutrition-malabsorption problem (e.g., due to an intestinal injury, an abdominal trauma, an intestinal inflammatory condition, an intestinal infection, constipation (e.g., constipation caused by opiate use), post-operative ileus, a neurodegenerative injury, a neurotraumatic injury, a congenital problem, Gaucher disease, refeeding syndrome, extremely low birth weight infants, cancer cachexia, infection, cancer, spinal cord dysfunction, spinal dysraphism, bifida, tumor, central nervous system dysfunction, peripheral neuropathy, removal of part of the gastrointestinal tract, hemorrhage, liver dysfunction, celiac disease, cystic fibrosis, a muscular dystrophy, or cerebral palsy).

The polypeptides described herein can also be used to treat chronic or recurrent disease such as asthma, a viral disease or infection (e.g., HIV infection or HCV infection), hypertension, a systemic microbial infection, cancer, a disease of the endocrine system, a disease of the reproductive system, psychosis, a genetic disease, allergy, a gastrointestinal disease, arterial sclerosis, a cardiovascular disease, graft-vs-host disease, or an inflammatory disease. Polypeptides containing an ActRII chimera can also be used to enhance athletic performance, improve exercise capacity, and facilitate or enhance aerobic conditioning. Such methods can be used, e.g., by athletes to facilitate training and by soldiers to improve stamina and endurance. In some embodiments, the methods described herein are directed to affecting myostatin, activin A, activin B, and/or BMP9 signaling (e.g., reducing or inhibiting the binding of activin A, activin B, myostatin, and/or BMP9 to their endogenous receptors, e.g., ActRIIA, ActRIIB, and/or BMPRII) in a subject having a disease or condition that can be treated with EPO or an ESA. In some embodiments, the methods described herein increase EPO levels (e.g., serum EPO levels) and/or EPO receptor levels (e.g., bone marrow EPO receptor levels) compared to measurements obtained prior to treatment or compared to measurements obtained from untreated subjects or control treated subjects having the same disease or condition.

In any of the methods described herein, a polypeptide including an extracellular ActRII chimera described herein (e.g., an extracellular ActRII chimera of Table 1 or Table 2, e.g., an extracellular ActRII chimera of any one of SEQ ID NOs: 96-126) that further includes a C-terminal extension of one to six amino acids (e.g., 1, 2, 3, 4, 5, 6 or more amino acids from extracellular ActRIIA or ActRIIB) may be used as the therapeutic protein. In any of the methods described herein, a dimer (e.g., homodimer or heterodimer) formed by the interaction of two Fc domain monomers that are each fused to a polypeptide including an extracellular ActRII chimera described herein (e.g., an extracellular ActRII chimera of Table 1 or Table 2, e.g., an extracellular ActRII chimera of any one of SEQ ID NOs: 96-126) may be used as the therapeutic protein. In any of the methods described herein, a polypeptide including an extracellular ActRII chimera described herein (e.g., an extracellular ActRII chimera of Table 1 or Table 2, e.g., an extracellular ActRII chimera of any one of SEQ ID NOs: 96-126) fused to a moiety (e.g., an Fc domain, an albumin-binding peptide, a fibronectin domain, or a serum albumin) may be used as the therapeutic protein. Nucleic acids encoding the polypeptides described herein, or vectors containing said nucleic acids can also be administered according to any of the methods described herein. In any of the methods described herein, the polypeptide, nucleic acid, or vector can be administered as part of a pharmaceutical composition.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Evaluation of ActRII Chimeras Using a Luciferase Gene Reporter Assay

In this experiment, chimera-Fc proteins were assayed for their ability to inhibit BMP-9, activin A, activin B, and GDF-11 signaling. Briefly, the ligands (BMP-9, activin A, activin B, or GDF-11) were incubated with cells expressing a luciferase reporter, which activates the downstream signaling that results in luciferase expression. When the ligands are pre-incubated with a functional inhibitor (e.g., a chimera-Fc protein) before being added to the cells, a loss of luciferase signal corresponds to the extent of ligand inhibition.

Two stably transfected luciferase reporter systems were used to assess cellular inhibition of signaling by BMP-9, activin A, activin B, and GDF-11. C2C12 cells containing a BMP-responsive BRE-Luciferase construct (produced using protocol from Zilberberg, 2007) were used to assess inhibition of BMP-9 signaling, and HEK293 cells containing a Smad binding element SBE-Luciferase (BPS Bioscience) were used to assess inhibition of activin A, activin B, and GDF-11 signaling. Cells were plated on 96-well plates in DMEM supplemented with 10% FBS and placed in an incubator overnight to acclimate to the plate surface. A dilution series spanning between 7.8 ng/ml to 200 μg/mL of each chimera-Fc was made in 0.1% DMEM at concentrations spanning the IC₅₀ and incubated with Activin A (2 nM), Activin B (2 nM), GDF-11 (4 nM), or BMP-9 (0.4 nM) for 60 minutes at 37° C. Wells containing only the ligand and no chimera-Fc served as the positive control against which inhibition was calculated. Media on the plates was aspirated and the chimera-Fc/ligand mixtures were added to the plates as media replacement. The remaining wells were used for replicates of positive controls and background. The plates were incubated overnight at 37° C., Promega Steady Glo was added to the plate according to the kit instructions, and the ODs of the wells were read on a Perkin Elmer Ensight Multimode Microplate Reader. Percent inhibition was calculated for each well by comparing the luciferase signal observed to the signal generated by the positive control. By testing a series of ActRII concentrations ranging from no inhibition to complete inhibition, the IC₅₀ concentrations were calculated using GraphPad Prism software in a 4-parameter nonlinear regression model. The cell-based assays demonstrate the ability of the chimeras to inhibit signaling at the endogenous cell surface receptors (Tables 3 and 4).

TABLE 3
Reporter cell assay results for ActRIIA-based variants

| | Activin A | Activin B | GDF11 | BMP9 |
|---|---|---|---|---|
| Construct | (ng/ml) | (ng/ml) | (ng/ml) | (μg/mL) |
| ActRIIA-A1 | 640 | 210 | 240 | 50 |
| (SEQ ID NO: 104) | | | | |
| ActRIIA-A2 | 380 | 94 | 170 | 86 |
| (SEQ ID NO: 105) | | | | |
| ActRIIA-A3 | 630 | 240 | 190 | 12 |
| (SEQ ID NO: 106) | | | | |
| ActRIIA-A5 | 300 | 73 | 230 | 4.2 |
| (SEQ ID NO: 107) | | | | |
| ActRIIA-A6 | 330 | 100 | 77 | 8.8 |
| (SEQ ID NO: 108) | | | | |
| ActRIIA-A7 | 470 | 110 | 260 | 47 |
| (SEQ ID NO: 109) | | | | |
| ActRIIA-A8 | 75 | 100 | 180 | 1.9 |
| (SEQ ID NO: 110) | | | | |
| ActRIIA-B2 | Not evaluated | | | |
| (SEQ ID NO: 96) | | | | |
| ActRIIA-B3 | 140 | 71 | 130 | >50 |
| (SEQ ID NO: 97) | | | | |
| ActRIIA-B4 | 320 | 94 | 220 | >50 |
| (SEQ ID NO: 98) | | | | |
| ActRIIA-B5 | >1000 | >500 | 810 | >50 |
| (SEQ ID NO: 99) | | | | |
| ActRIIA-B7 | 840 | 180 | 230 | 21 |
| (SEQ ID NO: 100) | | | | |
| ActRIIA-B1B4 | 750 | 130 | 260 | >50 |
| (SEQ ID NO: 102) | | | | |
| ActRIIA-B1B7 | 1400 | 250 | 390 | >50 |
| (SEQ ID NO: 101) | | | | |
| ActRIIA-B2B4 | 700 | 160 | 290 | >50 |
| (SEQ ID NO: 103) | | | | |

TABLE 4
Reporter cell assay results for ActRIIB-based variants

| | Activin A | Activin B | GDF11 | BMP9 |
|---|---|---|---|---|
| Construct | (ng/ml) | (ng/ml) | (ng/mL) | (ug/mL) |
| ActRIIB-A1 | 75 | 72 | 140 | 0.14 |
| (SEQ ID NO: 120) | | | | |
| ActRIIB-A2 | 78 | 130 | 300 | 0.082 |
| (SEQ ID NO: 121) | | | | |
| ActRIIB-A3 | 56 | 75 | 150 | 0.1 |
| (SEQ ID NO: 122) | | | | |
| ActRIIB-A5 | 57 | 72 | 130 | 0.4 |
| (SEQ ID NO: 123) | | | | |
| ActRIIB-A6 | 89 | 140 | 250 | 0.23 |
| (SEQ ID NO: 124) | | | | |
| ActRIIB-A7 | 57 | 73 | 130 | 0.061 |
| (SEQ ID NO: 125) | | | | |
| ActRIIB-A8 | 230 | 52 | 130 | 3.6 |
| (SEQ ID NO: 126) | | | | |
| ActRIIB-B2 | 140 | 170 | 270 | 0.3 |
| (SEQ ID NO: 111) | | | | |
| ActRIIB-B3 | 70 | 96 | 140 | 0.41 |
| (SEQ ID NO: 112) | | | | |
| ActRIIB-B4 | Not evaluated | | | |
| (SEQ ID NO: 113) | | | | |
| ActRIIB-B5 | 69 | 110 | 96 | 0.022 |
| (SEQ ID NO: 114) | | | | |
| ActRIIB-B7 | 67 | 120 | 150 | 0.032 |
| (SEQ ID NO: 115) | | | | |
| ActRIIB-B1B4 | 350 | 340 | 770 | 0.39 |
| (SEQ ID NO: 117) | | | | |
| ActRIIB-B1B7 | 94 | 180 | 140 | 0.067 |
| (SEQ ID NO: 116) | | | | |
| ActRIIB-B2B4 | Not evaluated | | | |
| (SEQ ID NO: 118) | | | | |
| ActRIIB- | 130 | 86 | 140 | 0.36 |
| B1B2B3B4 | | | | |
| (SEQ ID NO: 119) | | | | |

### Example 2—Evaluation of ActRII Chimera Binding Affinity

The Cytiva Biacore 8k was used to measure the kinetics of the interactions between the ActRII- and chimera-Fc proteins and Activin A/Activin B/growth differentiation factor 8 (GDF-8)/GDF-11/BMP-9/BMP-10. Series S CM4 Sensor Chips were immobilized with anti-human capture antibody using the reagents and protocol in the Biacore Human Antibody Capture Kit (GE Life Sciences). Briefly, anti-human IgG was diluted to 25 μg/mL in immobilization buffer. The carboxylated surface of the sensor was activated by injecting a mixture of EDC and NHS. The anti-human IgG was injected into the activated sensor chip flow cells at 10 μL/min for a total of 7 minutes until the chip reached an immobilization level of 3000 resonance units (RU). Ethanolamine was injected to deactivate the sensor surfaces. The chip has 8 flow cells to be used for binding analysis, each with an in-line reference surface to subtract instrument noise and non-specific binding to the sensor chip. The ActRII- and chimera-Fc proteins were captured in their own flow cell to allow for a maximum analyte binding response of 20-30 RU. This was done by injecting 10-20 μL of the ActRII- and chimera-Fc proteins into the flow cells at a concentration of 2 μg/mL until 60-90 RU was captured. Ligand at a single concentration was injected at 35 L/min for 60 seconds and then allowed to dissociate under buffer flow for no less than 5 minutes. After the cycle completed, captured protein was stripped from the chip with an injection of 3 M magnesium chloride, the regeneration buffer provided with the Biacore Human Antibody Capture Kit. Each ligand was injected in a concentration series, including a buffer only 0 nM concentration to serve as a double reference for subtracting out instrument noise. The data were analyzed using the Biacore Insight Evaluation Software.

TABLE 5
K_{D}(M) for ActRIIA-based constructs

| Construct | Activin A | Activin B | GDF8 | GDF11 | BMP9 | BMP10 |
|---|---|---|---|---|---|---|
| ActRIIA-A1 | 7.03E−11 | 1.99E−11 | 2.03E−10 | <1E−12 | 6.20E−08 | 6.63E−11 |
| (SEQ ID NO: 104) | | | | | | |
| ActRIIA-A2 | 5.79E−11 | 1.21E−10 | 8.37E−11 | <1E−12 | 5.26E−08 | 1.88E−11 |
| (SEQ ID NO: 105) | | | | | | |
| ActRIIA-A3 | 3.57E−11 | 7.55E−11 | 9.77E−11 | <1E−12 | 1.80E−08 | 2.89E−11 |
| (SEQ ID NO: 106) | | | | | | |
| ActRIIA-A5 | 4.80E−11 | 1.74E−11 | 1.22E−10 | 6.65E−12 | 2.55E−09 | 1.26E−10 |
| (SEQ ID NO: 107) | | | | | | |
| ActRIIA-A6 | 3.20E−11 | 5.58E−11 | 6.82E−11 | <1E−12 | 4.97E−08 | 3.47E−10 |
| (SEQ ID NO: 108) | | | | | | |
| ActRIIA-A7 | 2.08E−10 | 5.64E−12 | 1.26E−10 | <1E−12 | 5.30E−08 | 3.15E−11 |
| (SEQ ID NO: 109) | | | | | | |
| ActRIIA-A8 | 4.00E−11 | <1E−12 | 2.29E−10 | <1E−12 | 8.10E−09 | 3.97E−11 |
| (SEQ ID NO: 110) | | | | | | |
| ActRIIA-B2 | Not evaluated | | | | | |
| (SEQ ID NO: 96) | | | | | | |
| ActRIIA-B3 | 2.56E−11 | <1E−12 | 2.48E−11 | <1E−12 | 1.36E−08 | 1.22E−11 |
| (SEQ ID NO: 97) | | | | | | |
| ActRIIA-B4 | 1.86E−11 | <1E−12 | 1.15E−10 | <1E−12 | 1.36E−08 | 4.60E−11 |
| (SEQ ID NO: 98) | | | | | | |
| ActRIIA-B5 | 6.54E−11 | 2.70E−11 | 6.82E−10 | 5.69E−11 | no binding | 1.17E−09 |
| (SEQ ID NO: 99) | | | | | | |
| ActRIIA-B7 | 2.40E−11 | 8.89E−12 | 1.21E−11 | <1E−12 | 5.89E−08 | 1.41E−10 |
| (SEQ ID NO: 100) | | | | | | |
| ActRIIA-B1B4 | 2.40E−11 | 5.31E−12 | 1.25E−10 | 3.43E−09 | 3.29E−08 | 2.27E−10 |
| (SEQ ID NO: 102) | | | | | | |
| ActRIIA-B1B7 | 2.17E−11 | 2.49E−11 | 8.98E−11 | <1E−12 | 7.72E−08 | 2.00E−10 |
| (SEQ ID NO: 101) | | | | | | |
| ActRIIA-B2B4 | 1.60E−11 | <1E−12 | 6.72E−11 | <1E−12 | 1.31E−08 | 6.05E−11 |
| (SEQ ID NO: 103) | | | | | | |
| ActRIIA | 1.60E−10 | 6.60E−11 | 2.22E−10 | <1E−12 | 5.91E−08 | 1.05E−10 |
| (SEQ ID NO: 30) | | | | | | |

TABLE 6
K_{D}(M) for ActRIIB-based constructs

| Construct | Activin A | Activin B | GDF8 | GDF11 | BMP9 | BMP10 |
|---|---|---|---|---|---|---|
| ActRIIB-A1 | 4.48E−11 | <1E−12 | 1.82E−11 | <1E−12 | 1.51E−11 | 1.75E−11 |
| (SEQ ID NO: 120) | | | | | | |
| ActRIIB-A2 | 3.47E−11 | 8.78E−12 | 3.93E−11 | <1E−12 | 1.66E−11 | 2.98E−11 |
| (SEQ ID NO: 121) | | | | | | |
| ActRIIB-A3 | 1.65E−11 | <1E−12 | <1E−12 | <1E−12 | 1.23E−11 | 1.59E−11 |
| (SEQ ID NO: 122) | | | | | | |
| ActRIIB-A5 | 2.28E−11 | 4.91E−12 | 1.65E−11 | 5.66E−12 | 1.43E−10 | 1.52E−11 |
| (SEQ ID NO: 123) | | | | | | |
| ActRIIB-A6 | 1.04E−11 | 1E−12 | 2.88E−11 | <1E−12 | 9.62E−12 | 9.69E−12 |
| (SEQ ID NO: 124) | | | | | | |
| ActRIIB-A7 | 1.69E−11 | <1E−12 | <1E−12 | <1E−12 | 1.91E−11 | 8.72E−12 |
| (SEQ ID NO: 125) | | | | | | |
| ActRIIB-A8 | 4.42E−11 | <1E−12 | 4.23E−12 | <1E−12 | 7.40E−11 | 2.92E−11 |
| (SEQ ID NO: 126) | | | | | | |
| ActRIIB-B2 | <1E−12 | 1.16E−11 | <1E−12 | <1E−12 | 3.27E−12 | 2.05E−11 |
| (SEQ ID NO: 111) | | | | | | |
| ActRIIB-B3 | 1.52E−12 | 5.81E−12 | 1.04E−11 | 1.17E−12 | 3.61E−10 | 1.75E−10 |
| (SEQ ID NO: 112) | | | | | | |
| ActRIIB-B4 | Not evaluated | | | | | |
| (SEQ ID NO: 113) | | | | | | |
| ActRIIB-B5 | 3.27E−12 | 1.58E−11 | 1.02E−11 | <1E−12 | 6.32E−11 | 4.14E−11 |
| (SEQ ID NO: 114) | | | | | | |
| ActRIIB-B7 | <1E−12 | <1E−12 | 3.46E−12 | <1E−12 | 2.02E−11 | <1E−12 |
| (SEQ ID NO: 115) | | | | | | |
| ActRIIB-B1B4 | 1.08E−11 | 7.02E−11 | 7.18E−11 | 2.92E−11 | 3.98E−11 | 7.61E−11 |
| (SEQ ID NO: 117) | | | | | | |
| ActRIIB-B1B7 | <1E−12 | <1E−12 | 3.53E−12 | <1E−12 | 1.86E−08 | 1.56E−11 |
| (SEQ ID NO: 116) | | | | | | |
| ActRIIB-B2B4 | Not evaluated | | | | | |
| (SEQ ID NO: 118) | | | | | | |
| ActRIIB-B1B2B3B4 | 6.60E−12 | 1.97E−11 | 3.54E−12 | <1E−12 | 1.08E−09 | 3.69E−10 |
| (SEQ ID NO: 119) | | | | | | |
| ActRIIB | 1.06E−11 | <1E−12 | 6.03E−12 | <1E−12 | 3.58E−11 | 1.05E−11 |
| (SEQ ID NO: 31) | | | | | | |

### Example 3—Effect of Hydrodynamic Injection of Extracellular ActRII Chimeras on Lean Mass, Muscle Mass, and Hematology

To assess the effect of extracellular ActRII chimeras on lean mass and hematology, 10-week-old wild type female C57Bl/6 mice were enrolled in the study. Pre-dosing, mice were weighed, and lean mass determined using a small rodent nuclear magnetic resonance (NMR) analyzer (Bruker, Minispec LF50). Once baseline levels were determined, each mouse was injected, via hydrodynamic tail vain injection (HDI), with vehicle (n=9) or vehicle containing an expression vector for either ActRIIA-B7 (SEQ ID NO: 100) (n=8), ActRIIA-B1β₄ (SEQ ID NO: 102) (n=9), or ActRIIA-B1β₇ (SEQ ID NO: 101) (n=9) fused to an Fc domain at a dose of 0.75 mg/kg.

### HDI Methods:

Ten-week-old female C57Bl/6 mice (n=8-9/group) received a bolus (100 ml/kg) of plasmid DNA encoding an ActRII chimera-Fc protein (ActRIIA-B7 (SEQ ID NO: 100), ActRIIA-B1B4 (SEQ ID NO: 102), or ActRIIA-B1B7 (SEQ ID NO: 101) fused to an Fc domain) or vehicle via a single hydrodynamic injection into the lateral tail vein (0.75 mg/kg). Mice were excluded if the entire injection did not flow smoothly into the vein, the injection took longer than 8 seconds, or the mice did not fully recover within 10 minutes of administration.

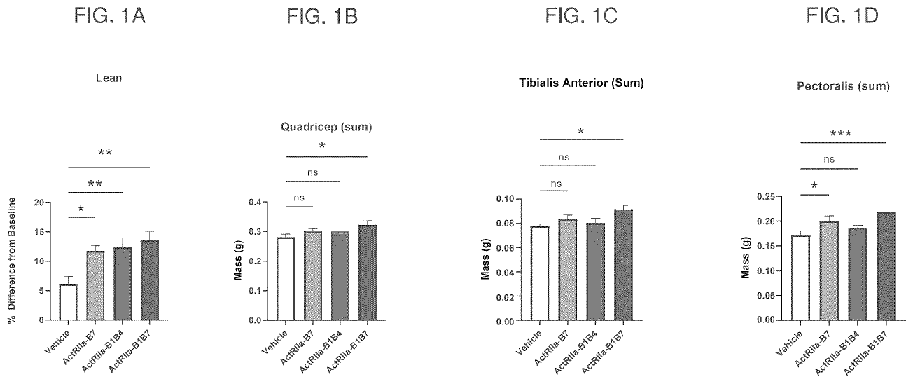

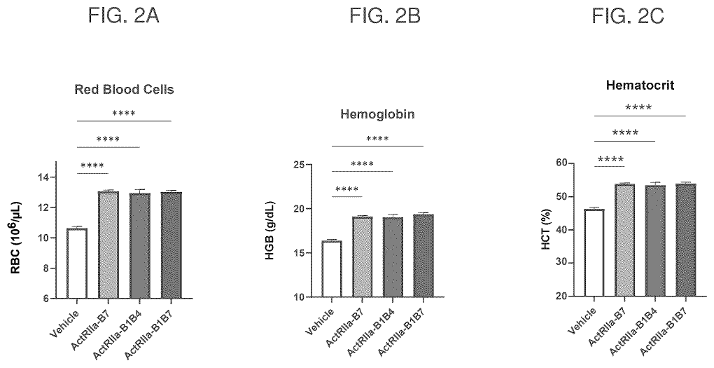

The study was terminated after 27-28 days and whole blood (EDTA), terminal body weight, and lean mass were determined. Results are shown in FIGS. 1A-1D and FIGS. 2A-2C.

### Example 4—ActRII Chimeras Increased Trabecular Bone in the Proximal Femur

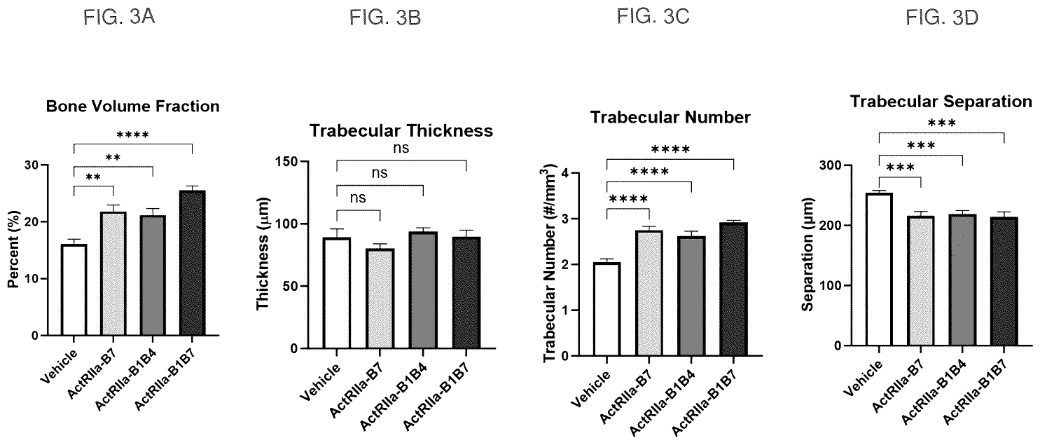

Ten-week-old female C57Bl/6 mice (n=8-9/group) received either vehicle (PBS) or 0.75 mg/kg of plasmid DNA. Plasmid DNA containing constructs expressing ActRIIA-B7 (SEQ ID NO: 100), ActRIIA-B1B4 (SEQ ID NO: 102), or ActRIIA-B1B7 (SEQ ID NO: 101) were delivered via the tail vain in a volume of 100 mls/kg. After 28 days, mice were euthanized and hindlimbs dissected and preserved by freezing in PBS-soaked gauze. For analysis, femurs were scanned using GX2 μCT (10 mm FOV, 90 KV, 88 μA, 4 minutes, Perkin Elmer). Trabecular bone at the distal femur was evaluated using Analyze 14.0 Bone Micro-architecture Analysis software (AnalyzeDirect). Data highlighting bone volume fraction (FIG. 3A), trabecular thickness (FIG. 3B), trabecular number (FIG. 3C), and trabecular separation (FIG. 3D) are shown in FIGS. 3A-3D. Data are shown as average±SEM. Stats shown, using 1-way ANOVA with a Dunnett posttest: *p≤0.05, **p<0.01, ***p<0.001, ****p<0.0001.

### Example 5—Treatment of a Muscle Disease by Administration of an Extracellular ActRII Chimera

According to the methods disclosed herein, a physician of skill in the art can treat a subject, such as a human patient, having a muscle disease (e.g., neuromuscular disease, such as a muscular dystrophy, IBM, SMA, CMT, ALS, myasthenia gravis, or multiple sclerosis; sarcopenia; or cachexia) so as to increase muscle mass or maintain or improve muscle strength (e.g., reduce muscle weakness). The method of treatment can include diagnosing or identifying a subject as a candidate for treatment based on standard clinical tests for muscle diseases (e.g., blood test, muscle biopsy, genetic test, and/or electromyogram). To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) or by local administration (e.g., injection into the muscle) to treat a muscle disease. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to increase muscle mass or maintain or improve muscle strength (e.g., reduce muscle weakness).

Following administration of the composition to a patient, a practitioner of skill in the art can monitor the patient's improvement in response to the therapy by a variety of methods. For example, a physician can monitor the patient's muscle mass, muscle strength, and motor function. A finding that the patient exhibits increased muscle mass or maintains or improves muscle strength following administration of the composition compared to test results prior to administration of the composition indicates that the patient is responding favorably to the treatment. Subsequent doses can be determined and administered as needed.

### Example 6—Treatment of a Bone Disease by Administration of an Extracellular ActRII Chimera

According to the methods disclosed herein, a physician of skill in the art can treat a subject, such as a human patient, having a bone disease (e.g., osteoporosis, osteogenesis imperfecta, or osteopenia) so as to increase bone mineral density, increase bone formation, reduce bone resorption, reduce bone loss, or reduce the risk or occurrence of bone fracture. The method of treatment can include diagnosing or identifying a subject as a candidate for treatment based on standard clinical tests for bone mineral density (e.g., dual X-ray absorptiometry). To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat a bone disease. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to increase bone mineral density, increase bone formation, reduce bone resorption, reduce bone loss, or reduce the risk or occurrence of bone fracture.

Following administration of the composition to a patient, a practitioner of skill in the art can monitor the patient's improvement in response to the therapy by a variety of methods. For example, a physician can monitor the patient's bone mineral density by performing dual X-ray absorptiometry. A finding that the patient exhibits increased bone mineral density, increased bone formation, reduced bone resorption, reduced bone loss, or a reduced risk or occurrence of bone fracture following administration of the composition compared to test results prior to administration of the composition indicates that the patient is responding favorably to the treatment. Subsequent doses can be determined and administered as needed.

### Example 7—Treatment of Anemia by Administration of an Extracellular ActRII Chimera

According to the methods disclosed herein, a physician of skill in the art can treat a subject, such as a human patient, having anemia (e.g., anemia of inflammation, anemia associated with myelofibrosis, anemia associated with a myelodysplastic syndrome, or anemia associated with chronic kidney disease) so as to increase a parameter of red cell mass, such as red blood cell count, hemoglobin levels, or hematocrit, or to increase the maturation and/or differentiation of erythroid progenitors, increase late-stage erythroid precursor maturation, increase the number of early-stage erythroid precursors and/or progenitors, promote the progression of erythroid precursors and/or progenitors through erythropoiesis, or recruit early-stage progenitors into the erythroid lineage. The method of treatment can include diagnosing or identifying a subject as a candidate for treatment based on a blood test measuring hematological parameters. To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat anemia. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to increase hemoglobin levels, increase red blood cell counts, increase hematocrit, increase the maturation and/or differentiation of erythroid progenitors, increase late-stage erythroid precursor maturation, increase the number of early-stage erythroid precursors and/or progenitors, promote the progression of erythroid precursors and/or progenitors through erythropoiesis, or recruit early-stage progenitors into the erythroid lineage. Following administration of the composition to a patient, a practitioner of skill in the art can monitor the patient's improvement in response to the therapy by a variety of methods. For example, a physician can monitor the patient's hemoglobin levels, red blood cell counts, or hematocrit by performing a blood test. A finding that the patient exhibits improved hemoglobin levels, red blood cell counts, or hematocrit following administration of the composition compared to test results prior to administration of the composition indicates that the patient is responding favorably to the treatment. Subsequent doses can be determined and administered as needed.

### Example 8—Treatment of Fibrosis by Administration of an Extracellular ActRII Chimera

According to the methods disclosed herein, a physician of skill in the art can treat a subject, such as a human patient, having fibrosis (e.g., pulmonary fibrosis, myelofibrosis, or fibrosis associated with chronic kidney disease) so as to reduce the symptoms of fibrosis or slow or stop the progression of fibrosis. The method of treatment can include diagnosing or identifying a subject as a candidate for treatment based on clinical tests for fibrosis (e.g., imaging tests, such as X-ray or CT scan). To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat fibrosis, or can be locally administered (e.g., injected) to the fibrotic tissue or organ. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to reduce the symptoms of fibrosis or slow or stop the progression of fibrosis.

Following administration of the composition to a patient, a practitioner of skill in the art can monitor the patient's improvement in response to the therapy by a variety of methods. For example, a physician can monitor the patient's fibrosis by performing imaging tests and can monitor the patient's symptoms using standard clinical tests. A finding that the patient's symptoms are reduced or that progression of the patient's fibrosis slows or stops following administration of the composition compared to test results prior to administration of the composition indicates that the patient is responding favorably to the treatment. Subsequent doses can be determined and administered as needed.

### Example 9—Treatment of Pulmonary Hypertension by Administration of an Extracellular ActRII Chimera

According to the methods disclosed herein, a physician of skill in the art can treat a subject, such as a human patient, having pulmonary hypertension (PH, e.g., PAH) so as to reduce the symptoms of PH or slow or stop the progression of PH. The method of treatment can include diagnosing or identifying a subject as a candidate for treatment based on standard clinical tests for PH (e.g., echocardiogram, electrocardiogram, chest X-ray, or right heart catheterization). To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat PH. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to reduce the symptoms of PH or slow or stop the progression of PH.

Following administration of the composition to a patient, a practitioner of skill in the art can monitor the patient's improvement in response to the therapy by a variety of methods. For example, a physician can monitor the patient's symptoms using standard clinical tests and patient self-reporting. A finding that the patient's symptoms are reduced the symptoms of PH or that progression of the patient's PH slows or stops following administration of the composition compared to test results prior to administration of the composition indicates that the patient is responding favorably to the treatment. Subsequent doses can be determined and administered as needed.

### Example 10—Treatment of a Metabolic Disease by Administration of an Extracellular ActRII Chimera

According to the methods disclosed herein, a physician of skill in the art can treat a subject, such as a human patient, having a metabolic disease (e.g., obesity) so as to reduce body weight, body fat or percent body fat, or improve the serum lipid profile of the subject. To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat obesity. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to reduce body weight, body fat or percent body fat, or improve the serum lipid profile of the subject.

Following administration of the composition to a patient, a practitioner of skill in the art can monitor the patient's improvement in response to the therapy by a variety of methods. For example, a physician can monitor the patient's symptoms using standard clinical tests and patient self-reporting. A finding that the patient's body weight, body fat, or percent body fat is reduced, or that the patient's serum lipid profile is improved following administration of the composition compared to test results prior to administration of the composition indicates that the patient is responding favorably to the treatment. Subsequent doses can be determined and administered as needed.

### Example 11—Treatment of Thrombocytopenia by Administration of an Extracellular ActRII Chimera

According to the methods disclosed herein, a physician of skill in the art can treat a subject, such as a human patient, having thrombocytopenia (e.g., thrombocytopenia associated with a myelodysplastic syndrome or myelofibrosis) so as to increase platelet levels (e.g., increase platelet count), increase platelet production, and/or increase megakaryocyte differentiation and/or maturation. To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat thrombocytopenia. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to increase platelet levels (e.g., increase platelet count), increase platelet production, and/or increase megakaryocyte differentiation and/or maturation.

Following administration of the composition to a patient, a practitioner of skill in the art can monitor the patient's improvement in response to the therapy by a variety of methods. For example, a physician can monitor the patient's platelet count using a blood test. A finding that the patient's platelet levels are increased (e.g., a finding of an increased platelet count) following administration of the composition compared to test results prior to administration of the composition indicates that the patient is responding favorably to the treatment. Subsequent doses can be determined and administered as needed.

### Example 12—Treatment of Neutropenia by Administration of an Extracellular ActRII Chimera

According to the methods disclosed herein, a physician of skill in the art can treat a subject, such as a human patient, having neutropenia (e.g., neutropenia associated with a myelodysplastic syndrome or myelofibrosis) so as to increase neutrophil levels (e.g., increase neutrophil count), increase neutrophil production, and/or increase the differentiation and/or maturation of progenitor cells (e.g., myeloid progenitors, myeloblasts, or myelocytes) into neutrophils. To treat the subject, a physician of skill in the art can administer to the subject a composition containing an extracellular ActRII chimera described herein (e.g., an extracellular ActRII chimera of Table 1 or Table 2, e.g., an extracellular ActRII chimera of any one of SEQ ID NOs: 96-126). The composition containing the extracellular ActRII chimera may be administered to the subject, for example, by parenteral injection (e.g., intravenous or subcutaneous injection) to treat neutropenia. The extracellular ActRII chimera is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered bimonthly, once a month, once every four weeks, once every two weeks, or at least once a week or more (e.g., 1, 2, 3, 4, 5, 6, or 7 times a week or more). The extracellular ActRII chimera is administered in an amount sufficient to increase neutrophil levels (e.g., increase neutrophil count), increase neutrophil production, and/or increase the differentiation and/or maturation of progenitor cells (e.g., myeloid progenitors, myeloblasts, or myelocytes) into neutrophils.

Following administration of the composition to a patient, a practitioner of skill in the art can monitor the patient's improvement in response to the therapy by a variety of methods. For example, a physician can monitor the patient's neutrophil count using a blood test. A finding that the patient's neutrophil levels are increased (e.g., a finding of an increased neutrophil count) following administration of the composition compared to test results prior to administration of the composition indicates that the patient is responding favorably to the treatment. Subsequent doses can be determined and administered as needed.

### Example 13—Treatment of End-Stage Renal Disease by Administration of an Extracellular ActRII Chimera

According to the methods disclosed herein, a physician of skill in the art can treat a subject, such as a human patient, having end-stage renal disease so as to increase EPO levels. To treat the subject, a physician of skill in the art can administer to the subject a composition containing a polypeptide including an extracellular ActRII chimera described herein (e.g., an extracellular ActRII chimera of Table 1 or Table 2, e.g., an extracellular ActRII chimera of any one of SEQ ID NOs: 96-126), such as a polypeptide containing an extracellular ActRII chimera described herein linked to an Fc domain. The composition containing the extracellular ActRII chimera may be administered to the subject, for example, by parenteral injection (e.g., intravenous or subcutaneous injection) to treat end-stage renal disease. The polypeptide containing an extracellular ActRII chimera described herein is administered in a therapeutically effective amount, such as from 0.01 to 500 mg/kg (e.g., 0.01, 0.1, 0.2, 0.3, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII chimera is administered once every sixteen weeks, quarterly, once every twelve weeks, bimonthly, once every eight weeks, once a month, once every four weeks, once every two weeks, or at least once a week or more (e.g., 1, 2, 3, 4, 5, 6, or 7 times a week or more). The extracellular ActRII chimera is administered in an amount sufficient to increase EPO levels, increase EPO receptor levels, and/or slow progression of the disease.

Following administration of the composition to a patient, a practitioner of skill in the art can monitor the patient's improvement in response to the therapy by a variety of methods. For example, a physician can monitor the patient's EPO levels using a blood test and can measure kidney function using blood tests, urine tests, and imaging tests. A finding that the patient's EPO levels are increased or that the disease is progressing more slowly following administration of the composition compared to test results prior to administration of the composition indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

Other embodiments are within the following claims.

1. A polypeptide comprising an extracellular activin receptor type II (ActRII) chimera, the chimera having a sequence of X₁β₁X₂β₂X₃β₃X₄β₄X₅β₅X₆β₆X₇β₇X₈, wherein:
X₁ is GAILGRSETQ (X₁ₐ) (SEQ ID NO: 1) or GRGEAETR (X_{1b}) (SEQ ID NO: 2);
β₁ is ECLFF (β₁ₐ) (SEQ ID NO: 3) or ECIYY (β_{1b}) (SEQ ID NO: 4);
X₂ is NANWEKDRTN (X₂ₐ) (SEQ ID NO: 5) or NANWELERTN (X₂) (SEQ ID NO: 6);
β₂ is QTGVEPC (β₂ₐ) (SEQ ID NO: 7) or QSGLERC (β_{2b}) (SEQ ID NO: 8);
X₃ is YGDKDKR (X₃ₐ) (SEQ ID NO: 9) or (X_{3b}) EGEQDKR (SEQ ID NO: 10);
β₃ is RHCFATWKNI (Bsa) (SEQ ID NO: 11) or a portion thereof that comprises HCFATWK (SEQ ID NO: 12) or LHCYASWRNS (β_{3b}) (SEQ ID NO: 13) or a portion thereof that comprises HCYASWR (SEQ ID NO: 14), wherein when β₃ is HCFATWK or HCYASWR, the chimera comprises contiguous amino acids from ActRIIA or ActRIIB connecting β₃ to X₃ and X₄;
X₄ is SG;
β₄ is SIEIVKQGCW (β₄ₐ) (SEQ ID NO: 15) or a portion thereof that comprises EIVKQGCW (SEQ ID NO: 16) or TIELVKKGCW (β_{4b}) (SEQ ID NO: 17) or a portion thereof that comprises ELVKKGCW (SEQ ID NO: 18), wherein when β₄ is EIVKQGCW or ELVKKGCW, the chimera comprises contiguous amino acids from ActRIIA or ActRIIB connecting β₄ to X₄;
X₅ is LDDINCYDRTDC (X₅ₐ) (SEQ ID NO: 19) or LDDENCYDRQEC (X₅%) (SEQ ID NO: 20);
β₅ is VEK (β₅ₐ) or a portion thereof that comprises VE or VAT (β_{5b}) or a portion thereof that comprises V, wherein when β₅ is VE or V, the chimera comprises contiguous amino acids from ActRIIA or ActRIIB connecting β5 to X₆;
X₆ is KDSPEV (X₆ₐ) (SEQ ID NO: 21) or EENPQV (X₆) (SEQ ID NO: 22);
β₆ is YFCCCE (SEQ ID NO: 23);
X₇ is GNMCNE (X₇ₐ) (SEQ ID NO: 24) or GNFCNE (X_{7b}) (SEQ ID NO: 25);
β₇ is KFSYF (δ₇ₐ) (SEQ ID NO: 26) or a portion thereof that comprises SYF or RFTHL (β_{7b}) (SEQ ID NO: 27) or a portion thereof that comprises T, wherein when β₇ is SYF or T, the chimera comprises contiguous amino acids from ActRIIA or ActRIIB connecting β₇ to X₇ and X₈; and
X₈ is PEMEVTQPTS (X₈ₐ) (SEQ ID NO: 28) or PEAGGPEVTYEPPPTAPT (X₈) (SEQ ID NO: 29), wherein:
(i) at least one of β₁ₐ, β₂ₐ, β₃ₐ, β₄ₐ, β₅ₐ, or β₇ₐ and at least one of β_{1b}, β_{2b}, β_{3b}, β_{4b}, β_{5b}, or β_{7b} is present in the chimera, and/or
(ii) at least one of X₁ₐ, X₂ₐ, X₃ₐ, X₅ₐ, X₆ₐ, X₇ₐ, or X₈ₐ and at least one of X_{1b}, X_{2b}, X_{3b}, X_{5b}, X_{6b}, X_{7b}, or X_{8b} is present in the chimera,
optionally wherein the chimera is truncated from the N-terminus by deletion of 1, 2, 3, 4, 5, 6, 7, 8, or 9 amino acids, wherein the chimera retains the two amino acids before the first cysteine.

2. The polypeptide of claim 1, wherein X₁ is GAILGRSETQ (X₁ₐ).

3. The polypeptide of claim 1, wherein X₁ is GRGEAETR (X_{1b}).

4. The polypeptide of any one of claims 1-3, wherein β₁ is ECLFF (β₁ₐ).

5. The polypeptide of any one of claims 1-3, wherein β₁ is ECIYY (β_{1b}).

6. The polypeptide of any one of claims 1-5, wherein X₂ is NANWEKDRTN (X₂ₐ).

7. The polypeptide of any one of claims 1-5, wherein X₂ is NANWELERTN (X₂).

8. The polypeptide of any one of claims 1-7, wherein β₂ is QTGVEPC (β₂ₐ).

9. The polypeptide of any one of claims 1-7, wherein β₂ is QSGLERC (β_{2b}).

10. The polypeptide of any one of claims 1-9, wherein X₃ is YGDKDKR (X₃ₐ).

11. The polypeptide of any one of claims 1-9, wherein X₃ is EGEQDKR (X_{3b}).

12. The polypeptide of any one of claims 1-11, wherein β₃ is RHCFATWKNI (β₃ₐ).

13. The polypeptide of any one of claims 1-11, wherein β₃ is LHCYASWRNS (β_{3b}).

14. The polypeptide of any one of claims 1-11, wherein β₃ is HCFATWK, wherein the chimera comprises contiguous amino acids from ActRIIA or ActRIIB connecting β₃ to X₃ and X₄.

15. The polypeptide of any one of claims 1-11, wherein β₃ is HCYASWR, wherein the chimera comprises contiguous amino acids from ActRIIA or ActRIIB connecting β₃ to X₃ and X₄.

16. The polypeptide of claim 14 or 15, wherein the contiguous amino acids connecting β₃ to X₃ are from ActRIIA.

17. The polypeptide of claim 14 or 15, wherein the contiguous amino acids connecting β₃ to X₃ are from ActRIIB.

18. The polypeptide of any one of claims 14-17, wherein the contiguous amino acids connecting β₃ to X₄ are from ActRIIA.

19. The polypeptide of any one of claims 14-17, wherein the contiguous amino acids connecting β₃ to X₄ are from ActRIIB.

20. The polypeptide of any one of claims 1-19, wherein β₄ is SIEIVKQGCW (β₄ₐ).

21. The polypeptide of any one of claims 1-19, wherein β₄ is TIELVKKGCW (β_{4b}).

22. The polypeptide of any one of claims 1-19, wherein β₄ is EIVKQGCW, wherein the chimera comprises contiguous amino acids from ActRIIA or ActRIIB connecting β₄ to X₄.

23. The polypeptide of any one of claims 1-19, wherein β₄ is ELVKKGCW, wherein the chimera comprises contiguous amino acids from ActRIIA or ActRIIB connecting β⁴ to X₄.

24. The polypeptide of claim 22 or 23, wherein the contiguous amino acids connecting β₄ to X₄ are from ActRIIA.

25. The polypeptide of claim 22 or 23, wherein the contiguous amino acids connecting β₄ to X₄ are from ActRIIB.

26. The polypeptide of any one of claims 1-25, wherein X₅ is LDDINCYDRTDC (X₅ₐ).

27. The polypeptide of any one of claims 1-25, wherein X₅ is LDDFNCYDRQEC (X_{5b}).

28. The polypeptide of any one of claims 1-27, wherein β₅ is VEK (β₅ₐ).

29. The polypeptide of any one of claims 1-27, wherein β₅ is VAT (β_{5b}).

30. The polypeptide of any one of claims 1-27, wherein β₅ is VE, wherein the chimera comprises contiguous amino acids from ActRIIA or ActRIIB connecting β₅ to X₆.

31. The polypeptide of any one of claims 1-27, wherein β₃ is V, wherein the chimera comprises contiguous amino acids from ActRIIA or ActRIIB connecting β₅ to X₆.

32. The polypeptide of claim 30 or 31, wherein the contiguous amino acids connecting β₅ to X₆ are from ActRIIA.

33. The polypeptide of claim 30 or 31, wherein the contiguous amino acids connecting β₅ to X₆ are from ActRIIB.

34. The polypeptide of any one of claims 1-33, wherein X₆ is KDSPEV (X₆ₐ).

35. The polypeptide of any one of claims 1-33, wherein X₆ is EENPQV (X_{6b}).

36. The polypeptide of any one of claims 1-35, wherein X₇ is GNMCNE (X₇ₐ).

37. The polypeptide of any one of claims 1-35, wherein X₇ is GNFCNE (X_{7b}).

38. The polypeptide of any one of claims 1-37, wherein β₇ is KFSYF (β₇ₐ).

39. The polypeptide of any one of claims 1-37, wherein β₇ is RFTHL (β_{7b}).

40. The polypeptide of any one of claims 1-37, wherein β₇ is SYF, wherein the chimera comprises contiguous amino acids from ActRIIA or ActRIIB connecting β₇ to X₇.

41. The polypeptide of any one of claims 1-37, wherein β₇ is T, wherein the chimera comprises contiguous amino acids from ActRIIA or ActRIIB connecting β₇ to X₇ and X₈.

42. The polypeptide of claim 40 or 41, wherein the contiguous amino acids connecting β₇ to X₇ are from ActRIIA.

43. The polypeptide of claim 40 or 41, wherein the contiguous amino acids connecting β₇ to X₇ are from ActRIIB.

44. The polypeptide of any one of claims 41-43, wherein the contiguous amino acids connecting β₇ to X₈ are from ActRIIA.

45. The polypeptide of any one of claims 41-43, wherein the contiguous amino acids connecting β₇ to X₈ are from ActRIIB.

46. The polypeptide of any one of claims 1-45, wherein X₈ is PEMEVTQPTS (X₈ₐ).

47. The polypeptide of any one of claims 1-45, wherein X₈ is PEAGGPEVTYEPPPTAPT (X_{8b}).

48. The polypeptide of claim 1, wherein the ActRII chimera has the sequence of any one of SEQ ID NOs: 96-126.

49. The polypeptide of any one of claims 1-48, wherein the chimera is truncated from the N-terminus by deletion of one amino acid.

50. The polypeptide of any one of claims 1-48, wherein the chimera is truncated from the N-terminus by deletion of two amino acids.

51. The polypeptide of any one of claims 1-48, wherein the chimera is truncated from the N-terminus by deletion of three amino acids.

52. The polypeptide of any one of claims 1-48, wherein the chimera is truncated from the N-terminus by deletion of four amino acids.

53. The polypeptide of any one of claims 1-48, wherein the chimera is truncated from the N-terminus by deletion of five amino acids.

54. The polypeptide of any one of claims 1-48, wherein the chimera is truncated from the N-terminus by deletion of six amino acids.

55. The polypeptide of any one of claims 1-48, wherein the chimera is truncated from the N-terminus by deletion of seven amino acids.

56. The polypeptide of any one of claims 1-48, wherein the chimera is truncated from the N-terminus by deletion of eight amino acids.

57. The polypeptide of any one of claims 1-48, wherein the chimera is truncated from the N-terminus by deletion of nine amino acids.

58. The polypeptide of any one of claims 1-57, further comprising a C-terminal extension of one or more amino acids.

59. The polypeptide of claim 58, wherein the C-terminal extension is NP.

60. The polypeptide of claim 58, wherein the C-terminal extension is NPVTPK (SEQ ID NO: 91).

61. The polypeptide of any one of claims 1-60, further comprising an Fc domain monomer fused to the C-terminus of the polypeptide by way of a linker.

62. The polypeptide of claim 61, wherein the Fc domain monomer comprises the sequence of SEQ ID NO: 34.

63. The polypeptide of claim 61 or 62, wherein the polypeptide forms a dimer.

64. The polypeptide of any one of claims 1-60, further comprising an Fc domain fused to the C-terminus of the polypeptide by way of a linker.

65. The polypeptide of claim 64, wherein the Fc domain comprises the sequence of SEQ ID NO: 87 or SEQ ID NO: 35.

66. The polypeptide of claim 64, wherein the Fc domain does not form a dimer.

67. The polypeptide of any one of claims 1-60, further comprising an albumin-binding peptide fused to the C-terminus of the polypeptide by way of a linker.

68. The polypeptide of claim 67, wherein the albumin-binding peptide comprises the sequence of SEQ ID NO: 88.

69. The polypeptide of any one of claims 1-60, further comprising a fibronectin domain fused to the C-terminus of the polypeptide by way of a linker.

70. The polypeptide of claim 69, wherein the fibronectin domain comprises the sequence of SEQ ID NO: 89.

71. The polypeptide of any one of claims 1-60, further comprising a human serum albumin fused to the C-terminus of the polypeptide by way of a linker.

72. The polypeptide of claim 71, wherein the human serum albumin comprises the sequence of SEQ ID NO: 90.

73. The polypeptide of any one of claims 61-72, wherein the linker is an amino acid spacer.

74. The polypeptide of claim 73, wherein the amino acid spacer is GGG, GGGA (SEQ ID NO: 36), GGGG (SEQ ID NO: 38), GGGAG (SEQ ID NO: 68), GGGAGG (SEQ ID NO: 69), GGGAGGG (SEQ ID NO: 70), GGS, GGGS (SEQ ID NO: 39), GGGGS (SEQ ID NO: 42), GGSG (SEQ ID NO: 45), or SGGG (SEQ ID NO: 47).

75. The polypeptide of any one of claims 1-74, wherein the polypeptide has a serum half-life of at least seven days.

76. The polypeptide of any one of claims 1-75, wherein the polypeptide binds to activin A, activin B, and/or myostatin and has reduced or weak binding to human BMP9.

77. The polypeptide of claim 75 or 76, wherein the polypeptide does not substantially bind to human BMP9.

78. The polypeptide of any one of claims 1-77, wherein the polypeptide binds to human activin A with a K_{D} of 800 pM or less.

79. The polypeptide of any one of claims 1-78, wherein the polypeptide binds to human activin B with a K_{D} of 800 pM or less.

80. The polypeptide of any one of claims 1-79, wherein the polypeptide binds to human GDF-11 with a K_{D} of 800 pM or less.

81. The polypeptide of any one of claims 1-80, wherein the polypeptide binds to human GDF-8 with a K_{D} of 800 pM or less.

82. A nucleic acid molecule encoding the polypeptide of any one of claims 1-81.

83. A vector comprising the nucleic acid molecule of claim 82.

84. A host cell that expresses the polypeptide of any one of claims 1-81, wherein the host cell comprises the nucleic acid molecule of claim 82 or the vector of claim 83, wherein the nucleic acid molecule or vector is expressed in the host cell.

85. A method of preparing the polypeptide of any one of claims 1-81, the method comprising:
a) providing a host cell comprising the nucleic acid molecule of claim 82 or the vector of claim 83, and
b) expressing the nucleic acid molecule or vector in the host cell under conditions that allow for the formation of the polypeptide

86. A pharmaceutical composition comprising the polypeptide of any one of claims 1-81, the nucleic acid molecule of claim 82, or the vector of claim 83, and one or more pharmaceutically acceptable carriers or excipients.

87. A method of increasing lean mass and/or muscle mass in a subject in need thereof, comprising administering to the subject a therapeutically effective amount of the polypeptide of any one of claims 1-81, the nucleic acid molecule of claim 82, the vector of claim 83, or the pharmaceutical composition of claim 86.

88. A method of treating a subject having or at risk of developing a disease or condition involving muscle weakness or atrophy, comprising administering to the subject a therapeutically effective amount of the polypeptide of any one of claims 1-81, the nucleic acid molecule of claim 82, the vector of claim 83, or the pharmaceutical composition of claim 86.

89. The method of claim 87 or 88, wherein the subject has or is at risk of developing a neuromuscular disease, sarcopenia, cachexia, disuse atrophy, treatment-related muscle loss or atrophy, hypotonia, muscle loss or atrophy associated with hypoxia, or muscle loss or atrophy associated with a burn injury.

90. A method of treating a subject having or at risk of developing a neuromuscular disease, comprising administering to the subject a therapeutically effective amount of the polypeptide of any one of claims 1-81, the nucleic acid molecule of claim 82, the vector of claim 83, or the pharmaceutical composition of claim 86.

91. A method of treating a subject having or at risk of developing a bone disease, comprising administering to the subject a therapeutically effective amount of the polypeptide of any one of claims 1-81, the nucleic acid molecule of claim 82, the vector of claim 83, or the pharmaceutical composition of claim 86.

92. The method of claim 91, wherein the bone disease is osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss.

93. A method of treating a subject having or at risk of developing fibrosis, comprising administering to the subject a therapeutically effective amount of the polypeptide of any one of claims 1-81, the nucleic acid molecule of claim 82, the vector of claim 83, or the pharmaceutical composition of claim 86.

94. The method of claim 93, wherein the fibrosis is chemotherapeutic drug-induced fibrosis, radiation-induced fibrosis, pulmonary fibrosis, hepatic fibrosis, renal fibrosis, corneal fibrosis, heart fibrosis, bone marrow fibrosis, myelofibrosis, mediastinal fibrosis, retroperitoneal fibrosis, osteoarticular fibrosis, arthrofibrosis, tissue fibrosis, a tumor stroma, a desmoplastic tumor, a surgical adhesion, a hypertrophic scar, or a keloid.

95. The method of claim 94, wherein the tissue fibrosis is fibrosis affecting a tissue selected from the group consisting of muscle tissue, skin epidermis, skin dermis, tendon, cartilage, pancreatic tissue, uterine tissue, neural tissue, testis, ovary, adrenal gland, artery, vein, bone marrow, colon, small intestine, large intestine, biliary tract, and gut.

96. The method of claim 93, wherein the fibrosis is fibrosis associated with a wound, a burn, hepatitis B or C infection, fatty liver disease, Schistosoma infection, kidney disease, chronic kidney disease, heart disease, macular degeneration, retinal or vitreal retinopathy, Crohn's disease, systemic or local scleroderma, atherosclerosis, or restenosis.

97. A method of treating a subject having or at risk of developing anemia, comprising administering to the subject a therapeutically effective amount of the polypeptide of any one of claims 1-81, the nucleic acid molecule of claim 82, the vector of claim 83, or the pharmaceutical composition of claim 86.

98. The method of claim 97, wherein the anemia is associated with cancer, cancer treatment, myelofibrosis treatment, chronic kidney disease, acute renal disease or failure, chronic renal disease or failure, a myelodysplastic syndrome, thalassemia, a nutritional deficit, adverse reaction to medication, ineffective hematopoiesis, an inflammatory or autoimmune disease, splenomegaly, porphyria, vasculitis, hemolysis, a bone marrow defect, bone marrow transplantation, myelofibrosis, diabetes, acute liver disease, chronic liver disease, acute bleeding, chronic bleeding, an infection, hemoglobinopathy, drug use, alcohol abuse, Churg-Strauss syndrome, Felty syndrome, Pearson syndrome, dyskeratosis congenita, graft versus host disease, hematopoietic stem cell transplantation, osteomyelofibrosis, pancytopenia, pure red-cell aplasia, purpura Schoenlein-Henoch, Shwachman Diamond syndrome, advanced age, contraindication to transfusion, surgery, trauma, a wound, an ulcer, urinary tract bleeding, digestive tract bleeding, frequent blood donation, or heavy menstrual bleeding.

99. The method of claim 97, wherein the anemia is aplastic anemia, iron deficiency anemia, vitamin deficiency anemia, anemia of chronic disease, anemia associated with a bone marrow disease, hemolytic anemia, sickle cell anemia, microcytic anemia, hypochromic anemia, congenital dyserythropoietic anemia, sideroblastic anemia, Diamond Blackfan anemia, Fanconi anemia, or refractory anemia with excess of blasts.

100. A method of treating a subject having or at risk of developing thrombocytopenia, comprising administering to the subject a therapeutically effective amount of the polypeptide of any one of claims 1-81, the nucleic acid molecule of claim 82, the vector of claim 83, or the pharmaceutical composition of claim 86.

101. The method of claim 100, wherein the thrombocytopenia is associated with a bone marrow defect, a myelodysplastic syndrome, bone marrow transplantation, myelofibrosis, myelofibrosis treatment, ineffective hematopoiesis, Gaucher disease, aplastic anemia, Fanconi anemia, Diamond Blackfan anemia, Shwachman Diamond syndrome, heavy alcohol consumption, cirrhosis of the liver, cancer, an autoimmune disease, a viral infection, a bacterial infection, an enlarged spleen, a vitamin deficiency, cancer treatment, thrombotic thrombocytopenia purpura, idiopathic thrombocytopenia purpura, disseminated intravascular coagulation, hemolytic uremic syndrome, paroxysmal nocturnal hemoglobinuria, a reduction of platelets caused by medication, acquired amegakaryocytic thrombocytopenia, Pearson syndrome, dyskeratosis congenita, a dilution of platelets caused by a blood transfusion, hematopoietic stem cell transplantation, or contraindication to transfusion.

102. The method of claim 100, wherein the thrombocytopenia is familial thrombocytopenia.

103. The method of claim 102, wherein the familial thrombocytopenia is May-Hegglin anomaly, Sebastian syndrome, Fechtner syndrome, Epstein's syndrome, Wiskott-Aldrich syndrome, congenital amegakaryocytic thrombocytopenia, platelet storage pool deficiency, Hermansky-Pudlak syndrome, Bernard-Soulier syndrome, Von Willebrand Disease Type 2B, ANKRD26-related thrombocytopenia, thrombocytopenia absent radius syndrome, familial platelet disorder with associated myeloid malignancy (FPD/AML), thrombocytopenia associated with a mutation in Filamin-A, or thrombocytopenia associated with a mutation in GATA-1.

104. The method of claim 100, wherein the thrombocytopenia is immune thrombocytopenia.

105. A method of treating a subject having or at risk of developing neutropenia, comprising administering to the subject a therapeutically effective amount of the polypeptide of any one of claims 1-81, the nucleic acid molecule of claim 82, the vector of claim 83, or the pharmaceutical composition of claim 86.

106. The method of claim 105, wherein the neutropenia is associated with a bone marrow defect, a myelodysplastic syndrome, bone marrow transplantation, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer, a vitamin deficiency, an enlarged spleen, an autoimmune disease, a viral infection, a bacterial infection, cancer treatment, a reduction in neutrophils caused by medication, inflammation, hematopoietic stem cell transplantation, or contraindication to transfusion.

107. The method of claim 105, wherein the neutropenia is familial neutropenia.

108. The method of claim 107, wherein the familial neutropenia is cyclic neutropenia, chronic benign neutropenia, or severe congenital neutropenia.

109. The method of claim 105, wherein the neutropenia is chronic idiopathic neutropenia.

110. A method of treating a subject having or at risk of developing PH, comprising administering to the subject a therapeutically effective amount of the polypeptide of any one of claims 1-81, the nucleic acid molecule of claim 82, the vector of claim 83, or the pharmaceutical composition of claim 86.

111. The method of claim 110, wherein the PH is pulmonary arterial hypertension (PAH), venous PH, hypoxic PH, thromboembolic PH, or miscellaneous PH.

112. A method of treating and/or preventing a metabolic disease in a subject, said method comprising administering to said subject a therapeutically effective amount of the polypeptide of any one of claims 1-81, the nucleic acid molecule of claim 82, the vector of claim 83, or the pharmaceutical composition of claim 86.

113. The method of claim 112, wherein the metabolic disease is age-related metabolic disease or treatment-related metabolic disease.

114. The method of claim 112 or 113, wherein the metabolic disease is obesity, Type 1 diabetes, or Type 2 diabetes.

115. A method of treating a subject having a disease or condition that can be treated with erythropoietin or an erythropoiesis-stimulating agent, comprising administering to the subject a therapeutically effective amount of the polypeptide of any one of claims 1-81, the nucleic acid molecule of claim 82, the vector of claim 83, or the pharmaceutical composition of claim 86.

116. A method of increasing erythropoietin levels and/or erythropoietin receptor levels in a subject in need thereof, comprising administering to the subject a therapeutically effective amount of the polypeptide of any one of claims 1-81, the nucleic acid molecule of claim 82, the vector of claim 83, or the pharmaceutical composition of claim 86.

117. The method of claim 115 or 116, wherein the subject has or is at risk of developing anemia due to dialysis or anemia of prematurity.

118. The method of claim 115 or 116, wherein the subject has or is at risk of developing of end-stage renal disease, renal insufficiency, polycythemia, hemochromatosis, a disease or condition associated with dysfunction of endothelial progenitor cells, a disease or condition having an autoimmune or inflammatory component, a neurological disorder or inflammatory brain disease, gastrointestinal dysmotility, a disease of the endocrine system, a disease of the reproductive system, aging, pregnancy, a menstrual disorder, ischemia or an ischemic disorder or condition, hypoxia or a hypoxic disorder or condition, an ulcer, a burn, a wound, ischemia-reperfusion injury, asthma, hypertension, a viral disease or infection, a systemic microbial infection, a gastrointestinal disease, arterial sclerosis, cancer, psychosis, a genetic disease, an inflammatory disease, graft-versus-host disease, cardiovascular disease, an allergy, or arthritis.

119. The method of claim 118, wherein:
(a) the ischemia is central nervous system ischemia, liver ischemia, renal ischemia, or cardiac ischemia;
(b) the ischemic disorder or condition is occlusive arterial disease, chronic venous insufficiency, circulatory shock, pulmonary embolism, myocardial infarction, ischemic stroke, acute respiratory failure, chronic heart failure, atherosclerosis, cardiac cirrhosis, macular degeneration, sleep apnea, Raynaud's disease, systemic sclerosis, nonbacterial thrombotic endocarditis, a transient ischemic attack, or ischemia resulting from general anesthesia;
(c) the hypoxic disorder or condition is a pulmonary disorder, severe pneumonia, pulmonary edema, hyaline membrane disease, liver disease, renal disease, cancer, or altitude sickness;
(d) the disease or condition associated with dysfunction of endothelial progenitor cells is heart failure, angina pectoris, endotheliosis, reticuloendotheliosis, age-related cardiovascular disorder, coronary heart disease, atherosclerosis, myocardial ischemia, hypercholesterolemia, an ischemic disorder of the extremities, Raynaud's disease, preeclampsia, pregnancy induced hypertension, an endothelium-mediated chronic inflammatory disorder, wound healing, chronic renal failure, or acute renal failure; and/or
(e) the autoimmune or inflammatory disease or condition is acute cerebrovascular injury, acute brain injury, acute cardiovascular injury, arthritis, an autoimmune disease, a stroke, a neurological injury, or immune-mediated inflammation.

120. A method of preparing a tissue or organ for transplantation, comprising contacting the tissue or organ with a therapeutically effective amount of the polypeptide of any one of claims 1-81, the nucleic acid molecule of claim 82, the vector of claim 83, or the pharmaceutical composition of claim 86.
